(19) United States
(12) Patent Application Publication
(10) Pub. No.: US 2020/0392071 A1
(43) Pub. Date: Dec. 17, 2020

(54) Gene cluster for the biosynthetic production of tetracycline compounds in a heterologous host

(71) Applicants: HELMHOLTZ-ZENTRUM FÜR INFEKTIONSFORSCHUNG GMBH, Braunschweig (DE); ACIES BIO D.O.O., Ljubljana (SI)

(72) Inventors: Rolf MÜLLER, Blieskastel (DE); Tadeja LUKEZIC, Ljubljana (SI); Maja REMSKAR, Saarbrücken (DE); Nestor ZABURANNYI, Saarbrücken (DE); Chantal BADER, St. Ingbert (DE); Asfandyar SIKANDAR, Saarbrücken (DE); Jesko KÖHNKE, Mandelbachtal (DE)

(21) Appl. No.: 16/954,461

(22) PCT Filed: Dec. 21, 2018
(86) PCT No.: PCT/EP2018/086740

§ 371 Date: Jun. 16, 2020

(30) Foreign Application Priority Data
Dec. 22, 2017 (EP) 17210536.3

(51) Int. Cl. C07C 237/26; C12N 15/63; C07K 14/36

(52) U.S. Cl. CPC C07C 237/26; C12N 2800/101; C07K 14/36; C12N 15/63

(57) ABSTRACT

The present invention relates to the application of biosynthetic engineering for the heterologous expression of a gene cluster for the biosynthesis of tetracycline compounds, notably chelocardin and its analogues. More particularly, the present invention pertains to a gene cluster encoding polypeptides involved in tetracycline biosynthesis, which gene cluster is suitable for heterologous expression of the biosynthetic pathway in a host cell. The present invention further pertains to DNA construct s comprising the gene cluster, to recombinant heterologous host cell s comprising the gene cluster or the DNA construct, to processes for the biosynthetic production of a tetracycline compound employing such recombinant host cells, and to tetracycline compounds thereby produced. The present invention also pertains to fusion proteins which are useful in the production of tetracycline compounds.

### FIELD OF THE INVENTION

The present invention relates to the application of biosynthetic engineering for the heterologous expression of a gene cluster for the biosynthesis of tetracycline compounds, notably chelocardin and its analogues. More particularly, the present invention pertains to a gene cluster encoding polypeptides involved in tetracycline biosynthesis, which gene cluster is suitable for heterologous expression of the biosynthetic pathway in a host cell. The present invention further pertains to DNA constructs comprising the gene cluster, to recombinant heterologous host cells comprising the gene cluster or the DNA construct, to processes for the biosynthetic production of a tetracycline compound employing such recombinant host cells, and to tetracycline compounds thereby produced. The present invention also pertains to fusion proteins which are useful in the production of tetracycline compounds.

### BACKGROUND OF THE INVENTION

Chelocardin (CHD; also known as M319, cetocycline or cetotetrine) is an atypical tetracycline with broad spectrum of antibiotic activity, produced by the actinomycete Amycolatopsis sulphurea. Possession of a well-known tetracycline scaffold is only one the structural characteristics of chelocardin. Importantly, chelocardin is also active against tetracycline-resistant pathogens (Proctor et al. 1978). It showed promising in a small phase II clinical study on patients with urinary tract infections caused by Gram-negative pathogens in 1977 (Molnar et al. 1977). Chelocardin structure differs in quite a number of details from the one of typical tetracyclines, reflecting also in a different mode of action (Rasmussen et al. 1991; Stepanek et al. 2016).

The use of genes from a chelocardin biosynthetic gene cluster would generally enable the production of the potent broad-spectrum antibiotic chelocardin and its analogues. The chelocardin biosynthetic gene cluster from Amycolatopsis sulphurea and its use has been described in EP2154249 (Petkovic et al.) and Lukezic et al. (Lukezic et al. 2013). However, while chelocardin and its analogues, especially its amidated analogue 2-carboxamido-2-deacetyl-chelocardin (CDCHD), could be obtained using the wild-type producer Amycolatopsis sulphurea or modified variants thereof, heterologous expression of the described gene cluster has turned out to be difficult as it did not result in the production of chelocardin in the heterologous host.

Accordingly, it is an object of the present invention to provide means which enable the production of chelocardin or analogues thereof in a heterologous host.

### SUMMARY OF THE INVENTION

The present invention is based on the surprising finding that the chelocardin biosynthetic gene cluster isolated from the wild-type chelocardin producer Amycolatopsis sulphurea comprises a further gene (herein referred to as chdY) encoding a second ring cyclase which seems to be essential for the formation of the basic tetracyclic scaffold. Even more surprising, the cyclase encoded by chdY gene and the FAD-dependent oxygenase encoded by the chdOII gene are expressed in the form of a fusion protein.

Moreover, the present inventors have identified two so far undiscovered regulatory genes within the chelocardin biosynthetic gene cluster (herein referred to as chdB and chdC, respectively) which encode transcriptional activators belonging to the SARP and LuxR family, respectively. While these regulatory genes are not directly involved in the synthesis of chelocardin, they are expected to have a positive effect on the production chelocardin in a heterologous host as seen for homologous family members in oxytetracycline production.

By providing the genetic information on the gene chdY as well as that of genes chdB and chdC, it is now possible to produce chelocardin and its analogues in a heterologous host, which in turn allows a higher production of this atypical tetracycline compared to the chelocardin natural producer, Amycolatopsis sulphurea.

The present invention therefore provides in a first aspect a gene cluster encoding polypeptides involved in the biosynthesis of a tetracycline.

The present invention provides in a further aspect a DNA construct comprising the gene cluster of the present invention.

The present invention provides in a further aspect a recombinant host cell comprising the gene cluster or the DNA construct according to the present invention.

The present invention provides in a further aspect a process for the biosynthetic production of a tetracycline, said method comprises the steps of a) cultivating a recombinant host cell according to the present invention in the presence of a suitable substrate under conditions conducive to the production of said tetracycline and, optionally, b) recovering the tetracycline from the cultivation medium.

The present invention provides in a further aspect fusion proteins of polypeptides involved in the biosynthesis of a tetracycline, and nucleic acid molecules encoding same.

The present invention can be further summarized by the following items:

1. A (isolated) gene cluster encoding polypeptides involved in the biosynthesis of a tetracycline, wherein said gene cluster includes all of the nucleotide sequences (1) to (19):

(1) a nucleotide sequence encoding a polypeptide which comprises an amino acid sequence having at least 80%, such as at least 85%, sequence identity with the polypeptide of SEQ ID NO: 1 and which has the same functional property as the polypeptide of SEQ ID NO: 1 [ChdP];

(2) a nucleotide sequence encoding a polypeptide which comprises an amino acid sequence having at least 80%, such as at least 85%, sequence identity with the polypeptide of SEQ ID NO: 2 and which has the same functional property as the polypeptide of SEQ ID NO: 2 [ChdK];

(3) a nucleotide sequence encoding a polypeptide which comprises an amino acid sequence having at least 80%, such as at least 85%, sequence identity with the polypeptide of SEQ ID NO: 3 and which has the same functional property as the polypeptide of SEQ ID NO: 3 [ChdS];

(4) a nucleotide sequence encoding a polypeptide which comprises an amino acid sequence having at least 80%, such as at least 85%, sequence identity with the polypeptide of SEQ ID NO: 4 and which has the same functional property as the polypeptide of SEQ ID NO: 4 [ChdQI];

(5) a nucleotide sequence encoding a polypeptide which comprises an amino acid sequence having at least 80%, such as at least 85%, sequence identity with the polypeptide of SEQ ID NO: 5 and which has the same functional property as the polypeptide of SEQ ID NO: 5 [ChdQII];

(6) a nucleotide sequence encoding a polypeptide which comprises an amino acid sequence having at least 80%, such as at least 85%, sequence identity with the polypeptide of SEQ ID NO: 6 and which has the same functional property as the polypeptide of SEQ ID NO: 6 [ChdX];

(7) a nucleotide sequence encoding a polypeptide which comprises an amino acid sequence having at least 80%, such as at least 85%, sequence identity with the polypeptide of SEQ ID NO: 7 and which has the same functional property as the polypeptide of SEQ ID NO: 7 [ChdL];

(8) a nucleotide sequence encoding a polypeptide which comprises an amino acid sequence having at least 80%, such as at least 85%, sequence identity with the polypeptide of SEQ ID NO: 8 and which has the same functional property as the polypeptide of SEQ ID NO: 8 [ChdT];

(9) a nucleotide sequence encoding a polypeptide which comprises an amino acid sequence having at least 80%, such as at least 85%, sequence identity with the polypeptide of SEQ ID NO: 9 and which has the same functional property as the polypeptide of SEQ ID NO: 9 [ChdMI];

(10) a nucleotide sequence encoding a polypeptide which comprises an amino acid sequence having at least 80%, such as at least 85%, sequence identity with the polypeptide of SEQ ID NO: 10 and which has the same functional property as the polypeptide of SEQ ID NO: 10 [ChdMII];

(11) a nucleotide sequence encoding a polypeptide which comprises an amino acid sequence having at least 80%, such as at least 85%, sequence identity with the polypeptide of SEQ ID NO: 11 and which has the same functional property as the polypeptide of SEQ ID NO: 11 [ChdN];

(12) a nucleotide sequence encoding a polypeptide which comprises an amino acid sequence having at least 80%, such as at least 85%, sequence identity with the polypeptide of SEQ ID NO: 12 and which has the same functional property as the polypeptide of SEQ ID NO: 12 [ChdGIV];

(13) a nucleotide sequence encoding a polypeptide which comprises an amino acid sequence having at least 80%, such as at least 85%, sequence identity with the polypeptide of SEQ ID NO: 13 and which has the same functional property as the polypeptide of SEQ ID NO: 13 [ChdTn];

(14) a nucleotide sequence encoding a polypeptide which comprises an amino acid sequence having at least 80%, such as at least 85%, sequence identity with the polypeptide of SEQ ID NO: 14 and which has the same functional property as the polypeptide of SEQ ID NO: 14 [ChdR];

(15) a nucleotide sequence encoding a polypeptide which comprises an amino acid sequence having at least 80%, such as at least 85%, sequence identity with the polypeptide of SEQ ID NO: 15 and which has the same functional property as the polypeptide of SEQ ID NO: 15 [ChdA];

(16) a nucleotide sequence encoding a polypeptide which comprises an amino acid sequence having at least 80%, such as at least 85%, sequence identity with the polypeptide of SEQ ID NO: 16 and which has the same functional property as the polypeptide of SEQ ID NO: 16 [ChdOI];

(17) a nucleotide sequence encoding a polypeptide which comprises an amino acid sequence having at least 80%, such as at least 85%, sequence identity with the polypeptide of SEQ ID NO: 17 and which has the same functional property as the polypeptide of SEQ ID NO: 17 [ChdOIII];

(18) a nucleotide sequence encoding a polypeptide which comprises an amino acid sequence having at least 80%, such as at least 85%, sequence identity with the polypeptide of SEQ ID NO: 18 and which has the same functional property as the polypeptide of SEQ ID NO: 18 [ChdOII]; and

(19) a nucleotide sequence encoding a polypeptide which comprises an amino acid sequence having at least 80%, such as at least 85%, sequence identity with the polypeptide of SEQ ID NO: 19 and which has the same functional property as the polypeptide of SEQ ID NO: 19 [ChdY].

2. The gene cluster according to item 1, wherein said gene cluster further comprises at least one of the nucleotide sequences (20) and (21):

(20) a nucleotide sequence encoding a polypeptide which comprises an amino acid sequence having at least 80%, such as at least 85%, sequence identity with the polypeptide of SEQ ID NO: 20 and which has the same functional property as the polypeptide of SEQ ID NO: 20 [SARP/ChdB]; and

(21) a nucleotide sequence encoding a polypeptide which comprises an amino acid sequence having at least 80%, such as at least 85%, sequence identity with the polypeptide of SEQ ID NO: 21 and which has the same functional property as the polypeptide of SEQ ID NO: 21 [LuxR/ChdC].

3. The gene cluster according to item 2, wherein said gene cluster further comprises both nucleotide sequences (20) and (21).

4. The gene cluster according to any one of items 1 to 3, wherein the nucleotide sequences (18) and (19) are linked to form a fusion protein of the respective polypeptides encoded by them.

5. The gene cluster according to item 4, wherein the fusion protein comprises an amino acid sequence having at least 80%, such as at least 85%, sequence identity with the polypeptide of SEQ ID NO: 22 and has the same functional properties as the polypeptides of SEQ ID NO: 18 and SEQ ID NO: 19 [ChdOII+ChdY].

6. The gene cluster according to any one of items 1 to 5, wherein the nucleotide sequences (7) and (17) are linked to form a fusion protein of the respective polypeptides encoded by them.

7. The gene cluster according to item 6, wherein the fusion protein comprises an amino acid sequence having at least 80%, such as at least 85%, sequence identity with the polypeptide of SEQ ID NO: 23 and has the same functional properties as the polypeptides of SEQ ID NO: 7 and SEQ ID NO: 17 [ChdL+ChdOIII].

8. A DNA construct comprising the gene cluster according to any one of items 1 to 7.

9. The DNA construct according to item 8, further comprises at least one nucleotide sequence selected from the nucleotide sequences (24) and (25):

(24) a nucleotide sequence encoding a polypeptide which comprises an amino acid sequence having at least 80%, such as at least 85%, sequence identity with the polypeptide of SEQ ID NO: 24 and has the same functional property as the polypeptides of SEQ ID NO: 24 [OxyD]; and

(25) a nucleotide sequence encoding a polypeptide which comprises an amino acid sequence having at least 80%, such as at least 85%, sequence identity with the polypeptide of SEQ ID NO: 25 and has the same functional property as the polypeptides of SEQ ID NO: 25 [OxyP].

10. The DNA construct according to item 8 or 9, wherein said DNA construct further comprises a nucleotide sequence encoding a polypeptide which comprises an amino acid sequence having at least 80%, such as at least 85%, sequence identity with the polypeptide of SEQ ID NO: 24 and which has the same functional property as the polypeptide of SEQ ID NO: 24 [OxyD].

11. The DNA construct according to any one of items 8 to 10, wherein said DNA construct further comprises a nucleotide sequence encoding a polypeptide which comprises an amino acid sequence having at least 80%, such as at least 85%, sequence identity with the polypeptide of SEQ ID NO: 25 and which has the same functional property as the polypeptide of SEQ ID NO: 25 [OxyP].

12. The DNA construct according to any one of items 8 to 11, wherein said DNA construct further comprises an additional nucleotide sequence encoding a polypeptide which comprises an amino acid sequence having at least 80%, such as at least 85%, sequence identity with the polypeptide of SEQ ID NO: 14 and which has the same functional property as the polypeptide of SEQ ID NO: 14 [ChdR].

13. The DNA construct according to any one of items 8 to 12, which is an expression cassette.

14. The DNA construct according to any one of items 8 to 13, which is a vector.

15. The DNA construct according to item 14, wherein the vector is a plasmid.

16. The DNA construct according to item 15, wherein the plasmid is a cosmid.

17. A recombinant host cell comprising the gene cluster according to any one of items 1 to 7 or the DNA construct according to any one of claims 8 to 16, wherein the gene cluster or DNA construct is heterologous to said host cell.

18. The recombinant host cell according to item 17, which heterologously expresses the polypeptides encoded by the gene cluster.

19. The recombinant host cell according to item 17 or 18, further comprising at least one nucleotide sequence selected from the nucleotide sequences (24) and (25):

(24) a nucleotide sequence encoding a polypeptide which comprises an amino acid sequence having at least 80%, such as at least 85%, sequence identity with the polypeptide of SEQ ID NO: 24 and has the same functional property as the polypeptides of SEQ ID NO: 24 [OxyD]; and

(25) a nucleotide sequence encoding a polypeptide which comprises an amino acid sequence having at least 80%, such as at least 85%, sequence identity with the polypeptide of SEQ ID NO: 25 and has the same functional property as the polypeptides of SEQ ID NO: 25 [OxyP].

20. The recombinant host cell according to item 19, which heterologously expresses at least one polypeptide which comprises an amino acid sequence having at least 80%, such as at least 85%, sequence identity with the polypeptide of SEQ ID NO: 24 or 25 and which has the same functional property as the polypeptide of SEQ ID NO: 24 or 25, respectively.

21. The recombinant host cell according to any one of items 17 to 20, which heterologously expresses a polypeptide which comprises an amino acid sequence having at least 80%, such as at least 85%, sequence identity with the polypeptide of SEQ ID NO: 24 and which has the same functional property as the polypeptide of SEQ ID NO: 24 [OxyD].

22. The recombinant host cell according to any one of items 17 to 21, which heterologously expresses a polypeptide which comprises an amino acid sequence having at least 80%, such as at least 85%, sequence identity with the polypeptide of SEQ ID NO: 25 and which has the same functional property as the polypeptide of SEQ ID NO: 25 [OxyP].

23. The recombinant host cell according to any one of items 17 to 22, wherein the gene cluster according to any one of items 1 to 7 or the DNA construct according to any one of items 8 to 19 is extrachromosomal.

24. The recombinant host cell according to any one of items 17 to 22, wherein the gene cluster according to any one of items 1 to 7 or the DNA construct according to any one of items 8 to 19 is integrated into one or more chromosomes of said host cell.

25. The recombinant host cell according to any one of items 17 to 24, which is a bacterium.

26. The recombinant host cell according to any one of items 17 to 25 which is a bacterium of the order Actinomycetales.

27. The recombinant host cell according to any one of items 17 to 26, which is a bacterium belonging to a genus selected from the group consisting of Streptomyces, Amycolatopsis and Nocardia.

28. The recombinant host cell according to any one of items 17 to 27, which is a bacterium selected from the group consisting of Streptomyces lividans, Streptomyces coelicolor, Streptomyces albus, Streptomyces rimosus, Amycolatopsis mediterranei, Amycolatopsis orientalis and Nocardia spp.

29. The recombinant host cell according to any one of items 17 to 28, which is Streptomyces albus.

30. A process for the biosynthetic production of a tetracycline, said process comprises the steps of a) cultivating a recombinant host cell according to any one of items 17 to 29 in the presence of a suitable substrate under conditions conducive to the production of said tetracycline and, optionally, b) recovering the tetracycline from the medium employed in cultivation.

31. The process according to item 30, wherein the tetracycline is chelocardin or an analogue thereof.

32. The process according to item 31, wherein the chelocardin is a compound having structure I

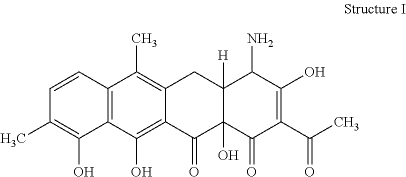 [SMILES: CC(=O)C1=C(O)C(N)C2Cc3c(c(O)c4c(O)c(C)ccc4c3C)C(=O)C2(O)C1=O]

optionally as a stereoisomer, including enantiomers and diastereomers, or in form of a mixture of at least two stereoisomers, including enantiomers and/or diastereomers, in any mixing ratio, or a corresponding salt thereof, or a corresponding solvate thereof.

33. The process according to item 32, wherein the chelocardin is in the form of a stereoisomer.

34. The process according to item 32 or 33, wherein the chelocardin has structure II

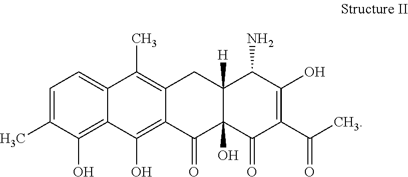 [SMILES: CC(=O)C1=C(O)[C@@H](N)[C@H]2Cc3c(c(O)c4c(O)c(C)ccc4c3C)C(=O)[C@@]2(O)C1=O]

35. The process according to item 31, wherein the chelocardin analogue is a compound having structure III

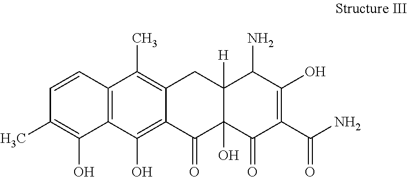 [SMILES: Cc1ccc2c(C)c3c(c(O)c2c1O)C(=O)C1(O)C(=O)C(C(N)=O)=C(O)C(N)C1C3]

optionally as a stereoisomer, including enantiomers and diastereomers, or in form of a mixture of at least two stereoisomers, including enantiomers and/or diastereomers, in any mixing ratio, or a corresponding salt thereof, or a corresponding solvate thereof.

36. The process according to item 35, wherein the chelocardin analogue is in the form of a stereoisomer.

37. The process according to item 35 or 36, wherein the chelocardin analogue has structure IV

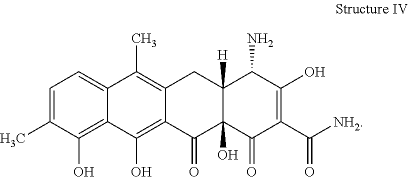 [SMILES: Cc1ccc2c(C)c3c(c(O)c2c1O)C(=O)[C@@]1(O)C(=O)C(C(N)=O)=C(O)[C@@H](N)[C@H]1C3]

38. A compound of structure I

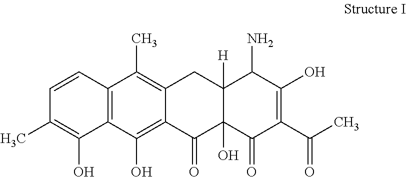 [SMILES: CC(=O)C1=C(O)C(N)C2Cc3c(c(O)c4c(O)c(C)ccc4c3C)C(=O)C2(O)C1=O]

optionally as a stereoisomer, including enantiomers and diastereomers, or in form of a mixture of at least two stereoisomers, including enantiomers and/or diastereomers, in any mixing ratio, or a corresponding salt thereof, or a corresponding solvate thereof.

39. The compound according to item 38, which is in the form of a stereoisomer.

40. The compound according to item 38 or 39 having structure II

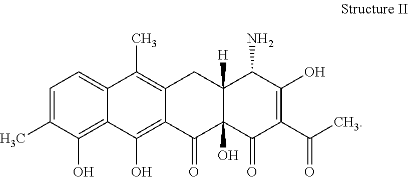 [SMILES: CC(=O)C1=C(O)[C@@H](N)[C@H]2Cc3c(c(O)c4c(O)c(C)ccc4c3C)C(=O)[C@@]2(O)C1=O]

41. A compound of structure III

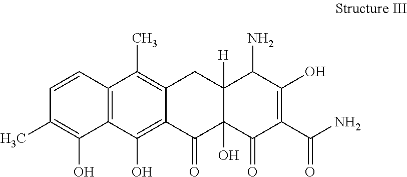 [SMILES: Cc1ccc2c(C)c3c(c(O)c2c1O)C(=O)C1(O)C(=O)C(C(N)=O)=C(O)C(N)C1C3]

optionally as a stereoisomer, including enantiomers and diastereomers, or in form of a mixture of at least two stereoisomers, including enantiomers and/or diastereomers, in any mixing ratio, or a corresponding salt thereof, or a corresponding solvate thereof.

42. The compound according to item 41, which is in the form of a stereoisomer.

43. The compound according to item 41 or 42 having the structure IV

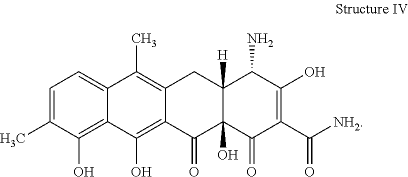 [SMILES: Cc1ccc2c(C)c3c(c(O)c2c1O)C(=O)[C@@]1(O)C(=O)C(C(N)=O)=C(O)[C@@H](N)[C@H]1C3]

44. The compound according to any one of items 38 to 43 for use as a medicament, such as in the treatment of a bacterial infection.

45. A (isolated) fusion protein comprises an amino acid sequence having at least 80%, such as 85%, sequence identity with the polypeptide of SEQ ID NO: 22.

46. The fusion protein according to item 45, which has FAD-dependent oxygenase and cyclase activities.

47. A (isolated) fusion protein comprises an amino acid sequence having at least 80%, such as 85%, sequence identity with the polypeptide of SEQ ID NO: 23

48. The fusion protein according to item 47, which has acyl-CoA ligase and oxygenase activities.

49. An nucleic acid molecule comprising a nucleotide sequence encoding the fusion protein according to any one of items 45 to 48.

### BRIEF DESCRIPTION OF THE DRAWINGS

FIG. 1: Chelocardin biosynthetic gene cluster, and genes involved in chelocardin production. A) Chelocardin biosynthetic gene cluster as found in A. sulphurea; B) Alternative gene cluster with chdL, chdOIII, chdY and chdOII present as separate genes.

FIG. 2: Proposed chelocardin biosynthesis pathway, according to the invention

FIG. 3: LC-MS analysis of culture extracts of S. albus with integrated cosmids carrying CHD biosynthetic gene cluster (pOJ436-CHD12) or CHD biosynthetic gene cluster with additional copy of chdR (pOJ436-PermE*-chdR-CHD12) in comparison with culture extracts of S. albus with integrated empty cosmids pOJ436 or pOJ436-PermE*-chdR. UV chromatograms at detection wavelength of 280 nm and EICs for m/z 412 (±0.5), which corresponds to CHD, are shown (chromatograms adapted from DataAnalysis (available from Bruker Daltonics)).

FIG. 4: LC-MS analysis of culture extracts of S. albus with integrated cosmid carrying CHD biosynthetic gene cluster together with oxyD and oxyP genes and additional copy of chdR (pOJ436-PermE*-oxyDPchdR-CHD12) in comparison with culture extracts of S. albus with integrated empty cosmid pOJ436-PermE*-oxyDPchdR. UV chromatograms at detection wavelength of 280 nm and EICs for m/z 412 (±0.5) and 413 (±0.5), which correspond to CHD and CDCHD, respectively, are shown (chromatograms adapted from DataAnalysis (available from Bruker Daltonics))

FIG. 5: LC-MS analysis of culture extracts of A. sulphurea wild-type (A) and A. sulphurea ChdY-G176S mutant (B). UV chromatograms at detection wavelength of 280 nm and EICs for m/z 412 (±0.5) and 369 (±0.5), corresponding to CHD and»compound 369«, respectively, are shown (chromatograms adapted from DataAnalysis (available from Bruker Daltonics)).

FIG. 6: CHD production in ChdY-G176S mutant before (ChdY-G176S+pAB03) and after complementation experiments with genes for either OxyN (ChdY-G176S+pAB03oxyN), ChdY (ChdY-G176S+pAB03chdY) or ChdOII-ChdY (ChdY-G176S+pAB03chdOII-chdY), compared to A. sulphurea WT with integrated empty plasmid pAB03 (WT+pAB03)

### DETAILED DESCRIPTION OF THE INVENTION

Unless specifically defined herein, all technical and scientific terms used have the same meaning as commonly understood by a skilled artisan in the fields of biochemistry, genetics, molecular biology and microbiology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, and recombinant DNA technology, which are within the skill of the art. Such techniques are explained fully in the literature. See, for example, Current Protocols in Molecular Biology (Ausubel 1987); Molecular Cloning: A Laboratory Manual, Third Edition, (Sambrook, Russell 2001); Transcription And Translation (Harnes, Higgins 1984); and the series, Methods In ENZYMOLOGY (Abelson, Simon 1998), specifically, Vols. 154 and 155 (Wu et al. eds.) and Vol. 185, “Gene Expression Technology” (D. Goeddel, ed.).

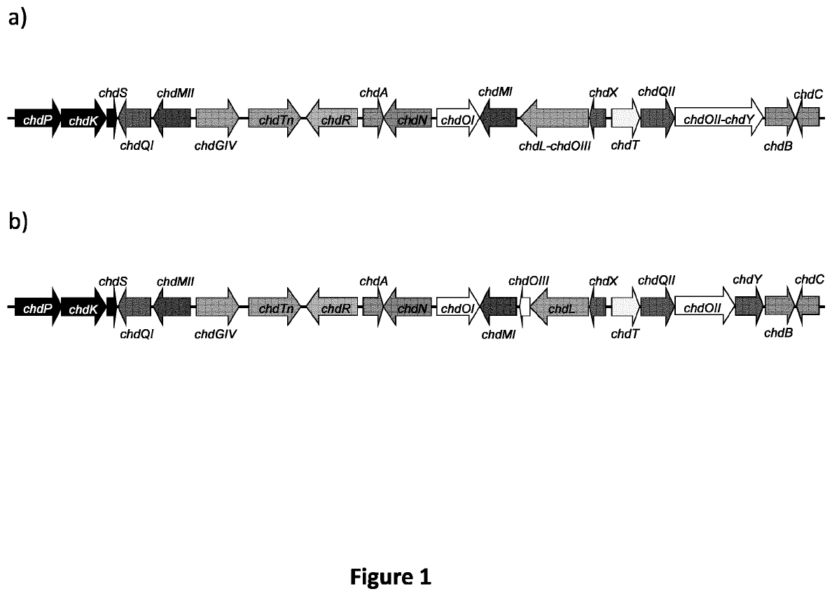

As mentioned above, the present invention provides a gene cluster encoding polypeptides involved in the biosynthesis of a tetracycline, notably chelocardin or an analogue thereof. A representative overview of the gene cluster gene cluster is presented in FIG. 1.

Particularly, the present invention provides a (isolated) gene cluster encoding polypeptides involved in the biosynthesis of a tetracycline, wherein said gene cluster includes all of the nucleotide sequences (1) to (19):

(1) a nucleotide sequence encoding a polypeptide which comprises an amino acid sequence having at least 80%, such as at least 85%, sequence identity with the polypeptide of SEQ ID NO: 1 [ChdP];

(2) a nucleotide sequence encoding a polypeptide which comprises an amino acid sequence having at least 80%, such as at least 85%, sequence identity with the polypeptide of SEQ ID NO: 2 [ChdK];

(3) a nucleotide sequence encoding a polypeptide which comprises an amino acid sequence having at least 80%, such as at least 85%, sequence identity with the polypeptide of SEQ ID NO: 3 [ChdS];

(4) a nucleotide sequence encoding a polypeptide which comprises an amino acid sequence having at least 80%, such as at least 85%, sequence identity with the polypeptide of SEQ ID NO: 4 [ChdQI];

(5) a nucleotide sequence encoding a polypeptide which comprises an amino acid sequence having at least 80%, such as at least 85%, sequence identity with the polypeptide of SEQ ID NO: 5 [ChdQII];

(6) a nucleotide sequence encoding a polypeptide which comprises an amino acid sequence having at least 80%, such as at least 85%, sequence identity with the polypeptide of SEQ ID NO: 6 [ChdX];

(7) a nucleotide sequence encoding a polypeptide which comprises an amino acid sequence having at least 80%, such as at least 85%, sequence identity with the polypeptide of SEQ ID NO: 7 [ChdL];

(8) a nucleotide sequence encoding a polypeptide which comprises an amino acid sequence having at least 80%, such as at least 85%, sequence identity with the polypeptide of SEQ ID NO: 8 [ChdT];

(9) a nucleotide sequence encoding a polypeptide which comprises an amino acid sequence having at least 80%, such as at least 85%, sequence identity with the polypeptide of SEQ ID NO: 9 [ChdMI];

(10) a nucleotide sequence encoding a polypeptide which comprises an amino acid sequence having at least 80%, such as at least 85%, sequence identity with the polypeptide of SEQ ID NO: 10 [ChdMII];

(11) a nucleotide sequence encoding a polypeptide which comprises an amino acid sequence having at least 80%, such as at least 85%, sequence identity with the polypeptide of SEQ ID NO: 11 [ChdN];

(12) a nucleotide sequence encoding a polypeptide which comprises an amino acid sequence having at least 80%, such as at least 85%, sequence identity with the polypeptide of SEQ ID NO: 12 [ChdGIV];

(13) a nucleotide sequence encoding a polypeptide which comprises an amino acid sequence having at least 80%, such as at least 85%, sequence identity with the polypeptide of SEQ ID NO: 13 [ChdTn];

(14) a nucleotide sequence encoding a polypeptide which comprises an amino acid sequence having at least 80%, such as at least 85%, sequence identity with the polypeptide of SEQ ID NO: 14 [ChdR];

(15) a nucleotide sequence encoding a polypeptide which comprises an amino acid sequence having at least 80%, such as at least 85%, sequence identity with the polypeptide of SEQ ID NO: 15 [ChdA];

(16) a nucleotide sequence encoding a polypeptide which comprises an amino acid sequence having at least 80%, such as at least 85%, sequence identity with the polypeptide of SEQ ID NO: 16 [ChdOI];

(17) a nucleotide sequence encoding a polypeptide which comprises an amino acid sequence having at least 80%, such as at least 85%, sequence identity with the polypeptide of SEQ ID NO: 17 [ChdOIII];

(18) a nucleotide sequence encoding a polypeptide which comprises an amino acid sequence having at least 80%, such as at least 85%, sequence identity with the polypeptide of SEQ ID NO: 18 [ChdOII]; and

(19) a nucleotide sequence encoding a polypeptide which comprises an amino acid sequence having at least 80%, such as at least 85%, sequence identity with the polypeptide of SEQ ID NO: 19 [ChdY].

According to certain embodiments, the polypeptide encoded by the nucleotide sequence (1) comprises an amino acid sequence having at least 85% sequence identity with the polypeptide of SEQ ID NO: 1. According to certain embodiments, the polypeptide encoded by the nucleotide sequence (1) comprises an amino acid sequence having at least 90% sequence identity with the polypeptide of SEQ ID NO: 1. According to certain embodiments, the polypeptide encoded by the nucleotide sequence (1) comprises an amino acid sequence having at least 95% sequence identity with the polypeptide of SEQ ID NO: 1. According to certain embodiments, the polypeptide encoded by the nucleotide sequence (1) consists of the amino acid sequence of SEQ ID NO: 1.

Suitably, the polypeptide encoded by the nucleotide sequence (1) has the same functional property as the polypeptide of SEQ ID NO: 1 [ChdP]. ChdP is a ketosynthase-alpha. The N terminal catalytic domain of the ChdP protein harbours a well conserved aa region around the highly conserved active site Cys173 (GPVGLVSTGCTSGVDVIGHA) responsible for catalyzing the iterative condensation of the ketoacyl:ACP intermediates. In the C terminus of the protein there is an amino-acid sequence characteristic of the acyltransferase site (VPVSSIKSMVGHSLGAIGSLEVAA) with the active Ser351 residue that binds to an acyl chain (Fernandez-Moreno et al. 1992). Specifically, ChdP catalyses a Claisen-type C—C bond formation from CoA activated acyl building blocks leading to the formation of a 20-carbon decaketide. Accordingly, the polypeptide encoded by the nucleotide sequence (1) has ketosynthase-alpha activity.

According to certain embodiments, the polypeptide encoded by the nucleotide sequence (2) comprises an amino acid sequence having at least 85% sequence identity with the polypeptide of SEQ ID NO: 2. According to certain embodiments, the polypeptide encoded by the nucleotide sequence (2) comprises an amino acid sequence having at least 90% sequence identity with the polypeptide of SEQ ID NO: 2. According to certain embodiments, the polypeptide encoded by the nucleotide sequence (2) comprises an amino acid sequence having at least 95% sequence identity with the polypeptide of SEQ ID NO: 2. According to certain embodiments, the polypeptide encoded by the nucleotide sequence (2) consists of the amino acid sequence of SEQ ID NO: 2.

Suitably, the polypeptide encoded by the nucleotide sequence (2) has the same functional property as the polypeptide of SEQ ID NO: 2 [ChdK]. ChdK is a ketosynthase-beta (KSβ), also called chain-length factor. Ketosynthase domain active-site cysteine residue in ChdK is replaced by a highly conserved glutamine as in KSQ (VSEQ¹⁷⁵AGGLD) and in other chain-length factors of type II PKS synthases. Specifically, ChdK is acting together with ketosynthase-alpha in the formation of the 20-carbon decaketide. Accordingly, the polypeptide encoded by the nucleotide sequence (2) has ketosynthase-beta activity.

According to certain embodiments, the polypeptide encoded by the nucleotide sequence (3) comprises an amino acid sequence having at least 85% sequence identity with the polypeptide of SEQ ID NO: 3. According to certain embodiments, the polypeptide encoded by the nucleotide sequence (3) comprises an amino acid sequence having at least 90% sequence identity with the polypeptide of SEQ ID NO:3. According to certain embodiments, the polypeptide encoded by the nucleotide sequence (3) comprises an amino acid sequence having at least 95% sequence identity with the polypeptide of SEQ ID NO: 3. According to certain embodiments, the polypeptide encoded by the nucleotide sequence (3) consists of the amino acid sequence of SEQ ID NO: 3.

Suitably, the polypeptide encoded by the nucleotide sequence (3) has the same functional property as the polypeptide of SEQ ID NO: 3 [ChdS]. ChdS is an acyl carrier protein which harbours an active Ser41 residue in the highly conserved motif (LGYDSL), to which phosphopantetheine binds in order to connect the incoming extender unit (Walsh et al. 1997). Specifically, ChdS cooperates with ketosynthase-alpha and ketosynthase-beta in the formation of the 20-carbon decaketide by serving as an anchor for the growing polyketide chain. Accordingly, the polypeptide encoded by the nucleotide sequence (3) is capable of acting as acyl carrier protein (ACP).

According to certain embodiments, the polypeptide encoded by the nucleotide sequence (4) comprises an amino acid sequence having at least 85% sequence identity with the polypeptide of SEQ ID NO: 4. According to certain embodiments, the polypeptide encoded by the nucleotide sequence (4) comprises an amino acid sequence having at least 90% sequence identity with the polypeptide of SEQ ID NO: 4. According to certain embodiments, the polypeptide encoded by the nucleotide sequence (4) comprises an amino acid sequence having at least 95% sequence identity with the polypeptide of SEQ ID NO: 4. According to certain embodiments, the polypeptide encoded by the nucleotide sequence (4) consists of the amino acid sequence of SEQ ID NO: 4.

Suitably, the polypeptide encoded by the nucleotide sequence (4) has the same functional property as the polypeptide of SEQ ID NO: 4 [ChdQI]. ChdQI is a bifunctional cyclase/aromatase. Within ChdQI there are the highly conserved amino acids, which are, according to the homologous cyclase/aromatase BexL, responsible for the determination of the final length of the polyketide and for its proper regiospecific cyclisation and aromatization (Ames et al. 2008). These amino acids are at positions Trp-32, Trp-68, Ser-70, Arg-72, and Trp-99. Specifically, ChdQI together with ChdQII catalyses the dehydration of the C9-hydroxyl and the subsequent aromatisation of the first ring (D). Accordingly, the polypeptide encoded by the nucleotide sequence (4) has cyclase/aromatase activity.

According to certain embodiments, the polypeptide encoded by the nucleotide sequence (5) comprises an amino acid sequence having at least 85% sequence identity with the polypeptide of SEQ ID NO: 5. According to certain embodiments, the polypeptide encoded by the nucleotide sequence (5) comprises an amino acid sequence having at least 90% sequence identity with the polypeptide of SEQ ID NO: 5. According to certain embodiments, the polypeptide encoded by the nucleotide sequence (5) comprises an amino acid sequence having at least 95% sequence identity with the polypeptide of SEQ ID NO: 5. According to certain embodiments, the polypeptide encoded by the nucleotide sequence (5) consists of the amino acid sequence of SEQ ID NO: 5.

Suitably, the polypeptide encoded by the nucleotide sequence (5) has the same functional property as the polypeptide of SEQ ID NO: 5 [ChdQII]. ChdQII is a bifunctional cyclase/aromatase. Similarly as in the case of ChdQI, there are the highly conserved amino acids at positions Trp-32, Phe-36, Trp-67, Ser-69, Arg-71, Met-94 and Trp-98. Specifically, ChdQII catalyses together with ChdQI the dehydration of the C-9 hydroxyl and the subsequent aromatisation of the first ring (D). Accordingly, the polypeptide encoded by the nucleotide sequence (5) has cyclase/aromatase activity.

According to certain embodiments, the polypeptide encoded by the nucleotide sequence (6) comprises an amino acid sequence having at least 85% sequence identity with the polypeptide of SEQ ID NO: 6. According to certain embodiments, the polypeptide encoded by the nucleotide sequence (6) comprises an amino acid sequence having at least 90% sequence identity with the polypeptide of SEQ ID NO: 6. According to certain embodiments, the polypeptide encoded by the nucleotide sequence (6) comprises an amino acid sequence having at least 95% sequence identity with the polypeptide of SEQ ID NO: 6. According to certain embodiments, the polypeptide encoded by the nucleotide sequence (6) consists of the amino acid sequence of SEQ ID NO: 6.

Suitably, the polypeptide encoded by the nucleotide sequence (6) has the same functional property as the polypeptide of SEQ ID NO: 6 [ChdX]. ChdX is a cyclase. Specifically, ChdX catalyses aldol condensation between C-1 and C-18, resulting in formation of the fourth ring (A). Accordingly, the polypeptide encoded by the nucleotide sequence (6) has cyclase activity.

According to certain embodiments, the polypeptide encoded by the nucleotide sequence (7) comprises an amino acid sequence having at least 85% sequence identity with the polypeptide of SEQ ID NO: 7. According to certain embodiments, the polypeptide encoded by the nucleotide sequence (7) comprises an amino acid sequence having at least 90% sequence identity with the polypeptide of SEQ ID NO: 7. According to certain embodiments, the polypeptide encoded by the nucleotide sequence (7) comprises an amino acid sequence having at least 95% sequence identity with the polypeptide of SEQ ID NO: 7. According to certain embodiments, the polypeptide encoded by the nucleotide sequence (7) consists of the amino acid sequence of SEQ ID NO: 7.

Suitably, the polypeptide encoded by the nucleotide sequence (7) has the same functional property as the polypeptide of SEQ ID NO: 7 [ChdL]. ChdL is an acyl-CoA ligase. Specifically, ChdL activates carboxylic acids as CoA thioesters. Accordingly, the polypeptide encoded by the nucleotide sequence (7) has acyl-CoA ligase activity.

According to certain embodiments, the polypeptide encoded by the nucleotide sequence (8) comprises an amino acid sequence having at least 85% sequence identity with the polypeptide of SEQ ID NO: 8. According to certain embodiments, the polypeptide encoded by the nucleotide sequence (8) comprises an amino acid sequence having at least 90% sequence identity with the polypeptide of SEQ ID NO: 8. According to certain embodiments, the polypeptide encoded by the nucleotide sequence (8) comprises an amino acid sequence having at least 95% sequence identity with the polypeptide of SEQ ID NO: 8. According to certain embodiments, the polypeptide encoded by the nucleotide sequence (8) consists of the amino acid sequence of SEQ ID NO: 8.

Suitably, the polypeptide encoded by the nucleotide sequence (8) has the same functional property as the polypeptide of SEQ ID NO: 8 [ChdT]. ChdT is a ketoreductase. Two conserved domains can be found within the amino acid sequence of ChdT proposed to act as a NADPH-cofactor binding sites (Hopwood, Sherman 1990; Rawlings, Cronan, J. E., Jr. 1992). Specifically, ChdT regiospecifically cyclizes the linear poly-beta-ketone from C-12 to C-7, followed by a C-9-carbonyl reduction. Accordingly, the polypeptide encoded by the nucleotide sequence (8) has ketoreductase activity.

According to certain embodiments, the polypeptide encoded by the nucleotide sequence (9) comprises an amino acid sequence having at least 85% sequence identity with the polypeptide of SEQ ID NO: 9. According to certain embodiments, the polypeptide encoded by the nucleotide sequence (9) comprises an amino acid sequence having at least 90% sequence identity with the polypeptide of SEQ ID NO: 9. According to certain embodiments, the polypeptide encoded by the nucleotide sequence (9) comprises an amino acid sequence having at least 95% sequence identity with the polypeptide of SEQ ID NO: 9. According to certain embodiments, the polypeptide encoded by the nucleotide sequence (9) consists of the amino acid sequence of SEQ ID NO: 9.

Suitably, the polypeptide encoded by the nucleotide sequence (9) has the same functional property as the polypeptide of SEQ ID NO: 9 [ChdMI]. ChdMI is a S-adenosylmethionine (SAM)-dependent C-6 methyltransferase. Specifically, ChdMI catalyses the methylation of C-6. Accordingly, the polypeptide encoded by the nucleotide sequence (9) has S-adenosylmethionine (SAM)-dependent C-6 methyltransferase activity.

According to certain embodiments, the polypeptide encoded by the nucleotide sequence (10) comprises an amino acid sequence having at least 85% sequence identity with the polypeptide of SEQ ID NO: 10. According to certain embodiments, the polypeptide encoded by the nucleotide sequence (10) comprises an amino acid sequence having at least 90% sequence identity with the polypeptide of SEQ ID NO: 10. According to certain embodiments, the polypeptide encoded by the nucleotide sequence (10) comprises an amino acid sequence having at least 95% sequence identity with the polypeptide of SEQ ID NO: 10. According to certain embodiments, the polypeptide encoded by the nucleotide sequence (10) consists of the amino acid sequence of SEQ ID NO: 10.

Suitably, the polypeptide encoded by the nucleotide sequence (10) has the same functional property as the polypeptide of SEQ ID NO: 10 [ChdMII]. ChdMII is S-adenosylmethionine-dependent (SAM)C-9 methyltransferase. Specifically, ChdMII catalyses the methylation of C-9. Similarly as for ChdMI, ChdMII also shows a typical glycine-rich SAM-dependent methyltransferase motif that interacts with the SAM cofactor, which is used as a source for the methyl group (Martin, McMillan 2002). Accordingly, the polypeptide encoded by the nucleotide sequence (10) has S-adenosylmethionine (SAM)-dependent methyltransferase activity.

According to certain embodiments, the polypeptide encoded by the nucleotide sequence (11) comprises an amino acid sequence having at least 85% sequence identity with the polypeptide of SEQ ID NO: 11. According to certain embodiments, the polypeptide encoded by the nucleotide sequence (11) comprises an amino acid sequence having at least 90% sequence identity with the polypeptide of SEQ ID NO: 11. According to certain embodiments, the polypeptide encoded by the nucleotide sequence (11) comprises an amino acid sequence having at least 95% sequence identity with the polypeptide of SEQ ID NO: 11. According to certain embodiments, the polypeptide encoded by the nucleotide sequence (11) consists of the amino acid sequence of SEQ ID NO: 11.

Suitably, the polypeptide encoded by the nucleotide sequence (11) has the same functional property as the polypeptide of [ChdN]. ChdN is a pyridoxal 5′-phosphate-dependent aminotransferase. Specifically, ChdN catalyses the single amination at the C-4. Accordingly, the polypeptide encoded by the nucleotide sequence (11) has pyridoxal 5′-phosphate-dependent aminotransferase activity.

According to certain embodiments, the polypeptide encoded by the nucleotide sequence (12) comprises an amino acid sequence having at least 85% sequence identity with the polypeptide of SEQ ID NO: 12. According to certain embodiments, the polypeptide encoded by the nucleotide sequence (12) comprises an amino acid sequence having at least 90% sequence identity with the polypeptide of SEQ ID NO: 12. According to certain embodiments, the polypeptide encoded by the nucleotide sequence (12) comprises an amino acid sequence having at least 95% sequence identity with the polypeptide of SEQ ID NO: 12. According to certain embodiments, the polypeptide encoded by the nucleotide sequence (12) consists of the amino acid sequence of SEQ ID NO: 12.

Suitably, the polypeptide encoded by the nucleotide sequence (12) has the same functional property as the polypeptide of SEQ ID NO: 12 [ChdGIV]. ChdGIV is a glycosyltransferase. Accordingly, the polypeptide encoded by the nucleotide sequence (12) has glycosyltransferase activity.

According to certain embodiments, the polypeptide encoded by the nucleotide sequence (13) comprises an amino acid sequence having at least 85% sequence identity with the polypeptide of SEQ ID NO: 13. According to certain embodiments, the polypeptide encoded by the nucleotide sequence (13) comprises an amino acid sequence having at least 90% sequence identity with the polypeptide of SEQ ID NO: 13. According to certain embodiments, the polypeptide encoded by the nucleotide sequence (13) comprises an amino acid sequence having at least 95% sequence identity with the polypeptide of SEQ ID NO: 13. According to certain embodiments, the polypeptide encoded by the nucleotide sequence (13) consists of the amino acid sequence of SEQ ID NO: 13.

Suitably, the polypeptide encoded by the nucleotide sequence (13) has the same functional property as the polypeptide of SEQ ID NO: 13 [ChdTn]. ChdTn is a transposase. Accordingly, the polypeptide encoded by the nucleotide sequence (13) has transposase activity.

According to certain embodiments, the polypeptide encoded by the nucleotide sequence (14) comprises an amino acid sequence having at least 85% sequence identity with the polypeptide of SEQ ID NO: 14. According to certain embodiments, the polypeptide encoded by the nucleotide sequence (14) comprises an amino acid sequence having at least 90% sequence identity with the polypeptide of SEQ ID NO: 14. According to certain embodiments, the polypeptide encoded by the nucleotide sequence (14) comprises an amino acid sequence having at least 95% sequence identity with the polypeptide of SEQ ID NO: 14. According to certain embodiments, the polypeptide encoded by the nucleotide sequence (14) consists of the amino acid sequence of SEQ ID NO: 14.

Suitably, the polypeptide encoded by the nucleotide sequence (14) has the same functional property as the polypeptide of SEQ ID NO: 14 [ChdR]. ChdR is an exporter from the EmrB/QacA subfamily. Specifically, ChdR is an integral membrane protein facilitating the efflux of chelocardin from a cell. Accordingly, the polypeptide encoded by the nucleotide sequence (14) is capable of acting as an exporter.

According to certain embodiments, the polypeptide encoded by the nucleotide sequence (15) comprises an amino acid sequence having at least 85% sequence identity with the polypeptide of SEQ ID NO: 15. According to certain embodiments, the polypeptide encoded by the nucleotide sequence (15) comprises an amino acid sequence having at least 90% sequence identity with the polypeptide of SEQ ID NO: 15. According to certain embodiments, the polypeptide encoded by the nucleotide sequence (15) comprises an amino acid sequence having at least 95% sequence identity with the polypeptide of SEQ ID NO: 15. According to certain embodiments, the polypeptide encoded by the nucleotide sequence (15) consists of the amino acid sequence of SEQ ID NO: 15.

Suitably, the polypeptide encoded by the nucleotide sequence (15) has the same functional property as the polypeptide of SEQ ID NO: 15 [ChdA]. ChdA is a transcriptional regulator most similar to the tetracycline repressor from the TetR family of proteins that are involved in the transcriptional control of multidrug efflux pumps. Specifically, ChdA regulates the expression of the exporter ChdR. Accordingly, the polypeptide encoded by the nucleotide sequence (15) has transcriptional regulator activity.

According to certain embodiments, the polypeptide encoded by the nucleotide sequence (16) comprises an amino acid sequence having at least 85% sequence identity with the polypeptide of SEQ ID NO: 16. According to certain embodiments, the polypeptide encoded by the nucleotide sequence (16) comprises an amino acid sequence having at least 90% sequence identity with the polypeptide of SEQ ID NO: 16. According to certain embodiments, the polypeptide encoded by the nucleotide sequence (16) comprises an amino acid sequence having at least 95% sequence identity with the polypeptide of SEQ ID NO: 16. According to certain embodiments, the polypeptide encoded by the nucleotide sequence (16) consists of the amino acid sequence of SEQ ID NO: 16.

Suitably, the polypeptide encoded by the nucleotide sequence (16) has the same functional property as the polypeptide of SEQ ID NO: 16 [ChdOI]. ChdOI is a FAD-dependent oxygenase. ChdOI possesses at the N-terminal end a typical conserved sequence G-X-G-2X-G-3X-A-6X-G (where X is any naturally occurring amino acid) which is involved in the FAD-cofactor binding (Mason, Cammack 1992). Specifically, ChdOI catalyses the hydroxylation of C-4. Accordingly, the polypeptide encoded by the nucleotide sequence (16) has a FAD-dependent oxygenase activity.

According to certain embodiments, the polypeptide encoded by the nucleotide sequence (17) comprises an amino acid sequence having at least 85% sequence identity with the polypeptide of SEQ ID NO: 17. According to certain embodiments, the polypeptide encoded by the nucleotide sequence (17) comprises an amino acid sequence having at least 90% sequence identity with the polypeptide of SEQ ID NO: 17. According to certain embodiments, the polypeptide encoded by the nucleotide sequence (17) comprises an amino acid sequence having at least 95% sequence identity with the polypeptide of SEQ ID NO: 17. According to certain embodiments, the polypeptide encoded by the nucleotide sequence (17) consists of the amino acid sequence of SEQ ID NO: 17.

Suitably, the polypeptide encoded by the nucleotide sequence (17) has the same functional property as the polypeptide of SEQ ID NO: 17 [ChdOIII]. ChdOIII is an ABM (Antibiotic Biosynthesis Monooxygenase) which catalyses molecular oxygen activation. Accordingly, the polypeptide encoded by the nucleotide sequence (17) has monooxygenase activity.

According to certain embodiments, the polypeptide encoded by the nucleotide sequence (18) comprises an amino acid sequence having at least 85% sequence identity with the polypeptide of SEQ ID NO: 18. According to certain embodiments, the polypeptide encoded by the nucleotide sequence (18) comprises an amino acid sequence having at least 90% sequence identity with the polypeptide of SEQ ID NO: 18. According to certain embodiments, the polypeptide encoded by the nucleotide sequence (18) comprises an amino acid sequence having at least 95% sequence identity with the polypeptide of SEQ ID NO: 18. According to certain embodiments, the polypeptide encoded by the nucleotide sequence (18) consists of the amino acid sequence of SEQ ID NO: 18.

Suitably, the polypeptide encoded by the nucleotide sequence (18) has the same functional property as the polypeptide of SEQ ID NO: 18 [ChdOII]. ChdOII is a FAD-dependent oxygenase. Specifically, ChdOII catalyses the hydroxylation of C-4 and C-12a. Like ChdOI, ChdOII possesses at the N-terminal end the typical conserved sequence G-X-G-2X-G-3X-A-6X-G (where X is any naturally occurring amino acid) which is involved in the FAD-cofactor binding (Mason, Cammack 1992). Accordingly, the polypeptide encoded by the nucleotide sequence (18) has FAD-dependent oxygenase activity.

According to certain embodiments, the polypeptide encoded by the nucleotide sequence (19) comprises an amino acid sequence having at least 85% sequence identity with the polypeptide of SEQ ID NO: 19. According to certain embodiments, the polypeptide encoded by the nucleotide sequence (19) comprises an amino acid sequence having at least 90% sequence identity with the polypeptide of SEQ ID NO: 19. According to certain embodiments, the polypeptide encoded by the nucleotide sequence (19) comprises an amino acid sequence having at least 95% sequence identity with the polypeptide of SEQ ID NO: 19. According to certain embodiments, the polypeptide encoded by the nucleotide sequence (19) consists of the amino acid sequence of SEQ ID NO: 19.

Suitably, the polypeptide encoded by the nucleotide sequence (19) has the same functional property as the polypeptide of SEQ ID NO: 19 [ChdY]. ChdY is a cyclase, containing a conserved motif HXGTHXDXPXH (where X is any naturally occurring amino acid), characteristic of cyclase family PF04199 that is likely to form part of the active site. Specifically, ChdY catalyses an aldol condensation between C-5 and C-14 which results in the cyclization of the second ring (C). Accordingly, the polypeptide encoded by the nucleotide sequence (19) has cyclase activity.

As noted above, the present inventors have also identified two so far undiscovered regulatory genes within the chelocardin biosynthetic gene cluster of the wild-type chelocardin producer A. sulphurea which encode transcriptional activators belonging to the SARP and LuxR family, respectively. While these regulatory genes are not directly involved in the synthesis of chelocardin, they are expected to have a positive effect on the production chelocardin in a heterologous host as seen for homologous family members in oxytetracycline production.

Therefore, the gene cluster of the present invention may further comprise at least one of the nucleotide sequences (20) and (21):

(20) a nucleotide sequence encoding a polypeptide which comprises an amino acid sequence having at least 80%, such as at least 85%, sequence identity with the polypeptide of SEQ ID NO: 20 [SARP/ChdB]; and

(21) a nucleotide sequence encoding a polypeptide which comprises an amino acid sequence having at least 80%, such as at least 85%, sequence identity with the polypeptide of SEQ ID NO: 21 [LuxR/ChdC].

According to certain embodiments, the gene cluster comprises the nucleotide sequence (20).

According to certain embodiments, the gene cluster comprises the nucleotide sequence (21).

According to certain embodiments, the gene cluster comprises both nucleotide sequences (20) and (21).

According to certain embodiments, the polypeptide encoded by the nucleotide sequence (20) comprises an amino acid sequence having at least 85% sequence identity with the polypeptide of SEQ ID NO: 20. According to certain embodiments, the polypeptide encoded by the nucleotide sequence (20) comprises an amino acid sequence having at least 90% sequence identity with the polypeptide of SEQ ID NO: 20. According to certain embodiments, the polypeptide encoded by the nucleotide sequence (20) comprises an amino acid sequence having at least 95% sequence identity with the polypeptide of SEQ ID NO: 20. According to certain embodiments, the polypeptide encoded by the nucleotide sequence (20) consists of the amino acid sequence of SEQ ID NO: 20.

Suitably, the polypeptide encoded by the nucleotide sequence (20) has the same functional property as the polypeptide of SEQ ID NO: 20 [ChdB]. ChdB is a transcriptional activator belonging to the family of Streptomyces antibiotic regulatory protein (SARP) family. It is homologous to OtcR, identified by Yin et al. (Yin et al. 2015), which acts as a positive pathway-specific activator of OTC biosynthesis leading to a significant increase in OTC production when overexpressed at the appropriate level. Accordingly, the polypeptide encoded by the nucleotide sequence (20) has transcriptional activator activity.

According to certain embodiments, the polypeptide encoded by the nucleotide sequence (21) comprises an amino acid sequence having at least 85% sequence identity with the polypeptide of SEQ ID NO: 21. According to certain embodiments, the polypeptide encoded by the nucleotide sequence (21) comprises an amino acid sequence having at least 90% sequence identity with the polypeptide of SEQ ID NO: 21. According to certain embodiments, the polypeptide encoded by the nucleotide sequence (21) comprises an amino acid sequence having at least 95% sequence identity with the polypeptide of SEQ ID NO: 21. According to certain embodiments, the polypeptide encoded by the nucleotide sequence (21) consists of the amino acid sequence of SEQ ID NO: 21.

Suitably, the polypeptide encoded by the nucleotide sequence (21) has the same functional property as the polypeptide of SEQ ID NO: 21 [ChdC]. ChdC is a transcriptional activator with high similarity to transcriptional activators of the LuxR family, containing a conserved C-terminal helix-turn-helix (HTH) motif, characteristic of LuxR family (PROSITE PS00622). It is homologous to the regulatory protein OtcG from OTC biosynthesis, identified by Lešnik et al. (Lesnik et al. 2009)(Lesnik et al. 2015) and shown to have a conditionally positive role in OTC biosynthesis: its inactivation reduced the production of OTC by more than 40%. Accordingly, the polypeptide encoded by the nucleotide sequence (21) has transcriptional activator activity.

The various polypeptides encoded by the gene cluster of the present invention are summarized in Table 1 below.

TABLE 1
Genes of the gene cluster of the present invention

| SEQ ID | | |
|---|---|---|
| NO: | Gene Name | Functional property |
| 1 | chdP | Ketosynthase - alpha |
| 2 | chdK | Ketosynthase - beta |
| 3 | chdS | Acyl Carrier Protein |
| 4 | chdQI | Cyclase/Aromatase |
| 5 | chdQII | Cyclase/Aromatase |
| 6 | chdX | Cyclase |
| 7 | chdL | Acyl-CoA Ligase |
| 8 | chdT | Ketoreductase |
| 9 | chdMI | Methyltransferase |
| 10 | chdMIl | Methyltransferase |
| 11 | chdN | Aminotransferase |
| 12 | chdGIV | Glycosyltransferase |
| 13 | chdTn | Transposase |
| 14 | chdR | Exporter |
| 15 | chdA | Transcriptional Regulator |
| 16 | chdOI | Oxygenase |
| 17 | chdOIII | Oxygenase |
| 18 | chdOII | Oxygenase |
| 19 | chdY | Cyclase |
| 20 | chdB | Transcriptional Activator |
| 21 | chdC | Transcriptional Activator |

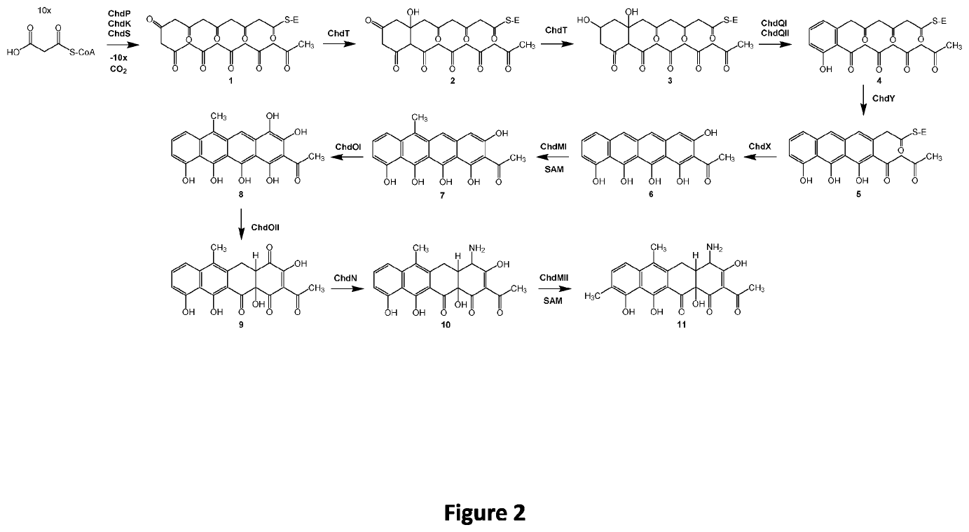

The chelocardin biosynthetic pathway involving the above described polypeptide is shown in FIG. 2. The polyketide skeleton of chelocardin is assembled from an acetate starter unit to which 9 malonate-derived acetate building blocks are attached by the action of the minimal PKS, namely ChdP, ChdK, ChdS. The polyketide chain is further subjected to C-9 ketoreduction and cyclisation/aromatisation, by the action of the ChdT ketoreductase, and the two cyclases/aromatases, ChdQII and ChdQI, respectively. After the cyclisation is completed by ChdY and ChdX, the nascent aromatic compound is subjected to post-PKS reactions, i.e. C-6 methylation, oxidations, C-4 amination, and C-9 methylation, catalysed by ChdMI methyltransferase, three oxygenases ChdOI/ChdOII/ChdOIII, aminotransferase ChdN, and methyltransferase ChdMII, respectively.

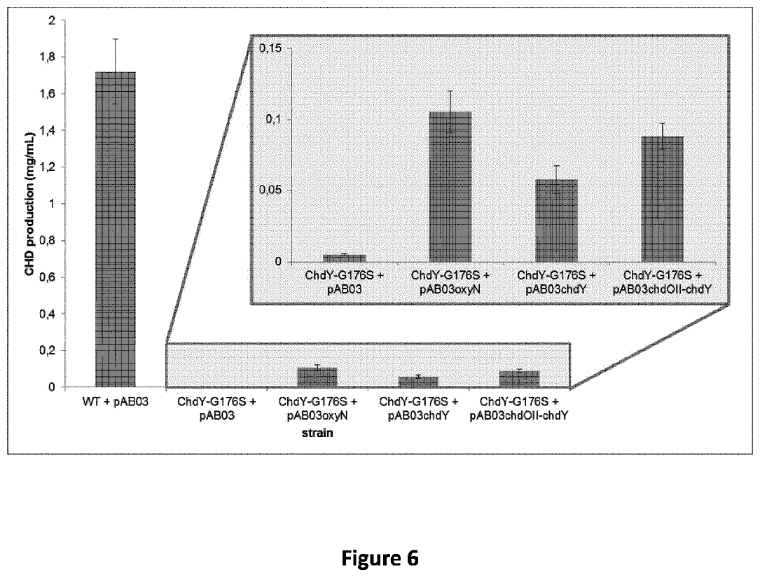

Further, sequence analysis of genomic DNA isolated from the wild-type chelocardin producer A. sulphurea has surprisingly revealed that ChdOII and ChdY, which are arranged in a successive order within the naturally occurring gene cluster, are expressed in the form of a fusion protein due to the absence of a stop codon at the end of the ChdOII encoding gene. Mutagenesis and complementation experiments have shown that the ChdOII and ChdY activities can be decoupled (FIG. 6). Accordingly, each of ChdOII and ChdY can be expressed as individual polypeptides or as a fusion protein.

Therefore, according to certain embodiments, the nucleotide sequences (18) and (19) are linked in the gene cluster to form a fusion protein of the respective polypeptides encoded by them. The so formed fusion protein may thus comprise an amino acid sequence having at least 80%, such as at least 85%, sequence identity with the polypeptide of SEQ ID NO: 22 and has the same functional properties as the polypeptides of SEQ ID NO: 18 and SEQ ID NO: 19 [ChdOII+ChdY]. Respective details are given above.

According to certain embodiments, the polypeptide encoded by the nucleotide sequence (22) comprises an amino acid sequence having at least 85% sequence identity with the polypeptide of SEQ ID NO: 22. According to certain embodiments, the polypeptide encoded by the nucleotide sequence (22) comprises an amino acid sequence having at least 90% sequence identity with the polypeptide of SEQ ID NO: 22. According to certain embodiments, the polypeptide encoded by the nucleotide sequence (22) comprises an amino acid sequence having at least 95% sequence identity with the polypeptide of SEQ ID NO: 22. According to certain embodiments, the polypeptide encoded by the nucleotide sequence (22) consists of the amino acid sequence of SEQ ID NO: 22.

In this respect, the present invention provides a (isolated) fusion protein comprises an amino acid sequence having at least 80%, such as 85%, sequence identity with the polypeptide of SEQ ID NO: 22, and a nucleic acid molecule comprising a nucleotide sequence encoding same. Suitably, said fusion protein has FAD-dependent oxygenase and cyclase activities.

According to certain embodiments, the fusion protein comprises an amino acid sequence having at least 85% sequence identity with the polypeptide of SEQ ID NO: 22. According to certain embodiments, the fusion protein comprises an amino acid sequence having at least 90% sequence identity with the polypeptide of SEQ ID NO: 22. According to certain embodiments, the fusion protein comprises an amino acid sequence having at least 95% sequence identity with the polypeptide of SEQ ID NO: 22. According to certain embodiments, the fusion protein consists of the amino acid sequence of SEQ ID NO: 22.

Sequence analysis of genomic DNA isolated from the wild-type chelocardin producer A. sulphurea has furthermore confirmed that ChdL and ChdOIII, which are arranged in a successive order within the naturally occurring gene cluster, are also expressed in the form of a fusion protein due to the absence of a stop codon at the end of the ChdL encoding gene.

Therefore, according to certain embodiments, the nucleotide sequences (7) and (17) are linked in the gene cluster to form a fusion protein of the respective polypeptides encoded by them. The so formed fusion protein may thus comprise an amino acid sequence having at least 80%, such as at least 85%, sequence identity with the polypeptide of SEQ ID NO: 23 and has the same functional properties as the polypeptides of SEQ ID NO: 7 and SEQ ID NO: 17 [ChdL+ChdOIII]. Respective details are given above.

In this respect, the present invention provides a (isolated) fusion protein comprises an amino acid sequence having at least 80%, such as 85%, sequence identity with the polypeptide of SEQ ID NO: 23, and a nucleic acid molecule comprising a nucleotide sequence encoding same. Suitably, said fusion protein has acyl-CoA ligase and oxygenase activities.

According to certain embodiments, the fusion protein comprises an amino acid sequence having at least 85% sequence identity with the polypeptide of SEQ ID NO: 23. According to certain embodiments, the fusion protein comprises an amino acid sequence having at least 90% sequence identity with the polypeptide of SEQ ID NO: 23. According to certain embodiments, the fusion protein comprises an amino acid sequence having at least 95% sequence identity with the polypeptide of SEQ ID NO: 23. According to certain embodiments, the fusion protein consists of the amino acid sequence of SEQ ID NO: 23.

The polypeptide encoding nucleotide sequences comprised by the gene cluster of the present invention may be present in any order. In other words, the ordering of the polypeptide encoding nucleotide sequences in the gene cluster of the present invention may be the same or different from the naturally occurring order of polypeptide encoding nucleotide sequences within the gene cluster found in the wild-type chelocardin producer A. sulphurea.

A representative, non-limiting, nucleotide sequence of the CHD biosynthetic cluster found in the wild-type chelocardin producer A. sulphurea is presented in SEQ ID NO: 26 (including additional 100 bp upstream and 100 bp downstream of the actual cluster sequence).

The present invention further relates to a DNA construct comprising the gene cluster according to the present invention.

The DNA construct may comprise at least one genetic element for facilitating expression of the polypeptide encoding nucleotide sequences comprised by the gene cluster of the present invention, such as at least one promoter. Suitably, the at least one promoter is operably linked to the gene cluster.

Promoters useful in accordance with the invention are any known promoters that are functional in a given host cell to cause the production of an mRNA molecule. Many such promoters are known to the skilled person. Such promoters include promoters normally associated with other genes, and/or promoters isolated from any bacteria. The use of promoters for protein expression is generally known to those of skilled in the art of molecular biology, for example, see Sambrook et al. (Sambrook, Russell 2001). The promoter employed may be inducible, such as a temperature inducible promoter (e.g., a pL or pR phage lambda promoters, each of which can be controlled by the temperature-sensitive lambda repressor c1857). The term “inducible” used in the context of a promoter means that the promoter only directs transcription of an operably linked nucleotide sequence if a stimulus is present, such as a change in temperature or the presence of a chemical substance (“chemical inducer”). As used herein, “chemical induction” according to the present invention refers to the physical application of an exogenous or endogenous substance (incl. macromolecules, e.g., proteins or nucleic acids) to a host cell. This has the effect of causing the target promoter present in the host cell to increase the rate of transcription. Alternatively, the promoter employed may be constitutive. The term “constitutive” used in the context of a promoter means that the promoter is capable of directing transcription of an operably linked nucleotide sequence in the absence of stimulus (such as heat shock, chemicals etc.). Examples of promoters that have been commonly used to express heterologous polypeptides, include, without limitation, P_{ermE*} promoter, Pm promoter, lac promoter, trp promoter, tac promoter, λpL promoter, T7 promoter, phoA promoter, araC promoter, xapA promoter, cad promoter and recA promoter.

Besides a promoter, the DNA construct may further comprise at least one genetic element selected from a 5′ untranslated region (5′UTR) and 3′ untranslated region (3′ UTR). Many such 5′ UTRs and 3′ UTRs derived from prokaryotes are well known to the skilled person. Such genetic elements include 5′ UTRs and 3′ UTRs normally associated with other genes, and/or 5′ UTRs and 3′ UTRs isolated from any prokaryotes, notably bacteria. Usually, the 5′ UTR contains a ribosome binding site (RBS), also known as the Shine Dalgarno sequence which is usually 3-10 base pairs upstream from the initiation codon. The ribosome binding site may be an RBS naturally associated with a prokaryotic gene or may be synthetic.

Further genetic elements may include, but are not limited to, an enhancer, a response element, a terminator sequence, a polyadenylation sequence, and the like.

The DNA construct may be a vector, such as an expression vector, or part of a vector, such as an expression cassette. Normally, such a vector remains extrachromosomal within the host cell which means that it is found outside of the nucleus or nucleoid region of the cell. However, it is also contemplated by the present invention that the DNA construct is stably integrated into at least one chromosome of a host cell. Means for stable integration into a chromosome of a host cell, e.g., by homologous recombination, are well known to the skilled person. For example, the DNA construct may contain one or more integration elements facilitating the integration into the chromosome of a host cell.

According to certain embodiments, the DNA constructed is a vector, such as an expression vector. According to particular embodiments, the vector is a plasmid, such as a cosmid. The vector may be an integrative vector, such as an integrative plasmid.

According to certain embodiments, the DNA constructed is an expression cassette.

The DNA construct may further include additional genes useful in modifying the structure of chelocardin. For example, it has been shown in EP2154249 (Petkovic et al.) and Lešnik et al. (Lesnik et al. 2015), that the chelocardin analogue 2-carboxamido-2-deacetyl-chelocardin (CDCHD) can be produced in a modified version of the wild-type producer A. sulphurea by introducing and expressing genes oxyD from the S. rimosus OTC gene cluster (oxyD alone or in combination with oxyP).

Therefore, according to certain embodiments, the DNA construct further comprises at least one nucleotide sequence selected from the nucleotide sequences (24) and (25):

(24) a nucleotide sequence encoding a polypeptide which comprises an amino acid sequence having at least 80%, such as at least 85%, sequence identity with the polypeptide of SEQ ID NO: 24 [OxyD]; and

(25) a nucleotide sequence encoding a polypeptide which comprises an amino acid sequence having at least 80%, such as at least 85%, sequence identity with the polypeptide of SEQ ID NO: 25 [OxyP].

Suitably, the polypeptide encoded by any of the nucleotide sequences (24) and (25) has the same functional property as the polypeptide to which it refers.

According to certain embodiments, the DNA construct further comprises a nucleotide sequence encoding a polypeptide which comprises an amino acid sequence having at least 80%, such as at least 85%, sequence identity with the polypeptide of SEQ ID NO: 24 and which has the same functional property as the polypeptide of SEQ ID NO: 24 [OxyD].

According to certain embodiments, the polypeptide encoded by the nucleotide sequence (24) comprises an amino acid sequence having at least 85% sequence identity with the polypeptide of SEQ ID NO: 24. According to certain embodiments, the polypeptide encoded by the nucleotide sequence (24) comprises an amino acid sequence having at least 90% sequence identity with the polypeptide of SEQ ID NO: 24. According to certain embodiments, the polypeptide encoded by the nucleotide sequence (24) comprises an amino acid sequence having at least 95% sequence identity with the polypeptide of SEQ ID NO: 24. According to certain embodiments, the polypeptide encoded by the nucleotide sequence (24) consists of the amino acid sequence of SEQ ID NO: 24.

Suitably, the polypeptide encoded by the nucleotide sequence (24) has the same functional property as the polypeptide of SEQ ID NO: 24 [OxyD]. OxyD is an amidotransferase catalysing the amidation of the acetyl group at C2 in the chelocardin structure, resulting in a carboxyamido moiety (see, e.g., Lešnik et al., 2015). Accordingly, the polypeptide encoded by the nucleotide sequence (24) has amidotransferase activity.

According to certain embodiments, the DNA construct further comprises a nucleotide sequence encoding a polypeptide which comprises an amino acid sequence having at least 80%, such as at least 85%, sequence identity with the polypeptide of SEQ ID NO: 25 and which has the same functional property as the polypeptide of SEQ ID NO: 25 [OxyP].

According to certain embodiments, the polypeptide encoded by the nucleotide sequence (25) comprises an amino acid sequence having at least 85% sequence identity with the polypeptide of SEQ ID NO: 25. According to certain embodiments, the polypeptide encoded by the nucleotide sequence (25) comprises an amino acid sequence having at least 90% sequence identity with the polypeptide of SEQ ID NO: 25. According to certain embodiments, the polypeptide encoded by the nucleotide sequence (25) comprises an amino acid sequence having at least 95% sequence identity with the polypeptide of SEQ ID NO: 25. According to certain embodiments, the polypeptide encoded by the nucleotide sequence (25) consists of the amino acid sequence of SEQ ID NO: 25.

Suitably, the polypeptide encoded by the nucleotide sequence (25) has the same functional property as the polypeptide of SEQ ID NO: 25 [OxyP]. OxyP is an acyltransferase which suppresses priming by acetate by removing the competing acetyl units, leading to increase in proportion of CDCHD compared to CHD. Accordingly, the polypeptide encoded by the nucleotide sequence (25) has acyltransferase activity.

Any of the nucleotide sequences (24) and (25) may be under control of the same promoter as the nucleotide sequences of the gene cluster or under control of a different promoter.

Moreover, while the nucleotide sequences (24) and (25) are described to be comprised by the DNA construct, it is also contemplated by the present invention that any of these nucleotide sequences is/are included in the gene cluster described herein.

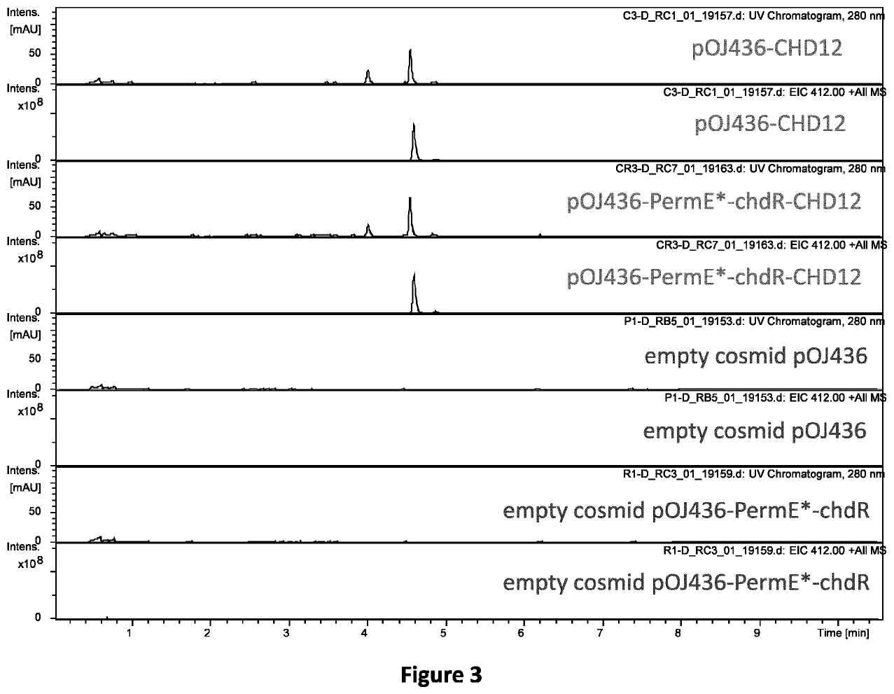

As further demonstrated herein, providing an additional copy of CHD efflux pump gene chdR improves self-resistance of a host cell during heterologous expression of CHD (FIG. 3). Particularly, an additional copy chdR led to slightly increased production yields of CHD up to approx. 60 mg/L.

Accordingly, the DNA construct of the present invention may further comprise an additional nucleotide sequence encoding a polypeptide which comprises an amino acid sequence having at least 80%, such as at least 85%, sequence identity with the polypeptide of SEQ ID NO: 14 and which has the same functional property as the polypeptide of SEQ ID NO: 14 [ChdR].

According to certain embodiments, the polypeptide encoded by the additional nucleotide sequence comprises an amino acid sequence having at least 85% sequence identity with the polypeptide of SEQ ID NO: 14. According to certain embodiments, the polypeptide encoded by the additional nucleotide sequence comprises an amino acid sequence having at least 90% sequence identity with the polypeptide of SEQ ID NO: 14. According to certain embodiments, the polypeptide encoded by the additional nucleotide sequence (14) comprises an amino acid sequence having at least 95% sequence identity with the polypeptide of SEQ ID NO: 14. According to certain embodiments, the polypeptide encoded by the nucleotide sequence (14) consists of the amino acid sequence of SEQ ID NO: 14.

The additional nucleotide sequences may be under control of the same promoter as the nucleotide sequences of the gene cluster or under control of a different promoter.

The present invention further provides a recombinant host cell comprising the gene cluster of the present invention or the DNA construct of the present invention, wherein the gene cluster or DNA construct is heterologous to said host cell. According to particular embodiments, said recombinant host cell heterologously expresses the polypeptides encoded by the gene cluster which allows for the biosynthesis of chelocardin or an analogue thereof.

The gene cluster or DNA construct may be extrachromosomal, e.g., in the form of a extrachromosomal vector, or it may be integrated into one or more chromosomes of said host cell. According to certain embodiments, the gene cluster or DNA construct is extrachromosomal. According to certain embodiments, the gene cluster of DNA constructed is integrated into one or more chromosomes of said host cell.

The recombinant host cell may further comprise at least one nucleotide sequence selected from the nucleotide sequences (24) and (25):

(24) a nucleotide sequence encoding a polypeptide which comprises an amino acid sequence having at least 80%, such as at least 85%, sequence identity with the polypeptide of SEQ ID NO: 24 and has the same functional property as the polypeptides of SEQ ID NO: 24 [OxyD]; and

(25) a nucleotide sequence encoding a polypeptide which comprises an amino acid sequence having at least 80%, such as at least 85%, sequence identity with the polypeptide of SEQ ID NO: 25 and has the same functional property as the polypeptides of SEQ ID NO: 25 [OxyP].

According to particular embodiments, the recombinant host cell heterologously expresses at least one polypeptide which comprises an amino acid sequence having at least 80%, such as at least 85%, sequence identity with the polypeptide of SEQ ID NO: 24 or 25 and which has the same functional property as the polypeptide of SEQ ID NO: 24 or 25, respectively.

According to certain embodiments, the recombinant host cell comprises a nucleotide sequence encoding a polypeptide which comprises an amino acid sequence having at least 80%, such as at least 85%, 90%, or 95%, sequence identity with the polypeptide of SEQ ID NO: 24 and has the same functional property as the polypeptide of SEQ ID NO: 24 [OxyD]. According to particular embodiments, the recombinant host cell heterologously expresses a polypeptide which comprises an amino acid sequence having at least 80%, such as at least 85%, such as at least 85%, 90%, or 95%, sequence identity with the polypeptide of SEQ ID NO: 24 and which has the same functional property as the polypeptide of SEQ ID NO: 24 [OxyD].

According to certain embodiments, the recombinant host cell comprises a nucleotide sequence encoding a polypeptide which comprises an amino acid sequence having at least 80%, such as at least 85%, such as at least 85%, 90%, or 95%, sequence identity with the polypeptide of SEQ ID NO: 25 and has the same functional property as the polypeptides of SEQ ID NO: 25 [OxyP]. According to particular embodiments, the recombinant host cell heterologously expresses a polypeptide which comprises an amino acid sequence having at least 80%, such as at least 8%, such as at least 85%, 90%, or 95%, sequence identity with the polypeptide of SEQ ID NO: 25 and which has the same functional property as the polypeptide of SEQ ID NO: 25 [OxyP].

The at least one nucleotide sequence selected from the nucleotide sequences (24) and (25) may be included in the gene cluster or DNA construct. However, it is also contemplated by the present invention that any of these nucleotide sequences is/are present in a further DNA construct, such as a further vector, which is different from the DNA construct comprising the gene cluster. Such further DNA construct may comprise at least one genetic element for facilitating expression of the polypeptide encoding nucleotide sequence(s) comprised thereby, such as those genetic elements detailed above, notably at least one promoter operably linked to the polypeptide encoding nucleotide sequence(s). Such further DNA construct may be extrachromosomal or integrated into one or more chromosomes of said host cell.

Therefore, according to certain embodiments, the recombinant host cell comprises a further (second) DNA construct comprising at least one nucleotide sequence selected from the nucleotide sequences (24) and (25) above.

The recombinant host cell may further comprise an additional nucleotide sequence encoding a polypeptide which comprises an amino acid sequence having at least 80%, such as at least 85%, 90% or 95%, sequence identity with the polypeptide of SEQ ID NO: 14 and which has the same functional property as the polypeptide of SEQ ID NO: 14 [ChdR].

The additional nucleotide sequence encoding a polypeptide comprises an amino acid sequence having at least 80% sequence identity with the polypeptide of SEQ ID NO: 14, may be included in the gene cluster or DNA construct. However, it is also contemplated by the present invention that the additional nucleotide sequence encoding a polypeptide comprises an amino acid sequence having at least 80% sequence identity with the polypeptide of SEQ ID NO: 14 is present in a further (second or third) DNA construct, such as a further vector, which is different from the DNA construct comprising the gene cluster. Such further DNA construct may comprise at least one genetic element for facilitating expression of the polypeptide encoding nucleotide sequence(s) comprised thereby, such as those genetic elements detailed above, notably at least one promoter operably linked to the polypeptide encoding nucleotide sequence(s). Such further DNA construct may be extrachromosomal or integrated into one or more chromosomes of said host cell.

Therefore, according to certain embodiments, the recombinant host cell comprises a further (second or third) DNA construct comprising a nucleotide sequence encoding a polypeptide which comprises an amino acid sequence having at least 80%, such as at least 85%, 90% or 95%, sequence identity with the polypeptide of SEQ ID NO: 14 and which has the same functional property as the polypeptide of SEQ ID NO: 14 [ChdR].

The recombinant host cell in accordance with the invention can be produced from any suitable host organism, including single-celled or multicellular microorganisms such as bacteria, yeast, fungi, algae and plant.

According to certain embodiments, a recombinant host cells in accordance is a prokaryotic organism, such as a bacterium.

According to certain embodiments, a recombinant host cells in accordance is a bacterium, such as a bacterium of the order Actinomycetales.

A bacterial host cells may be selected from Gram-positive and Gram-negative bacteria. Non-limiting examples for Gram-negative bacterial host cells include species from the genus Escherichia, such as Escherichia coli. Non-limiting examples of Gram-positive bacterial host cells include species from the genera Streptomyces, Amycolatopsis and Nocardia.

According to certain embodiments, the recombinant host cell is a bacterium belonging to a genus selected from the group consisting of Streptomyces, Amycolatopsis and Nocardia, such as a bacterium selected from the group consisting of Streptomyces lividans, Streptomyces coelicolor, Streptomyces albus, Streptomyces rimosus, Amycolatopsis mediterranei, Amycolatopsis orientalis and Nocardia spp.

According to certain embodiments, the recombinant host cell is Streptomyces lividans. According to certain embodiments, the recombinant host cell is Streptomyces coelicolor. According to certain embodiments, the recombinant host cell is Streptomyces albus. According to certain embodiments, the recombinant host cell is Streptomyces rimosus. According to certain embodiments, the recombinant host cell is Amycolatopsis mediterranei, According to certain embodiments, the recombinant host cell is Amycolatopsis orientalis. According to certain embodiments, the recombinant host cell is Nocardia spp.

The present invention further provides a process for the biosynthetic production of a tetracycline (notably chelocardin or an analogue thereof), said process comprises the steps of a) cultivating a recombinant host cell as described herein in the presence of a suitable substrate, such as a fermentable carbon substrate, under conditions conducive to the production of said tetracycline and, optionally, b) recovering the tetracycline from the cultivation medium employed in cultivation.

The medium employed may be any conventional medium suitable for culturing the host cell in question, and may be composed according to the principles of the prior art. The medium will usually contain all nutrients necessary for the growth and survival of the respective host cell, such as carbon and nitrogen sources and other inorganic salts. Suitable media, e.g. minimal or complex media, are available from commercial suppliers, or may be prepared according to published receipts, e.g. the American Type Culture Collection (ATCC) Catalogue of strains. Non-limiting standard medium well known to the skilled person include Luria Bertani (LB) broth, Sabouraud Dextrose (SD) broth, MS broth, Yeast Peptone Dextrose, BMMY, GMMY, or Yeast Malt Extract (YM) broth, which are all commercially available. A non-limiting example of suitable media for culturing bacterial cells, such as B. subtilis, L. lactis or E. coli cells, including minimal media and rich media such as Luria Broth (LB), M9 media, M17 media, SA media, MOPS media, Terrific Broth, YT and others. Suitable media for culturing eukaryotic cells, such as yeast cells, are RPMI 1640, MEM, DMEM, all of which may be supplemented with serum and/or growth factors as required by the particular host cell being cultured. The medium for culturing eukaryotic cells may also be any kind of minimal media such as Yeast minimal media.

Suitable conditions for culturing the respective host cell are well known to the skilled person. Typically, the recombinant host cell is cultured at a temperature ranging from about 23 to about 60° C., such as from about 25 to about 40° C., such as at about 30 to about 37° C., such as about 30° C. The pH of the medium may range from pH 1.0 to pH 14.0, such as from about pH 1 to about pH 2, from about pH 4 to about pH 11, from about pH 5 to about pH 10, from about pH 6 to about pH 10, or from about pH 7 to about pH 9.5, e.g. at pH 6.0, pH pH 7.0, pH. 7.5, pH 8.0, pH 8.5, pH 9.0, pH 9.5, pH 10.0, pH 10.5 or pH 11.0.

The process may further comprise b) recovering the tetracycline from the cultivation medium. The tetracycline may be recovered by conventional method for isolation and purification chemical compounds from a medium. Well-known purification procedures include centrifugation or filtration, precipitation, and chromatographic methods such as e.g. ion exchange chromatography, gel filtration chromatography, etc.

The present invention also pertains to a tetracycline (notably chelocardin or an analogue thereof) produced by the foregoing process. Particularly, the present invention pertains to a tetracycline having structure I:

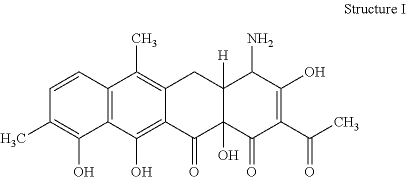 [SMILES: CC(=O)C1=C(O)C(N)C2Cc3c(c(O)c4c(O)c(C)ccc4c3C)C(=O)C2(O)C1=O]

optionally as a stereoisomer, including enantiomers and diastereomers, or in form of a mixture of at least two stereoisomers, including enantiomers and/or diastereomers, in any mixing ratio, or a corresponding salt thereof, or a corresponding solvate thereof.

According to certain embodiments, the compound of structure I is in the form of a stereoisomer. According to particular embodiments, the stereoisomer has the structure II

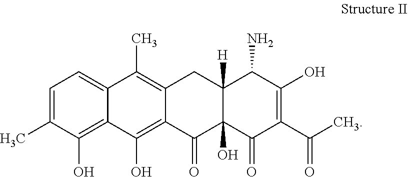 [SMILES: CC(=O)C1=C(O)[C@@H](N)[C@H]2Cc3c(c(O)c4c(O)c(C)ccc4c3C)C(=O)[C@@]2(O)C1=O]

The present invention further pertains to a tetracycline having structure III:

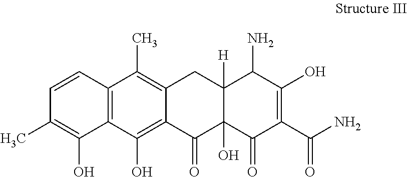 [SMILES: Cc1ccc2c(C)c3c(c(O)c2c1O)C(=O)C1(O)C(=O)C(C(N)=O)=C(O)C(N)C1C3]

optionally as a stereoisomer, including enantiomers and diastereomers, or in form of a mixture of at least two stereoisomers, including enantiomers and/or diastereomers, in any mixing ratio, or a corresponding salt thereof, or a corresponding solvate thereof.

According to certain embodiments, the compound of structure III is in the form of a stereoisomer. According to particular embodiments, the stereoisomer has the structure IV

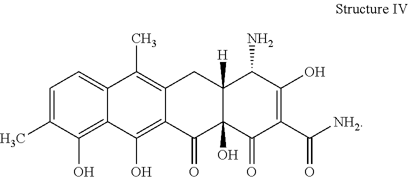 [SMILES: Cc1ccc2c(C)c3c(c(O)c2c1O)C(=O)[C@@]1(O)C(=O)C(C(N)=O)=C(O)[C@@H](N)[C@H]1C3]

The present invention further relates to the use a compound as described above as a medicament, such as in the treatment of a bacterial infection.

### Certain Definitions

“Gene cluster”, as used herein, shall be understood to be a totality of DNA coding for polypeptides required to catalyse a certain biochemical pathway. A gene cluster can be on a single DNA molecule, or can be on multiple DNA molecules, e.g. in form of a DNA library.

“Heterologous”, as used herein, means that a polynucleotide or polypeptide is normally not found in or made (i.e. expressed) by the host cell, but derived from a different organism or made synthetically. Moreover, a host cell transformed with a gene cluster or DNA construct described herein which is not normally present in the host cell would be considered heterologous for the purpose of the present invention.

“Host cell” as used herein refers to a living cell or microorganism that is capable of reproducing its genetic material and along with it recombinant genetic material that has been introduced into it—e.g., via heterologous transformation.

“Recombinant”, as used herein, with reference to, e.g., a host cell, polynucleotide, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant host cells expressing a gene or gene cluster that is not found within the native (non-recombinant) form of the cell or express native genes that are otherwise expressed at a different level.

“Isolated”, as used herein, means that a polynucleotide (such as the gene cluster of the present invention) or polypeptide (such as a fusion protein of the present invention) is removed from its original environment (e.g., the environment in which it naturally occurs). Particularly, a polynucleotide or polypeptide which has been separated from some or all of the coexisting materials in the natural system is considered isolated.

“Expression”, as used herein, includes any step involved in the production of a polypeptide (e.g., encoded enzyme) including, but not limited to, transcription, post-transcriptional modification, translation, post-translational modification, and secretion.

“Vector”, as used herein, refers to a nucleic acid molecule capable of transporting another nucleic acid molecule to which it has been linked. One type of vector is a “plasmid”, which refers to a circular double stranded nucleic acid loop into which additional nucleic acid segments can be ligated. Certain vectors are capable of directing the expression of genes to which they are operatively linked. Such vectors are referred to herein as “expression vectors”. Certain other vectors are capable of facilitating the insertion of an exogenous nucleic acid molecule into a chromosome of a host cell, such as a bacterium. Such vectors are referred to herein as “transformation vectors”. In general, vectors of utility in recombinant nucleic acid techniques are often in the form of plasmids. In the present specification, “plasmid” and “vector” can be used interchangeably as the plasmid is the most commonly used form of a vector. Large numbers of suitable vectors are known to those of skill in the art and commercially available.

“Promoter”, as used herein, refers to a sequence of DNA, usually upstream (5′) of the coding region of a structural gene, which controls the expression of the coding region by providing recognition and binding sites for RNA polymerase and other factors which may be required for initiation of transcription. The selection of the promoter will depend upon the nucleic acid sequence of interest. A suitable “promoter” is generally one which is capable of supporting the initiation of transcription in a bacterium of the invention, causing the production of an mRNA molecule.

“Operably linked”, as used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequence. A promoter sequence is “operably-linked” to a gene when it is in sufficient proximity to the transcription start site of a gene to regulate transcription of the gene.

The terms “cassette”, “expression cassette” and “gene expression cassette” refer to a segment of DNA that can be inserted into a target nucleic acid molecule, such as a vector or genomic DNA, at specific restriction sites or by homologous recombination. The segment of DNA comprises a nucleotide sequence that encodes a polypeptide of interest, and the cassette and restriction sites are designed to ensure insertion of the cassette in the proper reading frame for transcription and translation. An expression cassette of the invention may also comprise one or more elements that allow for expression of a nucleotide sequence encoding a polypeptide of interest in a host cell. These elements may include, but are not limited to: a promoter, an enhancer, a response element, a terminator sequence, a polyadenylation sequence, and the like.

“Extrachromosomal”, as used herein, refers to a DNA that is found outside of a chromosome of a cell in question.

“Fusion protein”, as used herein, refers to a protein created through the joining of two or more nucleotide sequences that otherwise would code for separate proteins. This typically occurs through the absence of a stop codon from a DNA sequence coding for the first protein, thereby appending the DNA sequence of the second protein in frame. The DNA sequence will then be expressed by a cell as a single protein with functional properties derived from each of the original proteins. A fusion protein contains all functional domains of the parent proteins.

“% sequence identity” or “% identity”, as used herein, refers to identity between two nucleotide or amino acid sequences. Identity can be determined by comparing a position in each sequence which may be aligned for purposes of comparison. When a position in the compared sequence is occupied by the same base or amino acid, then the molecules are identical at that position. A degree of identity between nucleic acid or amino acid sequences is a function of the number of identical or matching nucleotides or amino acids at positions shared by the nucleotide or amino acid sequences, respectively. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.

More particularly, “% sequence identity” of an amino acid sequence to a reference amino acid sequence, as used herein, defines the % sequence identity calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, percentage identity is calculated by expressing the number of matches as a percentage of the number of amino acids in the reference amino acid sequence.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### Examples

Materials and Methods

Bacterial Strains and Culture Conditions

Amycolatopsis sulphurea NRRL2822 was used for production of CHD and as a source of DNA and for microbiological manipulations. Streptomyces rimosus M4018 was used as a source of DNA (Rhodes et al. 1984). S. albus de114 (Myronovskyi et al., 2018) was used for heterologous expression of CHD biosynthetic gene cluster. Escherichia coli DH10β was used for standard cloning procedures (Sambrook, Russell 2001), E. coli ET12567 (MacNeil et al. 1992) and SCS110 (Stratagene) strains for isolation of non-methylated plasmid DNA, suitable for transformation of A. sulphurea, and E. coli GB2006 (Gene Bridges) for preparation of A. sulphurea cosmid library. Escherichia coli ET12567 carrying PUZ8002 plasmid (Paget et al. 1999) was used as a donor strain for intergeneric conjugation with S. albus de114. Soya mannitol (MS) agar and tryptone soy broth (TSB) (Kieser et al. 2000) with incubation at 30° C. were used for sporulation and cultivation of actinomycetes in liquid medium, respectively. For CHD and CHD analogues production, A. sulphurea was cultivated in CH-V seed medium (1.5% soy flour, 0.1% yeast extract, 1.5% glucose, 0.5% NaCl, 0.1% CaCO₃, pH 7.0) and CH—F2 production medium (2% soy flour, 0.5% yeast extract, 0.2% CaCO₃, 0.05% citric acid, 5% glucose, pH 7.0) (adapted from (Oliver et al. 1962; Oliver, Sinclair) and (Mitscher et al. 1983)). Cultivations were performed in Falcon tubes at 30° C. on a rotary shaker at 220 rpm for 36 h in seed medium with 15% (v/v) used to inoculate CH—F2 production medium and cultivated for further 7 days under the same conditions. For heterologous production of CHD and CHD analogues, S. albus was cultivated in TSB seed medium and four different production media: CH—F2, DNPM (4% dextrin, 0.75% soytone, 0.5% baking yeasts, 2.1% MOPS, pH 6.8 (Bilyk et al. 2016)), NLSY (0.1% NaCl, 0.1% KH₂PO₄, 0.05% MgSO₄×7H₂O, 2.5% glycerol, 0.584% L-glutamin, 0.2% trace elements solution, 1% yeast extract, pH 7.3 (Bilyk et al. 2017), and SG1 (2% glucose, 0.5% yeast extract, 1% soytone, 0.2% CaCO₃, pH 7.2 (Koshla et al. 2017)). Cultivations were performed in Falcon tubes at 30° C. on a rotary shaker at 220 rpm for 36 h in seed medium with 5% (v/v) used to inoculate production media and cultivated for further 7 days under the same conditions. For transformation of A. sulphurea, S27M and R2L media were used (Madon, Hutter 1991). Apramycin (Apr; 200 μg mL⁻¹), erythromycin (Erm; 20 μg mL⁻¹) or kanamycin (Kan; 300 μg mL⁻¹) was used for selection of A. sulphurea transformants on S27M. For further subcultivation of A. sulphurea transformants, MS was supplemented with Apr (400 μg mL⁻¹), Erm (20 μg mL⁻¹) or kanamycin (400 μg mL⁻¹). For intergeneric conjugation between S. albus and E. coli MS medium, supplemented with 10 mM MgCl₂ was used. Apramycin (50 μg mL⁻¹) together with nalidixic acid (25 μg mL⁻¹) was used for selection of S. albus exconjugants on MS. For selection of E. coli transformants, ampicillin (Amp; 100 μg mL⁻¹), Apr (50 μg mL⁻¹), Kan (25 μg mL⁻¹) or chloramphenicol (Cm; 10 μg mL⁻¹) were added into LB medium.

DNA Isolation and Manipulation

Isolation and manipulation of DNA in E. coli (Table 2) were carried out according to standard protocols (Sambrook, Russell 2001; Kieser et al. 2000). Transformation of A. sulphurea NRRL2822 was carried out by the protocol for transformation of A. mediterranei (Madon, Hutter 1991), using vectors pAB03 and pNV18 already described previously (Lukezic et al. 2013). Cosmids were introduced into S. albus de114 via conjugation (Kieser et al. 2000).

Sequencing of Genomic DNA

Salting out procedure (Kieser et al. 2000) was used to isolate genomic DNA from A. sulphurea which was sequenced by Illumina sequencing.

Preparation of A. sulphurea Cosmid Library

Genomic DNA was partially digested with Sau3AI and the DNA fragments of approximate size 35-40 kb were ligated into the BamHI site of replicative conjugative cosmid vector pOJ456, a modified version of the pOJ436 vector (Bierman et al. 1992), where 2.5 kb ΦC31 integrase cassette was excised with HindIII (overhangs were filled in with Klenow polymerase) and replaced with 2.5 kb pSG5 replication cassette excised with Eco81I and SphI (overhangs were filled in with Klenow polymerase) from medium copy number vector pKC1139 (Bierman et al. 1992). The ligated DNA was packaged into phage particles (Gigapack III Gold Packaging kit, Agilent Technologies) and introduced into E. coli GB2006.

Identification and Sequencing of Cosmid Carrying CHD Biosynthetic Gene Cluster

The cosmid library was screened by combining all 3400 colonies and streaking the mixture onto LB agar plates supplemented with 3 μg mL⁻¹ of CHD to select for CHD-resistant single colonies expressing ChdR efflux pump encoded in the CHD biosynthetic gene cluster. 18 positive clones were selected to isolate cosmid DNA and additional PCR screening was carried out using the primer pairs CobU1/CobU2 and glu1/glu2 (Table 3), designed to anneal to the flanking regions of CHD biosynthetic gene cluster. Based on the PCR screen, two cosmids were selected for complete sequencing by Illumina sequencing, resulting in confirmation of cosmid pOJ456CHD12, carrying the complete CHD biosynthetic gene cluster, whose correct and complete sequence was also identified from genomic DNA sequence of A. sulphurea.

Variations of Cosmids Carrying CHD Biosynthetic Gene Cluster

34 kbp CHD biosynthetic gene cluster from pOJ456CHD12 was cloned via SpeI and XbaI into integrative conjugative cosmids pOJ436, pOJ436e*chdR, pOJ436e*oxyDP, or pOJ436e*oxyDPchdR, resulting in pOJ436CHD12, pOJ436e*chdRCHD12, pOJ436e*oxyDPCHD12, and pOJ436e*oxyDPchdRCHD12, respectively. pOJ436e*chdR, pOJ436e*oxyDP, or pOJ436e*oxyDPchdR were constructed from pOJ436 by introducing 1.8 kb, 3.2 kb and 4.7 kb fragments, carrying chdR, oxyDP and oxyDPchdR genes, respectively, all under the control of P_{ermE*} promoter. Fragments were excised with Ecl136II (overhang was filled in with Klenow polymerase) and XbaI from plasmids pAB03e*chdR, pAB03e*oxyDP and pAB03e*oxyDPchdR, respectively, and used to replace the 1.9 kb fragment in pOJ436, excised with NruI (overhang was filled in with Klenow polymerase) and XbaI. pAB03e*chdR was constructed by cloning 1.5 kb chdR gene, amplified by PCR using primers chdRF and chdRR, digested with NdeI and XbaI and ligated into pAB03e* (pAB03 vector with P_{ermE*} promoter instead of actII-ORF4/P_{actl} activator/promoter system). pAB03e*oxyD was constructed by cloning oxyD gene, excised from pAB03oxyD (Lesnik et al., 2015) with NdeI and XbaI and ligated into pAB03e*. pAB03e*oxyDP was constructed by cloning oxyP gene, excised from pAB03oxyDP (Lesnik et al., 2015) with XbaI, into XbaI site of pAB03e*oxyD downstream of oxyD gene. pAB03e*oxyDPchdR was constructed by cloning chdR gene, excised from pAB03e*chdR with C/al and HindIII (overhangs were filled in with Klenow polymerase), into XbaI (overhangs were filled in with Klenow polymerase) site of pAB03e*oxyDP downstream of oxyP gene.

Heterologous Expression of CHD Biosynthetic Gene Cluster

Cosmids carrying the CHD biosynthetic gene cluster, pOJ456CHD12, pOJ436CHD12, pOJ436e*chdRCHD12, pOJ436e*oxyDPCHD12 and pOJ436e*oxyDPchdRCHD12, and empty control cosmids, pOJ456, pOJ436, pOJ436e*chdR, pOJ436e*oxyDP and pOJ436e*oxyDPchdR, were transformed into E. coli ET12567 (MacNeil et al. 1992) carrying PUZ8002 which was then used as donor strain for intergeneric conjugation with S. albus de114. MS plates supplemented with 10 mM MgCl₂ were overlaid with Apr and nalidixic acid after overnight incubation. Each exconjugant was further repatched onto MS agar containing Apr (50 μg mL⁻¹) and nalidixic acid (25 μg mL⁻¹), followed by inoculation into TSB medium as seed culture for production media CH—F2 (2% soy flour, 0.5% yeast extract, 0.2% CaCO₃, 0.05% citric acid, 5% glucose, pH 7.0) (adapted from (Oliver et al. 1962; Oliver, Sinclair) and (Mitscher et al. 1983)), DNPM (4% dextrin, 0.75% soytone, 0.5% baking yeasts, 2.1% MOPS, pH 6.8 (Bilyk et al. 2016)), NLSY (0.1% NaCl, 0.1% KH₂PO₄, 0.05% MgSO₄×7H₂O, 2.5% glycerol, 0.584% L-glutamin, 0.2% trace elements solution, 1% yeast extract, pH 7.3 (Bilyk et al. 2017)), and SG1 (2% glucose, 0.5% yeast extract, 1% soytone, 0.2% CaCO₃, pH 7.2 (Koshla et al. 2017)). Cultivations were performed in Falcon tubes at 30° C. on a rotary shaker at 220 rpm for 36 h in seed medium with 5% (v/v) used to inoculate production media and cultivated for further 7 days under the same conditions. Culture broths were extracted and analysed by LC-MS to check for production of CHD or CHD analogues.

Site-Directed Mutagenesis of chdY in A. sulphurea

The mutation was introduced using the double cross-over approach to replace the target gene with the mutated gene. Catalytic residue Glyl76 of ChdY was replaced by Ser. First, vector for homologous recombination was constructed: ermE gene was amplified by PCR using primers FSB01C and FSB02C (Table 3) and later digested with EcoRI and XbaI to obtain the 1.6 kb fragment, which was ligated into pNV18 to obtain pNV18Erm. To mutate residue Glyl76 via site-directed mutagenesis, 0.7 kb upstream and 0.7 kb downstream fragments were amplified, by using primer pairs chdYserLF/chdYserLR and chdYserRF/chdYserRR. Primers labeled with LR (left reverse) and RF (right forward) were designed to anneal to the region containing the catalytic residue Glyl76 and introduce the desired mutation (Table 3). Third PCR was performed with outer set of primers, chdYserLF and chdYserRR, using previous two PCR products as template, which were overlapping in the region where the mutation was introduced, yielding 1.4 kb fragment. Resulting fragment was digested with SphI and SpeI and ligated into pNV18Erm to obtain pNV18ErmchdYser, which was transformed into E. coli SCS110 (Stratagene) to obtain the non-methylated plasmid, which was then introduced into A. sulphurea via direct transformation of mycelium (Madon, Hutter 1991). S27M plates were overlaid with Erm after overnight incubation. Each transformant colony was further re-patched onto MS agar containing Erm and subcultivated. After three or more subcultivations in TSB without antibiotic, Erm-sensitive (Erms) colonies (secondary recombinants) were isolated. To confirm that secondary recombinants contain the introduced mutation and are not revertants to wild-type, colony PCR using the outer pair of primers labeled with LF (left forward) and RR (right reverse), chdYserLF and chdYserRR, respectively, followed by DNA sequencing, was performed.

Homologous Expression of Wild-Type chdY and chdOII Genes in Mutant Strain of A. sulphurea

A. sulphurea mutant obtained through previously described site-directed mutagenesis approach was complemented with wild-type genes chdY and chdOII-chdY from A. sulphurea. Genes for ChdY (cyclase) and ChdOII-ChdY (oxygenase-cyclase fusion) were amplified by PCR using chdYF/chdYR or chdOIIF/chdYR sets of primers, respectively, and genomic DNA of A. sulphurea as a template. PCR products were digested with NdeI and XbaI and separately cloned into pAB03, resulting in pAB03chdY and pAB03chdOII-chdY (Table 2), respectively. Constructs were confirmed by sequencing, transformed into E. coli SCS110 and introduced into A. sulphurea mutant via direct transformation of mycelium (Madon, Hutter 1991). Plasmids pAB03chdY and pAB03chdOII-chdY were separately integrated into A. sulphurea ChdY-G176S.

Heterologous Expression of oxyN in Mutant Strain of A. sulphurea

A. sulphurea mutant was complemented also with heterologous gene oxyN. OxyN (cyclase) (Zhang et al. 2006) from S. rimosus M4018 (Rhodes et al. 1984) was amplified by PCR using primers oxyN Fw and oxyN Rv (Table 3), digested with NdeI and XbaI and cloned into pAB03 vector, resulting in pAB03oxyN(Table 2). Construct was confirmed by sequencing, transformed into E. coli SCS110 and introduced into mutated strain of A. sulphurea via direct transformation of mycelium (Madon, Hutter 1991). Plasmid pAB03oxyN was integrated into A. sulphurea ChdY-G176S.

TABLE 2
Bacterial strains and plasmids used herein^{[a]}

| | | |
|---|---|---|
| | | Reference |
| Strain or plasmid | Relevant characteristics | or source |

Escherichia coli

| | | |
|---|---|---|
| DH10β | F- endA1 recA1 galE15 galK16 upG rpsL ΔlacX74 | Invitrogen |
| | Φ80/acZΔM15 araD139 Δ(ara-leu)7697 mcrA Δ(mrr- | |
| | hsdRMS-mcrBC) λ- | |
| ET12567 | F- dam13::Tn9, dcm6, hsdM, hsdR, recF143::Tn1I, | (MacNeil et |
| | galK2, galT22, ara14, lacY1, xyl5, leuB6, thi1, | al. 1992) |
| | tonA31, rpsL136, hisG4, tsx78, mtl1 glnV44 | |
| SCS110 | rpsL (Str^{r}) thr leu endA thi-1 lacY galK galT ara tonA | Stratagene |
| | tsx dam dcm supE44 Δ(lac-proAB) [F′ traD36 proAB | |
| | lacl^{q}ZΔM15] | |
| GB2006 | δM109 rpsL- ΔafuA | Gene |
| | | Bridges |

Amycolatopsis sulphurea

| | | |
|---|---|---|
| NRRL 2822 | Wild-type producer of chelocardin | ARS |
| | | Culture |
| | | Collection |

Streptomyces rimosus

| | | |
|---|---|---|
| M4018 | Producer of oxytetracycline | (Rhodes et |
| | | al. 1984) |

Streptomyces albus

| | | |
|---|---|---|
| S. albus del14 | Host strain for heterologous expression | (Myronovskyi |
| | | et al. 2018) |

Plasmids

| | | |
|---|---|---|
| pNV18 | Kan^{r}, lacZα | (Chiba et al. |
| | | 2007) |
| pNV18Erm | Kan^{r}, Erm^{r}, lacZα | This study |
| pAB03 | pSET152-derived, containing ΦBT, Apr^{r} | (Lukezic et |
| | | al. 2013) |
| pAB03oxyD | oxyD cloned into pAB03 | (Lesnik et al. 2015) |
| pAB03oxyDP | oxyD and oxyP cloned into pAB03 | (Lesnik et |
| | | al. 2015) |
| pOJ436 | pSET152-derived cosmid, containing ΦC31, Apr^{r} | (Bierman et |
| | | al. 1992) |
| pOJ436CHD12 | pOJ436 cosmid carrying CHD biosynthetic cluster | This study |
| pKC1139 | bifunctional oriT RK2 vector, pSG5 ori, Apr^{r} | (Bierman et |
| | | al. 1992) |
| pOJ456 | pOJ436-derived cosmid, ΦC31 integrase cassette | This study |
| | replaced with pSG5 replication cassette, Apr^{r} | |
| pOJ456CHD12 | pOJ456 cosmid carrying CHD biosynthetic cluster | This study |
| pAB03e* | pAB03 vector with P_{ermE*} | Acies Bio |
| | promoter instead of actII- | d.o.o. |
| | ORF4/Pactl activator/promoter system | |
| pAB03e*chdR | pAB03e* carrying chdR gene | This study |
| pAB03e*oxyD | pAB03e* carrying oxyD gene | This study |
| pAB03e*oxyDP | pAB03e* carrying oxyD and oxyP genes | This study |
| pAB03e*oxyDPchdR | pAB03e* carrying oxyD, oxyP and chdR genes | This study |
| pOJ436e*chdR | pOJ436 carrying a 1, 8 kb fragment from | This study |
| | pAB03e*chdR containing chdR gene under the | |
| | control of P_{ermE*} promoter | |
| pOJ436e*oxyDP | pOJ436 carrying a 3, 2 kb fragment from | This study |
| | pAB03e*oxyDP containing oxyD and oxyP genes | |
| | under the control of P_{ermE*} promoter | |
| pOJ436e*oxyDPchdR | pOJ436 carrying a 4, 7 kb fragment from | This study |
| | pAB03e*oxyDPchdR containing oxyD, oxyP and | |
| | chdR genes under the control of P_{ermE*} | |
| | promoter | |
| pOJ436e*chdRCHD12 | pOJ436e*chdR carrying also CHD biosynthetic | This study |
| | cluster | |
| pOJ436e*oxyDPCHD12 | pOJ436e*oxyDP carrying also CHD biosynthetic | This study |
| | cluster | |
| pOJ436e*oxyDPchdRCH | pOJ436e*oxyDPchdR carrying also CHD biosynthetic | This study |
| D12 | cluster | |
| pNV18ErmchdYser | Fragment containing cyclase gene chdY with | This study |
| | mutation Gly176Ser cloned into pNV18Erm | |
| pAB03chdY | Cyclase gene chdY cloned into pAB03 | This study |
| pAB03chdOII-chdY | Gene chdOII-chdY with oxygenase and cyclase | This study |
| | domain cloned into pAB03 | |
| pAB03oxyN | Cyclase gene oxyN cloned into pAB03 | This study |

^{[a]}Apr^{r}, apramycin resistant; Erm^{r}, erythromycin resistant; Kan^{r}, kanamycin resistant

TABLE 3
Sequences of oligonucleotide primers for
PCR experiments used in this study^{[a]}

| Primers | Sequence |
|---|---|
| CobU1 | 5′-TCCTCACTGCAGGTCGAGTACC-3′ |
| CobU2 | 5′-CGGGAAGTCGCGGTATGC-3′ |
| glu1 | 5′-CGCGCTGGTCAAAGTCTACG -3′ |
| glu2 | 5′-CTGGACGCCTCGCCGTAC-3′ |
| chdRF | 5′-TATATACATATGAAGGACAATCTCGCGAGA-3′ |
| chdRR | 5′-TATATATCTAGAGGACCTCCGCATCAGGC-3′ |
| FSB01C | 5′-AGTCGAATTCGCACCATATGAGACCAAGCGCG |
| | TCCGGGTG-3′ |
| FSB02C | 5′-CGACTCTAGAGGATCACTAGTTACCAGCCCGA |
| | CCCGAGCACGC-3′ |
| chdYserLF | 5′-TATATAGCATGCGCATCATCGACC-3′ |
| chdYserLR | 5′-GCGTCGGTGCTGATGACCC-3′ |
| chdYserRF | 5′-GGTCATCAGCACCGACGCG-3′ |
| chdYserRR | 5′-TATATAACTAGTCGTCCAGCTGCAGCAGATAAC-3′ |
| chdYF | 5′-ATATACATATGCGCATCATCGACCTGTC-3′ |
| chdYR | 5′-TATATATCTAGACTAGTCCAGCAGGGCAACGG-3′ |
| chdOIIF | 5′-ATATACATATGCCTGAGGACTCCGGC-3′ |
| oxyNFw | 5′-TATATACATATGCGCATCATCGATCTGTCGA-3′ |
| oxyNRv | 5′-ATATATCTAGACTACTCCTCCACCACCGCC-3′ |

^{[a]}Restriction sites are underlined, introduced point-mutations are in bold

LC-MS Analysis

To check for production of CHD and CHD analogues, A. sulphurea or S. albus culture broths were acidified to pH 1-2 with 50% TFA, followed by extraction with 2V of MeOH. The extract was centrifuged and analyzed by LC-MS. All measurements were performed on a Dionex Ultimate 3000 LC system using a Luna C-18 (2) HST, 100×2.0 mm, 2.5 μm column (Phenomenex). Separation of 1 μl sample was achieved by a linear gradient from (A) H₂O+0.1% FA to (B) ACN+0.1% FA at a flow rate of 500 μl/min and 45° C. The gradient was initiated by a 0.5 min isocratic step at 5% B, followed by an increase to 95% B in 9 min to end up with a 1.5 min step at 95% B before reequilibration with initial conditions. UV spectra were recorded by a DAD in the range from 200 to 600 nm. The MS measurement was carried on an amaZon speed mass spectrometer (BrukerDaltonics, Bremen, Germany) using the standard ESI source. Mass spectra were acquired in centroid mode ranging from 200-2000 m/z in positive ionization mode.

Results and Discussion

CHD Biosynthetic Gene Cluster

After sequencing the genomic DNA of CHD producer, A. sulphurea, one additional gene in the CHD biosynthetic gene cluster, essential for the biosynthesis of CHD, and two more regulatory genes were discovered lying downstream of already identified CHD biosynthetic genes. The newly discovered biosynthetic gene is chdY, encoding a putative second ring cyclase, homologous to OxyN from oxytetracycline biosynthesis (Pickens, Tang 2010). Interestingly, ChdOII and ChdY are encoded as fusion proteins (opposite to separately encoded homologs found in OTC biosynthetic gene cluster, OxyL and OxyN) and similar is observed for chdL and chdOIII nucleotide sequences which are also operably linked to form a fusion protein of the respective polypeptides encoded by them. The same is true for homologs from OTC biosynthetic gene cluster, oxyH and oxyG, respectively. Bioinformatic analysis of the sequence downstream of biosynthetic genes revealed two regulatory genes, encoding SARP and LuxR, which are also found to regulate OTC and CTC biosynthesis (Lesnik et al. 2009; Yin et al. 2015).

CHD Biosynthesis

Biosynthesis of CHD can be directly compared to OTC, as all oxy genes (Pickens, Tang 2010) responsible for the generation of basic TC scaffold have homologs in CHD biosynthetic gene cluster and also one of the intermediates in OTC biosynthesis, 4-keto-ATC strongly resembles putative CHD precursor, 4-keto-9-desmethyl-CHD, differing only in the moiety at C2 position, resulting from incorporation of a different starter unit. However, intermediate in OTC biosynthesis, leading to an impurity, ADOTC, which is primed by acetate (as CHD), should then be the same as in CHD biosynthesis, 4-keto-9-desmethyl-CHD.

Polyketide skeleton of CHD is supposedly synthesized, as previously described (Lukezic et al. 2013), by type II minimal polyketide synthase (minimal PKS) genes, consisting of ketosynthase α, ketosynthase β and acyl carrier protein (ACP), designated as ChdP, ChdK and ChdS, respectively (FIGS. 1 and 2), condensating 10 malonate-derived building blocks into acetate-primed decaketide. The malonyl-CoA:ACP acyltransferase, needed for the transfer of the extender unit malonyl-CoA to ACP, was proposed to be shared with fatty acid biosynthesis (Revill et al. 1995). As in OTC biosynthesis by OxyJ (Pickens, Tang 2010), initial folding of the growing polyketide chain is most probably directed by a ketoreductase ChdT, reducing the keto group at C9 (FIG. 2). Closure of rings leading to the formation of CHD backbone is most likely directed by aromatases/cyclases ChdQI, ChdQII, ChdY and ChdX, first two being similar to OxyK and the last two homologous to OxyN and OxyI, respectively (Pickens, Tang 2010). Based on homologies to aromatases, encoded in other aromatic polyketide biosynthetic gene clusters, we believe that didomain aromatases ChdQI and ChdQII are responsible for first ring (D) formation (4 in FIG. 2), while monodomain cyclase ChdY is needed for second ring (C) closure. As in biosynthesis of other aromatic polyketides, formation of third ring (B) could be spontaneous (5 in FIG. 2). Candidate for the last ring (A) cyclization is cyclase ChdX, deducing from comparison with chromomycin and mithramycin biosynthesis (Menendez et al. 2004), while the function of its homologue in OTC biosynthesis, OxyI, on the other hand, has not been elucidated yet (Pickens, Tang 2010). Such generated tetracyclic scaffold is then further processed towards CHD through different post-PKS tailoring reactions, the last two also leading CHD biosynthesis away from that of typical tetracyclines. ChdMI, OxyF homologue (Zhang et al. 2007), could methylate C6 position in CHD biosynthetic intermediate, while oxygenase pair ChdOII and ChdOI, homologs of OxyL and OxyE (Wang et al. 2009), respectively, could be responsible for a double hydroxylation of ring A at C4/C12a. Hydroxylation at C4 is a followed by transamination by ChdN, a PLP-dependent aminotransferase only distantly related to OxyQ, which is responsible for incorporation of an amino group at C4 in OTC biosynthesis (Pickens, Tang 2010). The activity of such different aminotransferases represents a diverging point between CHD and typical TCs biosynthesis and results in different products: amino group incorporated into CHD is in R-configuration, while the one in OTC biosynthesis stands in S-confguration. In contrast to more decorated backbone of typical TCs, there is only one more tailoring reaction leading to CHD, C9-methylation, which is believed to be catalysed by ChdMII, homolog of C9-methyltransferases from chromomycin and mithramycin biosynthesis (Menendez et al. 2004).

Regulation of CHD Biosynthesis and Self-Resistance

One of the putative regulatory proteins found in CHD biosynthetic cluster belongs to the Streptomyces antibiotic regulatory protein (SARP) transcription activators. It is homologous to OtcR, identified by Yin et al. (Yin et al. 2015), which acts as a positive pathway-specific activator of OTC biosynthesis leading to a significant increase in OTC production when overexpressed at the appropriate level. The second putative regulatory protein, found in CHD biosynthetic gene cluster, belongs to the LuxR family and is homologous to regulatory protein OtcG from OTC biosynthesis, identified by Lešnik et al. (Lesnik et al. 2009). OtcG has a conditionally positive role in OTC biosynthesis: its inactivation reduced the production of OTC by more than 40%, while its overexpression under the strong constitutive promoter P_{ermE*} did not yield any statistically significant change in the production of OTC (Lesnik et al. 2009). chdR encodes a putative integral membrane protein that is most probably responsible for the efflux of CHD from the cell and is probably regulated by another regulatory protein, the putative TetR family repressor protein ChdA (Lukezic et al. 2013).

Heterologous Expression of CHD Biosynthetic Gene Cluster

Replicative cosmid pOJ456-CHD12, carrying CHD biosynthetic gene cluster, was fished out from A. sulphurea cosmid library by selection on CHD containing agar plates. After confirming its correct sequence, the cosmid was introduced into S. albus by conjugation in attempt to heterologously express CHD biosynthetic gene cluster. The CHD biosynthetic cluster was transferred into an integrative cosmid (pOJ436) to allow a stable integration of CHD biosynthetic cluster into the genome of heterologous host. Indeed, heterologous expression of CHD biosynthetic cluster from integrated cosmid pOJ436-CHD12 was successful and resulted in production of CHD, reaching up to approx. 50 mg/L.

Furthermore, we constructed another integrative cosmid carrying CHD biosynthetic cluster with additional copy of CHD efflux pump gene chdR under strong promoter P_{erm*} (construct pOJ436-PermE*-chdR-CHD12) to overcome possible self-resistance issues during heterologous expression of CHD. Additional copy of efflux pump gene chdR led to slightly increased production yields of CHD up to approx. 60 mg/L (FIG. 3).

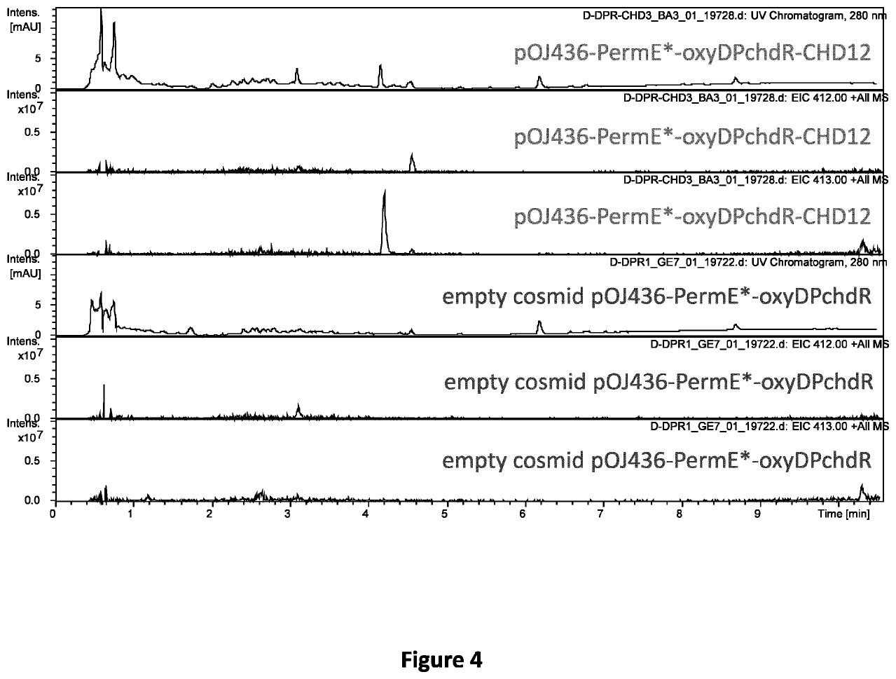

Additionally, with the aim to produce CDCHD (Lesnik et al. 2015), we constructed integrative cosmid carrying the CHD biosynthetic cluster and oxyDPchdR genes under strong promoter P_{erm*} (construct pOJ436-PermE*-oxyDPchdR-CHD12), whose expression resulted in production of CDCHD (less than 5 mg/L; FIG. 4).

Inactivation and Complementation Experiments

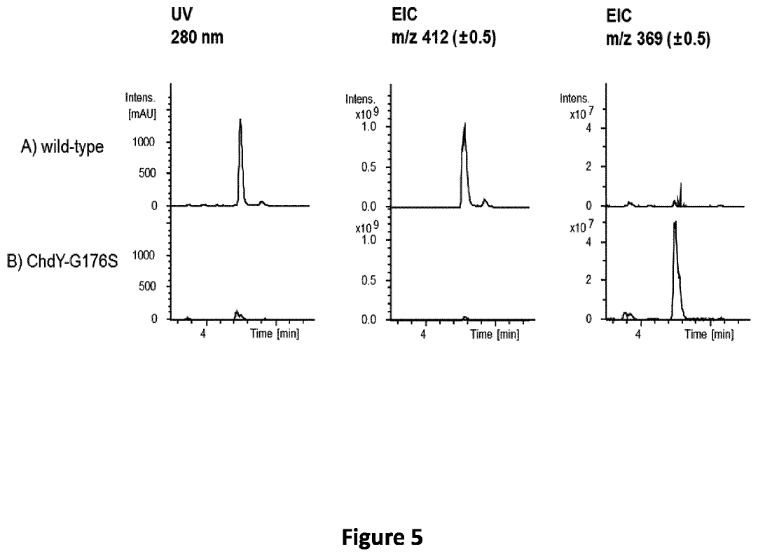

We mutated putative second ring (C) cyclase ChdY residue G176, which was chosen based on comparison with DpsY, a cyclase from daunomycin biosynthesis (Hautala et al. 2003). Mutation of conserved Gly to Ser (G191S), even though most probably not being a part of active site (Diaz-Sàez et al. 2014), resulted in inactivation of DpsY (Hautala et al. 2003). Also in our ChdY inactivation experiment production of CHD was not observed anymore or only in traces (FIG. 5).

Mutation of chdY led to such increase in production of a shunt product (»compound 369«) (FIG. 5), which allowed its isolation and structure elucidation by HRMS and NMR analysis. The HRMS mass for “compound 369” was found to be 369.09 [M⁺+H], which corresponds to the expected mass of 369.0969 for C₂₀H₁₇O₇⁺. This shunt product is most probably the result of spontaneous cyclization following first ring (D) closure and aromatization mediated by intact ketoreductase ChdT and aromatase pair ChdQI/ChQII.

The mutant was then complemented with non-mutated wild-type genes chdY or chdOII-chdY (encoding a fusion protein as found in CHD cluster), which partly restored the production of CHD. Such complemented mutants were necessary as they represent, contrary to wild-type CHD producer strain, directly comparable controls for later complementation experiment with homologous enzyme from OTC biosynthesis. In all complementation experiments the genes were introduced by integration of pAB03 plasmid, carrying the selected genes, into a genome location distant from the wild-type location in CHD biosynthetic gene cluster, allowing the mutant to produce both, the mutated and wild-type protein. For complementation experiment we chose a homologous enzyme from OTC biosynthesis, whose function was demonstrated by heterologous expression in Streptomyces host and isolation of shunt products.

6% restored production of CHD after complementation of chdY mutant with OxyN (second ring cyclase in OTC biosynthesis) compared to 3% restored CHD production with wild-type ChdY or similarly 5% restored CHD production with whole fusion protein ChdOII-ChdY (FIG. 6), led us to the conclusion that ChdY is responsible for second (C) ring cyclization. CHD production in negative control with integrated empty plasmid was less than 0.3% of production level in wild-type strain.

In the inactivation mutant traces of CHD production was still observed, which could possibly be due to some remaining catalytic activity of mutated cyclase or spontaneous cyclization leading to synthesis of small amounts of CHD. The reason for low production of CHD after complementation could be because complemented mutant generated through site-directed mutagenesis is still expressing both, the mutated and introduced wild-type protein, which are both taking part in PKS complex structure. Incorporation of structurally similar but functionally impaired mutated protein might thus prevent the PKS complex to reach its full biosynthetic potential.

### LIST OF CERTAIN REFERENCES CITED IN THE DESCRIPTION

Abelson, John N.; Simon, Melvin I. (1998): Methods in enzymology. Cumulative subject index. Vols. 263, 264, 266-289/editors-in-chief, John N. Abelson and Melvin I. Simon. London: Academic Press. Available online at http://www.elsevier.com/journals BLDSS.
Ames, B. D.; Korman, T. P.; Zhang, W.; Smith, P.; Vu, T.; Tang, Y.; Tsai, S. C. (2008): Crystal structure and functional analysis of tetracenomycin ARO/CYC. implications for cyclization specificity of aromatic polyketides. In Proc Natl Acad Sci USA 105 (14), pp. 5349-5354. DOI: 10.1073/pnas.07092231050709223105.
Ausubel, Frederick M. (1987-): Current protocols in molecular biology. Brooklyn, N. Y.: Greene Publishing Associates; Media. Available online at http://onlinelibrary.wiley.com/BLDSS.
Bierman, M.; Logan, R.; O'Brien, K.; Seno, E. T.; Rao, R. N.; Schoner, B. E. (1992): Plasmid cloning vectors for the conjugal transfer of DNA from Escherichia coli to Streptomyces spp. In Gene 116 (1), pp. 43-49.
Bilyk, Bohdan; Horbal, Liliya; Luzhetskyy, Andriy (2017): Chromosomal position effect influences the heterologous expression of genes and biosynthetic gene clusters in Streptomyces albus J1074. In Microb Cell Fact 16 (1), p. 5. DOI: 10.1186/s12934-016-0619-z.
Bilyk, Oksana; Sekurova, Olga N.; Zotchev, Sergey B.; Luzhetskyy, Andriy (2016): Cloning and Heterologous Expression of the Grecocycline Biosynthetic Gene Cluster. In PloS one 11 (7), e0158682. DOI: 10.1371/journal.pone.0158682.
Chiba, K.; Hoshino, Y.; Ishino, K.; Kogure, T.; Mikami, Y.; Uehara, Y.; Ishikawa, J. (2007): Construction of a pair of practical Nocardia-Escherichia coli shuttle vectors. In Jpn. J. Infect. Dis. 60 (1), pp. 45-47.
Diaz-Sàez, Laura; Srikannathasan, Velupillai; Zoltner, Martin; Hunter, William N. (2014): Structures of bacterial kynurenine formamidase reveal a crowded binuclear zinc catalytic site primed to generate a potent nucleophile. In Biochem J 462 (3), pp. 581-589. DOI: 10.1042/BJ20140511.
Fernandez-Moreno, M. A.; Martinez, E.; Boto, L.; Hopwood, D. A.; Malpartida, F. (1992): Nucleotide sequence and deduced functions of a set of cotranscribed genes of Streptomyces coelicolor A3(2) including the polyketide synthase for the antibiotic actinorhodin. In J. Biol. Chem. 267 (27), pp. 19278-19290.
Harnes, B. D.; Higgins, S. J. (1984): Transcription and translation. A practical approach/edited by B. D. Hames, S. J. Higgins. Oxford: IRL (Practical approach series).
Hautala, Anne; Torkkell, Sirke; Raty, Kaj; Kunnari, Tero; Kantola, Jaana; Mantsala, Pekka et al. (2003): Studies on a second and third ring cyclization in anthracycline biosynthesis. In J. Antibiot. 56 (2), pp. 143-153.
Hopwood, D. A.; Sherman, D. H. (1990): Molecular genetics of polyketides and its comparison to fatty acid biosynthesis. In Annu Rev Genet 24, pp. 37-66. DOI: 10.1146/annurev.ge.24.120190.000345.
Kieser, T.; Bibb, M. J.; Buttner, M. J.; Chater, K. F.; Hopwood, D. A. (2000): Practical Streptomyces Genetics. Norwich: John Innes Foundation.
Koshla, Oksana; Lopatniuk, Maria; Rokytskyy, Ihor; Yushchuk, Oleksandr; Dacyuk, Yuriy; Fedorenko, Victor et al. (2017): Properties of Streptomyces albus J1074 mutant deficient in tRNALeuUAA gene bldA. In Arch Microbiol 199 (8), pp. 1175-1183. DOI: 10.1007/s00203-017-1389-7.
Lesnik, U.; Gormand, A.; Magdevska, V.; Fujs, S.; Raspor, P.; Hunter, I.; Petkovic, H. (2009): Regulatory elements in tetracycline-encoding gene clusters. the otcG gene positively regulates the production of oxytetracycline in Streptomyces rimosus. In Food Technology & Biotechnology 47 (3), pp. 323-330.
Lesnik, Urska; Lukezic, Tadeja; Podgorsek, Ajda; Horvat, Jaka; Polak, Tomaz; Sala, Martin et al. (2015): Construction of a new class of tetracycline lead structures with potent antibacterial activity through biosynthetic engineering. In Angew. Chem., Int. Ed. 54 (13), pp. 3937-3940. DOI: 10.1002/anie.201411028.
Lukezic, T.; Lesnik, U.; Podgorsek, A.; Horvat, J.; Polak, T.; Sala, M. et al. (2013): Identification of the chelocardin biosynthetic gene cluster from Amycolatopsis sulphurea. a platform for producing novel tetracycline antibiotics. In Microbiology 159 (Pt 12), pp. 2524-2532. DOI: 10.1099/mic.0.070995-0mic.0.070995-0.
MacNeil, D. J.; Gewain, K. M.; Ruby, C. L.; Dezeny, G.; Gibbons, P. H.; MacNeil, T. (1992): Analysis of Streptomyces avermitilis genes required for avermectin biosynthesis utilizing a novel integration vector. In Gene 111 (1), pp. 61-68.
Madon, J.; Hutter, R. (1991): Transformation system for Amycolatopsis (Nocardia) mediterranei. direct transformation of mycelium with plasmid DNA. In J. Bacteriol. 173 (20), pp. 6325-6331.
Martin, J. L.; McMillan, F. M. (2002): SAM (dependent) I AM. the S-adenosylmethionine-dependent methyltransferase fold. In Curr Opin Struct Biol 12 (6), pp. 783-793.
Mason, J. R.; Cammack, R. (1992): The electron-transport proteins of hydroxylating bacterial dioxygenases. In Annu Rev Microbiol 46, pp. 277-305. DOI: 10.1146/annurev.mi.46.100192.001425.
Menendez, N.; Nur-e-Alam, M.; Brana, A. F.; Rohr, J.; Sales, J. A.; Mendez, C. (2004): Biosynthesis of the antitumor chromomycin A3 in Streptomyces griseus. analysis of the gene cluster and rational design of novel chromomycin analogs. In Chem Biol 11 (1), pp. 21-32. DOI: 10.1016/j.chembiol.2003.12.011S1074552103002837.
Mitscher, L. A.; Swayze, J. K.; Hogberg, T.; Khanna, I.; Rao, G. S.; Theriault, R. J. et al. (1983): Biosynthesis of cetocycline. In J. Antibiot. 36 (10), pp. 1405-1407.
Molnar, V.; Matkovic, Z.; Tambic, T.; Kozma, C. (1977): Klinicko-farmakolosko ispitivanje kelokardina u bolesnika s infekcijom mokracnih puta. In Lij. vjes. 99, pp. 560-562.
Myronovskyi, Maksym; Rosenkranzer, Birgit; Nadmid, Suvd; Pujic, Petar; Normand, Philippe; Luzhetskyy, Andriy (2018): Generation of a cluster-free Streptomyces albus chassis strains for improved heterologous expression of secondary metabolite clusters. In Metab Eng. DOI: 10.1016/j.ymben.2018.09.004.
Oliver, T. J.; Prokop, J. F.; Bower, R. R.; Otto, R. H. (1962): Chelocardin, a new broad-spectrum antibiotic. I. Discovery and biological properties. In Antimicrob. Agents Chemother. 1962, pp. 583-591.
Oliver, T. J.; Sinclair, A. C.: Antibiotic M-319. Patent no. 3155582.3155582.
Paget, M. S.; Chamberlin, L.; Atrih, A.; Foster, S. J.; Buttner, M. J. (1999): Evidence that the extracytoplasmic function sigma factor sigmaE is required for normal cell wall structure in Streptomyces coelicolor A3(2). In J. Bacteriol. 181 (1), pp. 204-211.
Petkovic, H.; Raspor, P.; Lesnik, U.: Genes for biosynthesis of tetracycline compounds and uses thereof. EP2154249.
Pickens, L. B.; Tang, Y. (2010): Oxytetracycline biosynthesis. In J. Biol. Chem. 285 (36), pp. 27509-27515. DOI: 10.1074/jbc.R110.130419R110.130419.
Proctor, R.; Craig, W.; Kunin, C. (1978): Cetocycline, tetracycline analog. in vitro studies of antimicrobial activity, serum binding, lipid solubility, and uptake by bacteria. In Antimicrob. Agents Chemother. 13 (4), pp. 598-604.
Rasmussen, B.; Noller, H. F.; Daubresse, G.; Oliva, B.; Misulovin, Z.; Rothstein, D. M. et al. (1991): Molecular basis of tetracycline action. identification of analogs whose primary target is not the bacterial ribosome. In Antimicrob. Agents Chemother. 35 (11), pp. 2306-2311.
Rawlings, M.; Cronan, J. E., Jr. (1992): The gene encoding Escherichia coli acyl carrier protein lies within a cluster of fatty acid biosynthetic genes. In J. Biol. Chem. 267 (9), pp. 5751-5754.
Revill, W. P.; Bibb, M. J.; Hopwood, D. A. (1995): Purification of a malonyltransferase from Streptomyces coelicolor A3(2) and analysis of its genetic determinant. In J. Bacteriol. 177 (14), pp. 3946-3952.
Rhodes, P. M.; Hunter, I. S.; Friend, E. J.; Warren, M. (1984): Recombinant DNA methods for the oxytetracycline producer Streptomyces rimosus. In Biochem. Soc. Trans. 12 (4), pp. 586-587.
Sambrook, Joseph; Russell, David W. (2001): Molecular Cloning. a Laboratory Manual. 3rd. Cold Spring Harbor, N.Y.: Cold Spring Harbor Laboratory Press.
Stepanek, Jennifer J.; Lukežič, Tadeja; Teichert, Ines; Petković, Hrvoje; Bandow, Julia E. (2016): Dual mechanism of action of the atypical tetracycline chelocardin. In Biochim. Biophys. Acta 1864 (6), pp. 645-654. DOI: 10.1016/j.bbapap.2016.03.004.
Walsh, C. T.; Gehring, A. M.; Weinreb, P. H.; Quadri, L. E.; Flugel, R. S. (1997): Post-translational modification of polyketide and nonribosomal peptide synthases. In Curr Opin Chem Biol 1 (3), pp. 309-315.
Wang, P.; Zhang, W.; Zhan, J.; Tang, Y. (2009): Identification of OxyE as an ancillary oxygenase during tetracycline biosynthesis. In Chembiochem 10 (9), pp. 1544-1550. DOI: 10.1002/cbic.200900122.
Yin, Shouliang; Wang, Weishan; Wang, Xuefeng; Zhu, Yaxin; Jia, Xiaole; Li, Shanshan et al. (2015): Identification of a cluster-situated activator of oxytetracycline biosynthesis and
manipulation of its expression for improved oxytetracycline production in Streptomyces rimosus. In Microb Cell Fact 14, p. 46. DOI: 10.1186/s12934-015-0231-7.
Zhang, W.; Watanabe, K.; Wang, C. C.; Tang, Y. (2007): Investigation of early tailoring reactions in the oxytetracycline biosynthetic pathway. In J. Biol. Chem. 282 (35), pp. 25717-25725. DOI: 10.1074/jbc.M703437200.
Zhang, Wenjun; Ames, Brian D.; Tsai, Shiou-Chuan; Tang, Yi (2006): Engineered biosynthesis of a novel amidated polyketide, using the malonamyl-specific initiation module from the oxytetracycline polyketide synthase. In Applied and environmental microbiology 72 (4), pp. 2573-2580. DOI: 10.1128/AEM.72.4.2573-2580.2006.

1. A gene cluster encoding polypeptides involved in the biosynthesis of a tetracycline, wherein said gene cluster includes all of the nucleotide sequences (1) to (19):
(1) a nucleotide sequence encoding a polypeptide which comprises an amino acid sequence having at least 80%, such as at least 85%, sequence identity with the polypeptide of SEQ ID NO: 1 and which has the same functional property as the polypeptide of SEQ ID NO: 1 [ChdP];
(2) a nucleotide sequence encoding a polypeptide which comprises an amino acid sequence having at least 80%, such as at least 85%, sequence identity with the polypeptide of SEQ ID NO: 2 and which has the same functional property as the polypeptide of SEQ ID NO: 2 [ChdK];
(3) a nucleotide sequence encoding a polypeptide which comprises an amino acid sequence having at least 80%, such as at least 85%, sequence identity with the polypeptide of SEQ ID NO: 3 and which has the same functional property as the polypeptide of SEQ ID NO: 3 [ChdS];
(4) a nucleotide sequence encoding a polypeptide which comprises an amino acid sequence having at least 80%, such as at least 85%, sequence identity with the polypeptide of SEQ ID NO: 4 and which has the same functional property as the polypeptide of SEQ ID NO: 4 [ChdQI];
(5) a nucleotide sequence encoding a polypeptide which comprises an amino acid sequence having at least 80%, such as at least 85%, sequence identity with the polypeptide of SEQ ID NO: 5 and which has the same functional property as the polypeptide of SEQ ID NO: 5 [ChdQII];
(6) a nucleotide sequence encoding a polypeptide which comprises an amino acid sequence having at least 80%, such as at least 85%, sequence identity with the polypeptide of SEQ ID NO: 6 and which has the same functional property as the polypeptide of SEQ ID NO: 6 [ChdX];
(7) a nucleotide sequence encoding a polypeptide which comprises an amino acid sequence having at least 80%, such as at least 85%, sequence identity with the polypeptide of SEQ ID NO: 7 and which has the same functional property as the polypeptide of SEQ ID NO: 7 [ChdL];
(8) a nucleotide sequence encoding a polypeptide which comprises an amino acid sequence having at least 80%, such as at least 85%, sequence identity with the polypeptide of SEQ ID NO: 8 and which has the same functional property as the polypeptide of SEQ ID NO: 8 [ChdT];
(9) a nucleotide sequence encoding a polypeptide which comprises an amino acid sequence having at least 80%, such as at least 85%, sequence identity with the polypeptide of SEQ ID NO: 9 and which has the same functional property as the polypeptide of SEQ ID NO: 9 [ChdMI];
(10) a nucleotide sequence encoding a polypeptide which comprises an amino acid sequence having at least 80%, such as at least 85%, sequence identity with the polypeptide of SEQ ID NO: 10 and which has the same functional property as the polypeptide of SEQ ID NO: 10 [ChdMII];
(11) a nucleotide sequence encoding a polypeptide which comprises an amino acid sequence having at least 80%, such as at least 85%, sequence identity with the polypeptide of SEQ ID NO: 11 and which has the same functional property as the polypeptide of SEQ ID NO: 11 [ChdN];
(12) a nucleotide sequence encoding a polypeptide which comprises an amino acid sequence having at least 80%, such as at least 85%, sequence identity with the polypeptide of SEQ ID NO: 12 and which has the same functional property as the polypeptide of SEQ ID NO: 12 [ChdGIV];
(13) a nucleotide sequence encoding a polypeptide which comprises an amino acid sequence having at least 80%, such as at least 85%, sequence identity with the polypeptide of SEQ ID NO: 13 and which has the same functional property as the polypeptide of SEQ ID NO: 13 [ChdTn];
(14) a nucleotide sequence encoding a polypeptide which comprises an amino acid sequence having at least 80%, such as at least 85%, sequence identity with the polypeptide of SEQ ID NO: 14 and which has the same functional property as the polypeptide of SEQ ID NO: 14 [ChdR];
(15) a nucleotide sequence encoding a polypeptide which comprises an amino acid sequence having at least 80%, such as at least 85%, sequence identity with the polypeptide of SEQ ID NO: 15 and which has the same functional property as the polypeptide of SEQ ID NO: 15 [ChdA];
(16) a nucleotide sequence encoding a polypeptide which comprises an amino acid sequence having at least 80%, such as at least 85%, sequence identity with the polypeptide of SEQ ID NO: 16 and which has the same functional property as the polypeptide of SEQ ID NO: 16 [ChdOI];
(17) a nucleotide sequence encoding a polypeptide which comprises an amino acid sequence having at least 80%, such as at least 85%, sequence identity with the polypeptide of SEQ ID NO: 17 and which has the same functional property n as the polypeptide of SEQ ID NO: 17 [ChdOIII];
(18) a nucleotide sequence encoding a polypeptide which comprises an amino acid sequence having at least 80%, such as at least 85%, sequence identity with the polypeptide of SEQ ID NO: 18 and which has the same functional property as the polypeptide of SEQ ID NO: 18 [ChdOII]; and
(19) a nucleotide sequence encoding a polypeptide which comprises an amino acid sequence having at least 80%, such as at least 85%, sequence identity with the polypeptide of SEQ ID NO: 19 and which has the same functional property as the polypeptide of SEQ ID NO: 19 [ChdY].

2. The gene cluster according to claim 1, wherein said gene cluster further comprises at least one of the nucleotide sequences (20) and (21):
(20) a nucleotide sequence encoding a polypeptide which comprises an amino acid sequence having at least 80%, such as at least 85%, sequence identity with the polypeptide of SEQ ID NO: 20 and which has the same functional property as the polypeptide of SEQ ID NO: 20 [SARP/ChdB]; and
(21) a nucleotide sequence encoding a polypeptide which comprises an amino acid sequence having at least 80%, such as at least 85%, sequence identity with the polypeptide of SEQ ID NO: 21 and which has the same functional property as the polypeptide of SEQ ID NO: 21 [LuxR/ChdC].

3. The gene cluster according to claim 2, wherein said gene cluster comprises both nucleotide sequences (20) and (21).

4. The gene cluster according to any one of claims 1 to 3, wherein the nucleotide sequences (18) and (19) are linked to form a fusion protein of the respective polypeptides encoded by them.

5. The gene cluster according to claim 4, wherein the fusion protein comprises an amino acid sequence having at least 80%, such as at least 85%, sequence identity with the polypeptide of SEQ ID NO: 22 and has the same functional properties as the polypeptides of SEQ ID NO: 18 and SEQ ID NO: 19 [ChdOII+ChdY].

6. The gene cluster according to any one of claims 1 to 5, wherein the nucleotide sequences (7) and (17) are linked to form a fusion protein of the respective polypeptides encoded by them.

7. The gene cluster according to claim 6, wherein the fusion protein comprises an amino acid sequence having at least 80%, such as at least 85%, sequence identity with the polypeptide of SEQ ID NO: 23 and has the same functional properties as the polypeptides of SEQ ID NO: 7 and SEQ ID NO: 17 [ChdL+ChdOIII].

8. A DNA construct comprising the gene cluster according to any one of claims 1 to 7.

9. The DNA construct according to claim 8, wherein said DNA construct further comprises a nucleotide sequence encoding a polypeptide which comprises an amino acid sequence having at least 80%, such as at least 85%, sequence identity with the polypeptide of SEQ ID NO: 24 and which has the same functional property as the polypeptide of SEQ ID NO: 24 [OxyD].

10. A recombinant host cell comprising the gene cluster according to any one of claims 1 to 7 or the DNA construct according to claim 8 or 9, wherein the gene cluster or DNA construct is heterologous to said host cell.

11. The recombinant host cell according to claim 10, which heterologously expresses the polypeptides encoded by the gene cluster.

12. The recombinant host cell according to claim 10 or 11, which heterologously expresses a polypeptide which comprises an amino acid sequence having at least 80%, such as at least 85%, sequence identity with the polypeptide of SEQ ID NO: 24 and which has the same functional property as the polypeptide of SEQ ID NO: 24 [OxyD].

13. The recombinant host cell according to any one of claims 10 to 12, which is a bacterium.

14. A process for the biosynthetic production of a tetracycline, said process comprises the steps of a) cultivating a recombinant host cell according to any one of claims 10 to 13 in the presence of a suitable substrate under conditions conducive to the production of said tetracycline and, optionally, b) recovering the tetracycline from the medium employed in cultivation.

15. The process according to claim 14, wherein the tetracycline is chelocardin or an analogue thereof.


SEQUENCE LISTING (49 sequences)

SEQ ID NO: 1 - protein, 432 aa, Amycolatopsis sulphurea
MTGPSDAHRVVITGIGVVAPGDRGTKQFWELITAGRTATRPISLFDASSFRSRVAAECNFDPIAAGLSQRQIRKWDRTTQFCVVAAREAVADSGMLGEQDPLRTGVAIGTACGMTQSLDREYAVVSDEGSSWLVDPDYGVPQLYDYFLPSSMATEIAWLVEAEGPVGLVSTGCTSGVDVIGHAADLIRDGEADIMVAGASEAPISPITVACFDAIKATTARNHEPESASRPFDQTRSGFVLGEGAAVFVLEELRHAKRRGAHIYAEIVGYASRCNAYSMTGLRPDGREMADAIDGALNQARIDPSRIGYVNAHGSSTRQNDRHETAAIKTSLGAHAYQVPVSSIKSMVGHSLGAIGSLEVAACALTIEHSVIPPTANLHVPDPECDLDYVPLVAREQEVDVVLSVASGFGGFQSAILLTGPDGRTGKRVTQR

SEQ ID NO: 2 - protein, 418 aa, Amycolatopsis sulphurea
MSVETEPAPVPGRSTVRPVVTGLGVIAPNGMGTEAYWAATLRGDSGLRRITRFDPDGYPARIAGEVSFDPAGRLPDRLLPQTDHMTRLALIAAEEALADAGADPRNLPDYATGVMTAASGGGFEFGQRELQELWSKGGSYVSAYQSFAWFYPVNTGQISIRHGMRGSSGTLVSEQAGGLDAVAKARRHVRDGTPLMVTGGIDGSLCPWSWLCMLRSGRLSTASDPQRAYLPFDTEASGMVPGEGGALLVIEDPAAAQRRGVDRIYGQIAGYCATFDPGPGSRRPPGLRRAVEQALAEARLHPSEVDVVFADAAGLPDLDRAEIEVLVRIFGARAVPVTAPKTMTGRLLAGGSSLDLATALLSLRDKVIPPTVHIGKFGYRDEIDLVRDSPRQAPLSTALVLARGYGGFNSAMVLRGAT

SEQ ID NO: 3 - protein, 88 aa, Amycolatopsis sulphurea
MAEFTIAELVRLLRECAGEEEGVDLDGEVGDLPFDELGYDSLALFNTIGRIEREYTVDLPEDVVWQATTPGALVDLVNSSRTSPAAAD

SEQ ID NO: 4 - protein, 302 aa, Amycolatopsis sulphurea
MSHPEAEQTQASIVVDAPAEITYAMLVDVANWPLLYPWIAHTEFVERAPTEDLVQFWAVNPLGRIRIWTSRRYLDASALRMDIEQQGSVGPITGLTGSWTFKPLPGDRCLVESRHAFHAATPEDRAAGVTELNRHGKLQMETLKSRVENRTRLAELTWSFEDSLVIESELGQVYRALRDVGSWPAHLPCLTALEVTEDENDVQFYDVRTQDADEPSRFVRICLPDKGIAYKQLTVTAPVDLHLGRWTLTETPAGVAVTSAHTVLVNPSAAEQLPELRDRLHKTSSADSLAELQLVKRLAETR

SEQ ID NO: 5 - protein, 315 aa, Amycolatopsis sulphurea
MPAAAQQHTEHRIDIDAPAGLVYRIIADATEWPRHFTPTVHVDQSELDGHTERLHIWANANGQLKSWTSLRELDERAGRIRFRQEVSAPPVASMSGEWIVSERVAERTTLVLTHDFAAVDDDPAGVEWITKATNGNSDTELANIKALAERWERMDRLAFDFEDSVLVRAPKERAYHFLDRVDLWPDRLPHVARLELREDVPGVQHMSMDTKAKDGSTHTTVSVRVCFPEARIVYKQLVPPALLTTHTGVWTFEDTADGVLVTSAHTVVLNEANIGTVPGPAATVESTRDFVRNAISGNSQATLRHAKAFAEATDA

SEQ ID NO: 6 - protein, 150 aa, Amycolatopsis sulphurea
MSQAVQAVGSTEDIALYVEVQQFYGRQMRYLDEGRVQEWAKTFTEDGMFAANAHPEPARGRTAIEAGALEAATRLAEQGIQRRHWLGMVQVDPQPDGSIVAKSYAVIIGTPLGGKAAVDLSCDCVDVLVREGGALLVRERQVYRDDLPRN

SEQ ID NO: 7 - protein, 547 aa, Amycolatopsis sulphurea
MATPETTVAQDSTVSGDPLTGIAGFIGGPRVDDLLRSAAERAPEHVAIRAAACELSYAALDERAGRLARAVHAQLAEPGQVVALAGVLDPAFAVSFFGIARSGAVPALINPLLLADGLVHVLGTSGAVTAIVPPEVHRRLVPVLDRLPELRHLVLTHRDDQTEPSGPPTLDEVLASAPDGVLPATTCDENSVACLQFTSGTTGAAKAVRLSHRNITVNAAQSGHAHGITPSSVLFNYLPTFHLMHLTMAVTFAATLVLHVGDDVAQAVDAADNEKATHFYSLPMRLSRLAVHPRLSTLAADALQVILCGGSALPLPSTRALTGCFGVPVVQGYGLQETSPSTHFDSLSCPKTGSSGRPVAGTGCRIVDVDSRAVLPVGEKGEIQVRGPQLMLGYLGREPGQDVDPDGWFSTGDVGYVDAEGVLFVVDRIKDVFKCDNWLVSPTEIERVVLSHPEVADCVVLDYPDDFSGSVAYGLVVPKGAGLNPAQLAEFVAERLPYYAHLRHVELTDRIPRSPNGKLQRRALREQIHARNADGASEIARQNRSKT

SEQ ID NO: 8 - protein, 262 aa, Amycolatopsis sulphurea
MRSTEEPRVALVTGATSGIGLAVTKALAARGLRVFICARNRENVVSTVKELRAQGLDVDGQAGDVRSVAAVREVVESAVNRFGTISVLVNNAGRSGGGITAKITDELWQDVIDTNLNSVFTVTREVLTTGGLDGADGGRIINIASTGGKQGVPLGAPYSAAKSGVIGFTKALGKELAKTGVTVNAVCPGYVETPMAVRVRQAYASTWDTTDENVLARFNDKIPLGRYCTPEEVAGMVDYLVADTAASVTAQAINVCGGLGNY

SEQ ID NO: 9 - protein, 341 aa, Amycolatopsis sulphurea
MTGTVLPAAVMRVRELALSAACAASVRAAAKLGLADVLDDQPATVDELAKAVHADPGALRRLMRSLTCFEVFAEPEPDKFVHTDASRLLREDAPRSLKHILLWGTEPWTWELWPHLDQAVRTGKNVFDDLHGKDFFEYLHEQWPESAEVFDKAMTQSSKLSALAIADRLDLTGAERLADIAGGQGNVLATLLSRNEKLNGVLFDLPAVVAGADERLRVGGALADRAELVAGDCRREIPVQADVYLFKNILEWDDESTVLALRNAVAAGRPGARVVIIENLVDGTPEMKFATAMDLLLLLNVGGKKHTKDGLLGLIGQAGLQVDRVSAVNSYLHMVETTIPG

SEQ ID NO: 10 - protein, 343 aa, Amycolatopsis sulphurea
MTETEPTTVTAKKLHEIMRGYVKTALLRTAIELNIFDGIGDRTVDADGLARALGVDARGLRITLDSLAAIGLLRTVDGKYALPVDGDKFLLSSSPTFFGPSLKLGASDWEWDAQKRLTEAVRKGGAVMDSHALTPEFDYWEDFAENTTWFNNGAAELMAEQLLPWAKDRDSVDVLDVACSHGYYGVNLAKAEPKARVWGVDWPNVLPITAKNYERNGISDRFEGIPGDMFSVPLGGPYDVVMITNVLHHFSADTSTNLLRRLFDVLKPGGRIAVTGHTFVEGERPEDKPLPYLFSQIMLVMTDEGETHSTKTYERMFTDAGFVNPQIFTAEKAMHTVFTADKA

SEQ ID NO: 11 - protein, 448 aa, Amycolatopsis sulphurea
MTMTKAREDLTTGSAVTLELAGLHTGVSDPRMDSMRLLSETAFRYPQAVSFASGRPYEGFFDIAKLHHYLDRFVEHLRERGMPEERIKKALFQYGPINGLIRDMIARTLEVDEDIHVAPEAVMVTHGCQEAMMIALRGLFASPSDVLLTVSPCYVGIAGAAKMLDIPMAAVPEGAEGIDPEQVAAVAREVRASGLRPVACYVTPDFSNPSGHSLPIATRHRLLEVAAEQDLLLLEDNPYGLFGRDGAQVPTLKALDTQRRVIYLGSFAKTVFPGARVGYLVADQEVTGGPGAAKPLAEELGKVKSMFTVGTSGISQALVGGVLLDADFSLRTANRELADLYVRHLEVTLSSLAEHFPPERFAEHGVRWNVPGGGFFLSVEVPFVAGLEALDRSARDHGVGWAPMSMFYVGDGGEHIVRLGFSPLTAEEIREGVRRLAEFVKATPRTDR

SEQ ID NO: 12 - protein, 399 aa, Amycolatopsis sulphurea
MRLLLVTWNAPAHLFAMVPLGWAAQVAGHEVRVAAPPSCTEAIGRTGLTAVPVGTQRPAAPSGPPPGAPSGRWPVDWAVHPELLDDSRHELLRSLAARQFAAAEPMLDDLIEFARWWSPDVVVYDPTSLAGEVAATVLGVPAFACSWGRAAAVRIERGLGSEPLLGYARLFERFGCQAPQGPASWFDPFPAGLWLAEPDLPRQAMRFVPGTGGDAGSLPGWLREHSARPRICVTSAEPGGLLRPEAVRAFYRHALTVLSDVDAEVVVPAGPAARTLLAEIPHTARIVDPVAAHLLVPACRLTVHQGDGLSTLAGLNSGVPQYVLAPRPEQELVGHQLHRAGAGGYRSLSEPVDVPAERAVLDALLAPESGGAAARKLQEETLALPLPSAVLGRIESATR

SEQ ID NO: 13 - protein, 511 aa, Amycolatopsis sulphurea
MSVHGVMPSGWSSPVGRLLSQAGLGLLSWVVPPALVDEALAVAGRDERRFRALPSRLGVYFVLALCLLRTKSGNATIRAMFSQESLPRLSVLGWWPPASTALTKLRDRIGVVPFQLLFGALARAAPTRNRPWSHAFGLEVCAWDGTEVEPADTAANREHFPPHHRTGVARGPSKIRVLVLLSCGSRRLLGAVTGPLSQGEPTLAYQLLPRLHDRMLLLADRCFLGYPLWTAARERGAHLLWRAKQNTPKLPVQHALPDESWLSTLHAPADARRWARNVRRNKQRGHRPPTPRPINGIVVRVVEALITVTVDGVTRTEKYRLVTSLLDPAHAPAGQLVALYARRWTAETGIKEIKTTLLAKRPLRGHTPIRAQQELWATLIVYQAIRLLISHAALTQNLDPSRISFTSARDAAEHAITTTPADTSRHLQWVAQDLCRQLITVHTHHRVYPRALKRTTTRYPHRSKTPQPTSTKASYQVHILPTAETTPPTTTKPTPHQPRTDLSSWHWTQSP

SEQ ID NO: 14 - protein, 481 aa, Amycolatopsis sulphurea
MKDNLARPDTVGADENRISPALWGLASILILGGFTSMFTSTIVNVALDTLSQKLSAPLGTVQWTATGYLMALATAVPVSGWASKRYGATRLWLGSVALFTLFSALCALSTSVEMLITFRVLQGIAGGLLVPAGQILLVTAAGPKRIGRMLTAVSVPIYLAPAVGTTLGSVLTQGLGWPWLFWITVPLGALGFFAGLRWLPKAPPKGAPALDVRGLIILVAGLPLLTYGVAGIGENGGRTETIAVIAAVAGALLLALFTLHAVRSRNPLLNLRLFKDRAFSSAAVVIFCMGIALFGAMIVLPIYFLQVRHEDLVTAGLLTAPSAIGTVLALPLAGKMTDKIGGARVIFAGLVVTIIGTIPLALVTPHDSYVWLSLVQIVRGIGIGMTTTPAMAAGLAMIGKEDVPHATPIFNVLQRVGGSFGTALTTVLVAFQLASGPQTDEGAADAIGYTHWWIVACTAIVLIPSMLLVQVESRRRQAAAA

SEQ ID NO: 15 - protein, 190 aa, Amycolatopsis sulphurea
MRLSPETFARAALKLLNKSGLEGVSLRKLGDELGVQGPALYAHFKNKQELLDLMAEIMLDEALAPLDAMTEVADWHWWLAERARTIRRTLLSYRDGALLHAGSRPTADGAEAIPALLRPLREAGFSDKEALTVIITIGRYTLGCVIDEQRPGEPAPQPGPGADDTFEFGLQALLAGLRARLPERVPDSAG

SEQ ID NO: 16 - protein, 404 aa, Amycolatopsis sulphurea
MTDIRTDFCVVGGGPAGLTLALLLARSGVRVVVVERSRSFDREYRGEILQPGGQALLAELGVLTPAREHGAHEHHRFLLEEHGKVLINGDYRRLPGPFNCLLSIPQRHLLRELLAQCHEHAGFQYLSGTKVTGLVEDGGRVRGVVCGDDQVVLAHCVIGADGRYSKVRQLAGIPADRVEGFRQDVLWFKLSADGELPSEVRVFRAGGNPVLAYTSVRDRVQFGWTLPHKGYQLLAQQGLAHIKEQLRAAVPGYADRIDEEITSFRDLSLLDVFSGGARQWVRDGLLLIGDSAHTHGPIGAQGINLAIQDAVAAHPLLLESLRANDSSGAMLGRFVTGRKRDIDRMNRIQAVQGKAMLSAGRVSSVVRPRLAMVVARTPIYRAMLRQIAFGNTGIRIRAELFARR

SEQ ID NO: 17 - protein, 96 aa, Amycolatopsis sulphurea
MFTFINRFTVTGDATEFRRLLGQITAHMTAQPGFRSHRLYQSARDEAVFTEIAEWDSAEDHQRATAGKGFREPVGEAMKHATAEPAPFVLRAEHGA

SEQ ID NO: 18 - protein, 550 aa, Amycolatopsis sulphurea
MPEDSGEEPEVLVAGAGPVGLTAAHELARRGVRVRLVDRSAGPATTSRALATHARTLEIWHQMGLLGELLPRGRRVEHFTLHLKGKTLMCFDTNYDTMPTRFPFSLMVDQVVTEEVLRRQVRALGVTVEWGVELTWFDQEPDGVLAELRHADGTVEQVTAAWLVGADGARSTVRKRLDLRLQGDSTQTWLNADVVLDTDLAGDSNHLLHTGRGTLLLVPFPEPGKWRVVDTEDTDHADDARIVRARLADKLTRALGRPIEVPEPSWISVFTVQQRMIDRMRAGRCFVAGDAAHVHSPASGQGMNTGIQDAYNLAWKLADVVRGHAKESLLDSYGAERVPIGETLLRTTRTATALVSLRNTVAPLVMPAGTRLLGALKPLKRRIERTMIRGFCGLTLNYTHSPLSLACATPDGIQPGHRVGCSVDRARTSPGWQGLVTELTDPRWTLLAFADSQEQRQIAAQVERRYGKAVSVRVVAEAATSERVLADPGDDLARDFAMRAGYFVLIRPDGHLAAKGRLSDDLDGAFGALGLVPADAGGDPAHHLDSEGSR

SEQ ID NO: 19 - protein, 256 aa, Amycolatopsis sulphurea
MRIIDLSATMDAADRWEANPVTHEVLTAAEGAQHMAAEMKEHFGIDFDPSVLPGGELLTLDTLTLTTHTGTHVDAPSHYGTPRDGVARHIDQMPLEWFLRPGVVLDLTGEPVGAAGADRLREEFERIGYTPKPLDIVLLNTGADALAGSPKYFTDFTGLDGKATELLLDLGVRVIGTDAFSLDAPFGHMIAEYRRTGDRSVLWPAHFAGRDREYCQIEGLTNLAALPSPTGFSVSCLPVKIAGAGAGWTRAVALLD

SEQ ID NO: 20 - protein, 258 aa, Amycolatopsis sulphurea
MKFNLLGPMEVLCADGTVTPSAAKMRWILALLLLHGNRVVDQASMIDELWGDHPPRSAVTTTQTYVYQLRKKYDYYAQREGRKSFIVTRAPGYLLQLDDDQLDVRRFQRLSAEGSALFSAGHAERADEVLRQALRLWRGPALAGIAPGRMLQAHVAYLEEARLRTVQVRILADAALGRHRDLIPELRSLVIEHPLDEWFHQQLITALAEAGRRGDALHACRVLHRTLADELGVAPSEPLRKLQQDLLTGHVRRAPAHV

SEQ ID NO: 21 - protein, 212 aa, Amycolatopsis sulphurea
MITDRVRILIVEESSVFRVGLLSLIQGADDLAAIDAVATIGEALDSVSRGIVDVILYGVDGWGPEVESGLLELSAAAPGKPVVILSQENRFGFIQEFLGIGVRGYLPKNVSDMYLLSVVREVSRDERCVFLSVPSVDVRSLSSNLRTPLSRREHEIMSLVARGMTNAQIGNCLAITQGTVKRHLRNIFVKLNAVSRLDAVNKAQTAAPLVPA

SEQ ID NO: 22 - protein, 806 aa, Amycolatopsis sulphurea
MPEDSGEEPEVLVAGAGPVGLTAAHELARRGVRVRLVDRSAGPATTSRALATHARTLEIWHQMGLLGELLPRGRRVEHFTLHLKGKTLMCFDTNYDTMPTRFPFSLMVDQVVTEEVLRRQVRALGVTVEWGVELTWFDQEPDGVLAELRHADGTVEQVTAAWLVGADGARSTVRKRLDLRLQGDSTQTWLNADVVLDTDLAGDSNHLLHTGRGTLLLVPFPEPGKWRVVDTEDTDHADDARIVRARLADKLTRALGRPIEVPEPSWISVFTVQQRMIDRMRAGRCFVAGDAAHVHSPASGQGMNTGIQDAYNLAWKLADVVRGHAKESLLDSYGAERVPIGETLLRTTRTATALVSLRNTVAPLVMPAGTRLLGALKPLKRRIERTMIRGFCGLTLNYTHSPLSLACATPDGIQPGHRVGCSVDRARTSPGWQGLVTELTDPRWTLLAFADSQEQRQIAAQVERRYGKAVSVRVVAEAATSERVLADPGDDLARDFAMRAGYFVLIRPDGHLAAKGRLSDDLDGAFGALGLVPADAGGDPAHHLDSEGSRMRIIDLSATMDAADRWEANPVTHEVLTAAEGAQHMAAEMKEHFGIDFDPSVLPGGELLTLDTLTLTTHTGTHVDAPSHYGTPRDGVARHIDQMPLEWFLRPGVVLDLTGEPVGAAGADRLREEFERIGYTPKPLDIVLLNTGADALAGSPKYFTDFTGLDGKATELLLDLGVRVIGTDAFSLDAPFGHMIAEYRRTGDRSVLWPAHFAGRDREYCQIEGLTNLAALPSPTGFSVSCLPVKIAGAGAGWTRAVALLD

SEQ ID NO: 23 - protein, 643 aa, Amycolatopsis sulphurea
MATPETTVAQDSTVSGDPLTGIAGFIGGPRVDDLLRSAAERAPEHVAIRAAACELSYAALDERAGRLARAVHAQLAEPGQVVALAGVLDPAFAVSFFGIARSGAVPALINPLLLADGLVHVLGTSGAVTAIVPPEVHRRLVPVLDRLPELRHLVLTHRDDQTEPSGPPTLDEVLASAPDGVLPATTCDENSVACLQFTSGTTGAAKAVRLSHRNITVNAAQSGHAHGITPSSVLFNYLPTFHLMHLTMAVTFAATLVLHVGDDVAQAVDAADNEKATHFYSLPMRLSRLAVHPRLSTLAADALQVILCGGSALPLPSTRALTGCFGVPVVQGYGLQETSPSTHFDSLSCPKTGSSGRPVAGTGCRIVDVDSRAVLPVGEKGEIQVRGPQLMLGYLGREPGQDVDPDGWFSTGDVGYVDAEGVLFVVDRIKDVFKCDNWLVSPTEIERVVLSHPEVADCVVLDYPDDFSGSVAYGLVVPKGAGLNPAQLAEFVAERLPYYAHLRHVELTDRIPRSPNGKLQRRALREQIHARNADGASEIARQNRSKTVFTFINRFTVTGDATEFRRLLGQITAHMTAQPGFRSHRLYQSARDEAVFTEIAEWDSAEDHQRATAGKGFREPVGEAMKHATAEPAPFVLRAEHGA

SEQ ID NO: 24 - protein, 612 aa, Streptomyces rimosus
MCGIAGWIDFERNLAQERATAWAMTDTMACRGPDDAGLWTGGHAALGHRRLAVIDPAHGRQPMHSTLPDGTSHVITFSGEIYNFRELRVELESQGHRFRTHCDTEVVLHGYTRWGRELVDRLNGMYAFAVWDEARQELLLVRDRMGVKPLYYHPTATGVLFGSEPKAVLAHPSLRRRVTAEGLCEVLDMVKTPGRTVFSGMREVLPGEMVTVGRSGVARRRYWTLQAREHTDDLETTIATVRGLLTDRVRRQLVSDVPLCTLLSGGLDSSAVTALAARAGDGPVRTFSVDFSGAGTRFQPDAVRGNTDAPYVQEMVRHVAADHTEVVLDSADLAAPEVRAAVLGATDLPPAFWGDMWPSLYLFFRQVRQHCTVALSGEAADELFGGYRWFHRTAAIDAGTFPWLTAGSARYFGGRGLFDRKLLDKLDLPGYQRDRYAEARKEVPVLPGEDAREAELRRVTYLNLTRFVQTLLDRKDRMSMATGLEVRVPFCDHRLVDYVFNVPWAMKSFDGREKSLLRAAVRDLLPESVVTRVKTPYPATQDPVYERLLRDELAALLADSQAPVRELLDLGRARDLLRRPVGAVSQPYDRGSLELVLWMNTWLAEYGVSLEL

SEQ ID NO: 25 - protein, 340 aa, Streptomyces rimosus
MTADTKATGAPAARAPRPVALLLPGQGSQYRRMAAGLYAAEPVFAEAVDEVLGAMGAEGARMHADWLAERPELPVDHVLRAQPLLFAVDYALGRLVTSWGIRPVALLGHSIGEMAAATLAGVFTVRDAARVVLDRVTRLTAAPPGGMLAVAESAASLEPFLGDGVVVGAVNAPRQTVLGGPEDALRAVGETLRARGITAQRVPALSPFHSPAIAPHARGAEAVLATVERRPPRTVVHSCYTAAPLTAQQVADPAYWAAHPVDQVRFWPALDGLLAPGGLVVVEAGPGRTLSSLALRHPSVRRGDCMVVPLSPKRADGPEDDRVALGEAVDALRGEGYRIP

SEQ ID NO: 26 - DNA, 22747 bp, Amycolatopsis sulphurea
ccgatgcgcggtcgccccacccggcttccagggcggcttgaggatcgccgggcaggctctctgctggtgactgggctgaccggcgacagagggagacgcggtgacaggaccaagcgatgcccatcgtgtcgtgattaccgggattggggtggtcgccccgggcgaccgtggtaccaagcagttctgggagctgatcacggcgggccgtacggccacccggccgatctcgttgttcgacgcgagttccttccggtcgagggtggccgccgagtgcaacttcgatccgatcgccgccgggctgagccagcggcagatccggaaatgggaccgcaccacgcagttctgcgtggtggcggcaagggaggcggtggccgacagcgggatgctcggggagcaggatcccctgcgtacgggggtggcgatcggcaccgcctgcggcatgacccagagcctggaccgcgagtacgcggtggtcagcgacgagggcagcagctggctggtcgacccggattacggggtgccccagctctacgactacttcctgccgtcctcgatggccaccgagatcgcgtggctggtggaggcggaaggaccggtggggctggtttccaccggatgcacgtcgggggtcgacgtgatcgggcacgccgccgacctgatccgcgacggcgaggccgacatcatggtcgccggtgcctccgaggcgccgatctcgccgatcacggtggcctgcttcgacgcgatcaaggccaccacggcacgcaaccacgaacccgagtcggcttccaggccgttcgaccagacccgcagcgggttcgtgctcggcgagggcgcggccgtcttcgtgctggaggagctgaggcacgccaagcggcggggcgcgcacatctacgccgaaatcgtcgggtacgcgtcccggtgcaacgcctacagcatgaccgggttgcgtccggacggccgcgagatggccgacgcgatcgacggggcgttgaaccaggccaggatcgacccgtcccggatcggctacgtcaacgcccacgggtcgtcgaccaggcagaacgatcggcacgagaccgcggccatcaagaccagcctcggagcgcacgcctatcaggtgccggtcagctcgatcaagtcgatggtcgggcattcgctcggcgcgatcggctcgctggaggtcgcggcctgcgcgctgaccatcgagcattcggtgatcccgccgacggcgaatctgcacgtgcccgacccggagtgcgacctggactacgtgccactggtggcgcgggaacaggaggtcgacgtggtgctcagcgtcgcgagcgggttcggcggtttccagagtgccatcctgctgaccggcccggacggcagaaccgggaagcgggtaacgcagcgatgagcgtggagacagaaccggcgccggtgcccggccgcagcacggtacggccggtggtgaccgggttgggcgtgatcgcgcccaacggtatgggcaccgaggcctactgggcggcgacccttcgtggcgacagcggactgcggcggatcacccggttcgacccggacggctacccggcccggatcgcgggcgaggtcagcttcgacccggccgggcggctgcccgaccggctgttgccgcagaccgaccacatgacccggctcgcgctgatcgccgcggaggaggcactcgccgacgcgggtgccgatccgcggaacctgcccgactacgcgaccggggtcatgaccgcggcctcgggcggcggattcgagttcgggcaacgggaactgcaggagctgtggagcaaaggcggctcctacgtcagcgcgtaccagtccttcgcctggttctacccggtgaacaccgggcagatctcgatccggcacgggatgcgcggctccagcggcacgctcgtgtccgaacaggccggtgggctggacgcggtcgccaaggcccgcaggcacgtgcgcgacggaacaccgctgatggtgaccggcggcatcgacggatcgctgtgcccgtggtcctggctgtgcatgctgcgctccggccggttgagcacggcgagtgatcctcagcgcgcctatctgcccttcgataccgaggcttccgggatggtgccgggtgagggtggcgcgttgctggtgatcgaggatcccgccgcggcacaacggcgtggcgtggaccggatctatgggcagatcgccggctattgcgcgacattcgaccccggcccgggttcacgtcgtccgcccggcctgcgccgggcggtcgagcaggcgctggccgaagcacggctgcatccgtccgaagtggacgtggtgttcgccgacgccgccggtctgcccgacctcgaccgggccgagatcgaagtgctggtccggatcttcggcgcgcgggccgtcccggtcactgcacccaagacgatgaccggccggctgctggccggtggaagctcgctggacctggccacggcgttgctctccctgcgggacaaggtcattccgcccaccgtccacataggaaagttcggttaccgggacgagatcgacctggttcgcgacagcccccggcaggcgccgctgtcgactgcgctggtgctggcccgagggtatggcgggttcaactccgcgatggtgcttcgcggtgccacctgatccccacacgaaaggaaggcaaccatggcagagttcacgatcgccgagctggtgcggctgctgcgcgaatgcgccggagaggaggaaggcgtggacctcgacggcgaggtcggggacctgccgttcgacgaactcggctacgactccctcgcgctgttcaacaccatcgggcggatcgagcgtgagtacactgtggacctccccgaggacgtagtgtggcaggcgaccacgccgggagcgctggtcgatctggtcaacagcagtcgcacgtcgcccgctgccgccgactgaaaaggctgtgaagggccccttcacggactcagagtccgtgaaggggcccttcacggactcgtctcgcccgttcagcgggtttctgccagccgcttgaccagctgcagctcggccaggctgtccgcactcgacgtcttgtgcagccggtcacgcaattcagggagctgttcggctgccgacgggttcaccagcaccgtatgcgcggacgtgacggcgactcccgcaggcgtctcggtcagggtccaccggccgaggtgcaggtccaccggtgcggtcacggtcagctgcttgtacgcgattcccttgtccggcaagcagatgcggacgaaccggctcggctcgtccgcatcttgcgtgcgcacgtcgtagaactgcacgtcgttctcgtcctcggtgacctcaagcgcggtgaggcagggcagatgtgcaggccaggacccgacgtctcgcagcgcgcggtagacctgcccgagttcgctctcgatcaccagcgaatcctcgaacgaccaggtcagctccgcgagccgggtacggttctcgacccggctcttgagggtctccatctgcagcttgccgtgccggttcagttcggtgaccccggccgcgcgatcctccggcgtggcggcgtggaaggcgtgccgggactccaccaggcaccggtcacccggcagcggtttgaaggtccacgacccggtcagcccggtgatcgggccgaccgagccctgctgctcgatgtccatccgcagcgcgctcgcgtccaggtaccggcgggaggtccagatccggatcctgcccagcgggttgaccgcccagaactgcaccagatcctcggtcggcgctcgctccacgaactcggtgtgcgcgatccacgggtagagcagcggccagttcgcgacgtcgaccagcatggcgtaggtgatctcggcgggcgcgtccacgacgatactggcctgcgtctgctcggcctcggggtgtgacatgggttctcctcgcgaatggggtgtgggaaagagggcgttcaccgggcggacccggcgaacgccctcgtaggaaacgaaaacggcgggttcaggccttgtctgcggtgaataccgtgtgcatggccttctccgcggtgaaaatctgcgggttgacgaaaccggcgtcggtgaacatgcgctcgtaggtcttggtcgagtgtgtctcgccttcgtcggtcatcaccagcatgatctgcgagaacaggtacggcagcggcttgtcctccgggcgctcgccctcgacgaaggtgtgcccggtgaccgcgatccgcccacccggcttgagcacgtcgaacagcctgcgcaacaggttcgtcgacgtgtccgccgagaaatggtgcagcacattggtgatcatcacgacgtcgtagggaccgccgagcgggacgctgaacatgtcgcccgggattccctcgaaccggtcgctgatcccgttgcgctcgtagttcttcgcggtgatcggcagtacgttcggccagtccacgccccagacccgggccttcggctcggccttggccaggttgacgccgtagtagccgtggctgcaggccacgtcgagcacgtcgacgctgtcgcggtccttggcccagggcagcaactgctccgccatcagttcggccgcaccgttgttgaaccaggtggtgttctccgcgaagtcctcccagtagtcgaattccggggtcagggcgtggctgtccatcacggcgccgcccttgcgcaccgcctcggtgagccgcttctgcgcgtcccattcccagtcgctggcgcccagcttcaacgacggcccgaagaaggtcgggctggagctgagcaggaacttgtcgccgtccaccggcaacgcgtacttcccgtcgaccgtgcgcagcaacccgatggctgccaacgagtccagcgtgatgcgcagtcctctggcgtccaccccgagcgcacgcgccaggccgtcggcgtcgacggtacggtcgccgatgccgtcgaagatgttcagctcgatcgccgtgcgcagcagtgccgttttcacatagccgcgcatgatttcgtgcagcttcttcgcggtcactgtggtcggttccgtctcggtcatgaccgtcctctcgtcgatccccttgatcccgcaagccatagtgctcagggcggctagtgcagcaatagagcggacggccacggatggacggctccacgagtccgggtcgagcctgcctcggcaggctggcgaaaccaccgacccgggacaagaaggagcggacatgcggctgttgctggtcacttggaatgcgccggctcacctgttcgcgatggtgcccctcggctgggccgcgcaagtagccgggcacgaggttcgcgtcgcggctccgccctcctgtaccgaggcgatcggccgtaccggcctgaccgccgtacccgtcggcacgcagcgtccggccgcgccgtccgggcctccgccgggagcgcccagcggtcgctggccggtcgactgggcggtgcatcccgaactgctcgacgacagccggcacgagcttctgcgttcgctggccgcgcgccagttcgccgccgccgagccgatgctggacgacttgatcgagttcgcacgctggtggtcgccggacgtcgtggtgtacgacccgaccagcctggccggcgaagtggcggccaccgtgctcggcgtgcccgccttcgcctgttcctggggccgcgcggcggccgtccggatcgagcggggtctgggctccgagccgctgcttggctacgcgcggctgtttgaacggttcgggtgccaggccccgcagggacccgcctcctggttcgacccgttccccgccggtctgtggctggctgaaccggatttgccgcggcaggcgatgcgcttcgtgcctggaaccggcggcgacgccggttcgctacccggctggctccgggaacactcggctcggccgaggatctgcgtcaccagcgcggaacccggtggtctgttgcggccggaagccgtacgggcattctaccggcacgcgctcaccgtactgtccgatgtggacgctgaggtcgtggtgcccgcgggtccggcggcccgcacgctgctggccgagattccgcacaccgcgcggatcgtcgacccggtggccgcgcatctgctcgtcccggcctgccggctcaccgtgcaccagggtgacgggctgtccactttggccggtctgaattcgggggttccccagtacgtcctggcgccacgtccggagcaggagctggtggggcaccagctgcaccgagccggtgcgggcggctaccggagcctgtccgaacccgtggacgttccggccgaacgtgcggtgctggatgccttgctggcaccggaaagcgggggagcggcggcccgcaagctgcaggaggaaacgctggcactcccgctcccgtccgccgtgctcggccggatcgagtccgccacccgctgagcctgcgctcggcctgcgcgccgagcccccgaccaaggctgtgaagggccccttcacggactcagagtccaatgccaggaacttaaggtctggtgggtggtgttgatcacggtttgggtgtgatcggcgtggtggtgtggataggcaacgaggctccggctagagctggtgattgtcgagatcatcagcaagccaggagcctcgttgtccgttcatggtgtcatgccctcgggctggtcctcgccggtgggtcggttgttgtcacaggccgggctggggttgctgtcgtgggtcgtcccgcccgcgctggttgatgaggcgctggccgtcgccggccgggacgaacggcggttccgggcactgccgtcgcggttgggcgtgtacttcgtgctcgcgttgtgcctgctgcgcaccaagtccggcaacgcgacgatcagggcgatgttctcgcaggagagtctgccccggttgtccgtgctgggctggtggccgccggccagcacggcgctgaccaagctgcgggaccggatcggcgtggtgcccttccagttgttgttcggcgcgctggcgcgggcggcgcctacccggaacaggccgtggtcgcacgcgttcgggctggaggtgtgtgcctgggacggcaccgaggtcgagccggccgacaccgcggccaaccgtgaacacttcccgccccatcaccgcacaggcgtggcccgcgggccgtccaagatccgggtgctggtgctgctgtcgtgtggcagccgccgtctgctcggcgcggtcaccggcccgctgagccagggcgagcccaccctggcctatcagctgctgccccggctgcacgaccggatgctgctgctggccgaccgctgcttcctcggctatccactgtggaccgcggcgcgggaacggggcgcgcatctgctgtggcgggccaagcagaacaccccgaagctgcccgtgcagcacgcgttgccggacgagtcctggctgtccaccctccacgccccggccgacgcccgccgctgggcgcgcaacgtgcgccgcaacaagcaacgcgggcaccgcccgcccacgccccgcccgatcaacggcatcgtcgtccgggtggtcgaagcactgatcaccgtcaccgtcgacggcgtcacccgcaccgagaaataccggctggtcaccagcctgctcgaccccgcacacgcacccgccggccagctcgtcgccctctacgcccgccgctggaccgccgaaaccggcatcaaagagatcaaaaccacgctgctggccaagcggcccctgcgcggccacaccccgatccgcgcccagcaggaactctgggccaccctgatcgtctaccaggccatccggctgctgatcagccacgcagcgctcacccagaacctcgacccgtcccggatctccttcacctccgcccgcgacgccgccgaacacgcgatcactaccacacccgccgacacatcccgacacctccaatgggtggcccaggacttatgccgacagctgatcaccgtccacacccaccaccgcgtctacccccgagcactcaaacgcaccaccacgcgctacccccaccgcagcaaaaccccgcaaccgaccagcaccaaagccagctaccaggtccacatcctgcccacagcagaaacaacaccacccaccacaaccaaaccaacaccacaccaacccagaaccgacctaagttcctggcattggactcagagtccgtgaaggggcccttcacagcccttcacggttggtcgaggcgcgccacggcctgcccggaggccacgaacaggccgccgccaggagtgggggttcccctggcggcggcccggacgagatcccccatgccgggggacctccgcatcaggcggccgcggcctgcctgcgacggctttccacctgtacgaggagcatcgacgggatgagcacgatcgcggtgcacgcgacgatccaccagtgcgtgtagccgatcgcgtcggccgcgccctcgtccgtctgcggacctgaggccagctggaaggccaccagcaccgtcgtcagcgcggtcccgaacgagccgccgacccgttgcagcacattgaagatcggggtggcgtgcggcacgtcctccttgccgatcatggccagcccggcggccatcgccggcgtggtggtcatcccgatcccgatgccgcgcacgatctggaccagcgacagccacacgtaactgtcgtgcggggtgaccagcgcgagcgggatggtgccgatgatggtgacgaccaggccggcgaagatgacccgggcgccgccgatcttgtcggtcatcttgcccgccagcggcagcgccagcacggtgccgatcgccgacggcgcggtcagcagaccggcggtgaccagatcctcgtgccgcacctggaggaagtagatcggcagcacgatcatcgcgccgaacagcgcgatccccatgcagaagatcacgaccgccgccgaggagaaggcccggtccttgaacagccgcaggttcagcagcggattcctcgagcgcaccgcgtgcagggtgaacagcgccagcagcagcgccccggccacggcggcgatcaccgcgatggtctcggtgcggccgccgttctccccgatcccggccacgccgtaggtcagcagcggcaggccggccaccaggatgatcaggccgcgcacatccagcgcgggcgcacccttcggcggcgccttcggcagccagcgcaggccggcgaagaagccgagcgcgccgagcggcacggtgatccagaacagccacggccagccgaggccctgggtcagcacgctgcccagcgtggtgcccaccgcgggcgccaggtagatcggcacgctgaccgcggtcagcatccggccgatccgcttcggcccggccgcggtgaccagcaggatctgcccggccggcaccagcaggccgcccgcgatcccctgcaggacccggaaggtgatcagcatttccaccgaggtcgacagcgcgcacagcgccgagaacagggtgaacagtgccacgctgcccagccacagccgggtcgccccgtagcgcttggacgcccacccgctgaccgggaccgcggtggccagcgccatcaggtacccggtcgccgtccactgcaccgtgcccagcggggcgctcagcttctggctgagggtgtccagggcgacgttgacgatggtcgaggtgaacatggacgtgaacccgccgaggatcaggatcgacgccagtccccacagcgccgggctgatccggttctcgtcggcgccgaccgtgtccggtctcgcgagattgtccttcatcgagcttccttgcgtgagtcgtcgtggtggtgcgatccgtgtccgcccggtgaccgagcgtcgacttagcaccgctaagtcttagcggtgctaatatggcccccgagacaccgatacgtcaagtcggtgcacgcaacccgaggggatgccgtgcggctgagtcccgaaaccttcgcgcgcgccgcgctgaagctgctcaacaaatccggcctggagggggtgagcctgcgcaagctcggcgacgagctgggcgtgcaggggcccgcgctgtacgcgcacttcaagaacaagcaggagctgctcgacctgatggccgagatcatgctcgacgaggcgctcgccccgctggacgcgatgaccgaggtggccgactggcactggtggctggccgaacgggccaggaccatccggcgcaccctgctgtcctatcgggacggtgcgctgctgcacgcgggatcccggccgaccgccgacggcgccgaagcgatcccggccctgctgcggccgttgcgagaggcgggcttcagcgacaaggaagcactcaccgtgatcatcaccatcggccgctacacgctggggtgtgtgatcgacgagcagcggccgggcgagcccgccccgcagcccggcccgggtgccgacgacaccttcgagttcgggctccaggcgttgctggccggactgcgggcccggctgccggaacgggtacccgacagcgccggataactgccgggagggttcagcggtcggtgcgcggggtcgccttgacgaactcggccagcctgcgcaccccctcgcggatctcctcggcggtcagcgggctgaagccgagccggacgatgtgctcgccgccgtcaccgacgtagaacatgctcatcggcgcccagcccaccccgtggtcgcgggccgagcggtccagtgcttcgaggccggcgacgaagggcacctcgacggagaggaagaacccgccgcccggcacgttccagcgcacgccgtgctcggcgaaccgctccggcgggaagtgttcggcgaggctgctcagcgtcacctccaggtgccgcacgtacaggtcggccagctcccggttcgcggtgcgcaggctgaaatccgcgtccaggagcaccccgccgaccagggcttgcgagatgcccgaggtgccgacggtgaacatgctcttgaccttgccgagttcctcggcgagcggcttggccgcgccggggccgccggtcacttcctggtctgcgaccaggtagccgacgcgtgcgccggggaagacggtcttggcgaaggagccgaggtagatcaccctgcgctgggtgtcgagtgccttgagcgtcggcacctgcgcaccgtcccggccgaacagaccgtacggattgtcctccagcagcagcaaatcctgctcggcggcgacttcgagcagccggtgccgggtggcgatcggcagcgagtgtcccgaggggttggagaagtccggggtcacgtagcaggccaccgggcgcagaccggacgcgcgtacctcgcgggccaccgccgcgacctgctcgggatcgataccctcggcgccctccggaaccgcggccatcgggatgtcgagcatcttcgcggctcccgcgatgccgacgtagcaaggcgaaacggtcagcaggacgtcgctcggcgaggcgaacagcccgcgcagcgcgatcatcatcgcttcctggcagccgtgggtcaccatgaccgcctccggcgcgacgtggatgtcctcgtccacctccagcgtgcgcgcgatcatgtcccggatcagcccgttgatcggcccgtactggaacagcgccttcttgatccgttcctcgggcatgccccgctcacgcagatgctcgacgaagcggtccaggtagtggtgcagcttggcgatgtcgaagaaaccttcatacggccggccggaggcgaacgagaccgcctgcggataccggaaagccgtctcgctgagcaaccgcatcgagtccatccgcggatcggacacccccgtgtgcaggccggccagttcgagcgtcacggccgagccggtggtcaggtcttcccgagccttcgtcatggtcatccagccttctgatgggggagcggcgacggtggccactgtggactgctcgcctcaagaggtggtctcgcccggttggaacggcgagtgccgggatccgaccgacaatcgagccgtgccggttacggtcccggcatgacggatatccggactgacttctgcgtcgtcggtggcggaccggccgggctgaccctggcgctgctgctggcccgatccggcgtacgggtcgtcgtggtcgaacggtcccgttccttcgatcgggaatatcgcggcgagatcctgcaaccgggtggccaggccttgctcgccgagctgggcgtgctgactccggcccgggagcacggagcgcacgagcatcaccgatttctcttggaggagcacggaaaagtcctgatcaacggcgactaccggcggctgcccggcccgttcaactgcttgctcagcatcccgcaacggcacctgctgagggaactgctggcgcagtgtcacgagcacgcgggcttccagtacttgtccggcacgaaggtcaccggtctggtcgaggacggtggccgggttcggggcgtggtctgtggcgacgaccaggtggtgctcgcgcactgcgtgatcggggccgacggacggtattcgaaggtgcggcagctcgccgggattcccgccgaccgggtcgagggcttccgtcaggacgtgttgtggttcaagctgtccgccgacggcgagctgccgagtgaggtgcgggtgttccgggcgggcggcaacccggtgctggcctacacgtcggtacgtgaccgcgtgcagttcggctggacgttgccgcacaagggttatcagctgctggcgcagcagggcctcgcgcacatcaaggagcagctgcgggccgccgtgcccggctacgcggaccggatcgacgaggagatcaccagcttccgggacctttccctgctggacgtcttctccgggggcgcccggcagtgggtgcgcgacggtctgctgctgatcggcgacagtgcgcacacccacggcccgatcggcgcgcaggggatcaacctggccatccaggatgctgtggccgcacatcccctgctgctggagtcgttgcgtgccaacgactccagcggggcgatgctcggccggttcgtgaccgggcgcaagcgggacatcgaccggatgaaccggatccaggccgtccagggcaaggcgatgctgtccgcgggccgggtctcgtcggtggtccggccgaggctggcgatggtggtcgcccgcaccccgatctaccgcgcgatgttgcggcagatcgcgttcggcaacaccgggatccggatccgcgccgagttgttcgcccgccgctgaccggtgatcagcccgggatggtcgtctcgaccatgtgcaggtaggagttcaccgcgctgacccggtccacctgcagcccggcctgtccgatcagcccgagcaggccgtccttggtgtgcttcttgccgcccacgttgagcagcagcaacaggtccatcgcggtcgcgaacttcatctccggagtgccgtcgaccaggttctcgatgatcaccacgcgggcgccgggccgaccggctgccaccgcgttgcgcagcgcgaggacggtgctctcgtcgtcccactccaggatgttcttgaacaggtagacgtcggcctgcaccgggatctcccggcggcagtcgcccgccaccagctcggcccggtccgccagggctccgcccacgcgcagccgctcgtcggcgcccgccaccaccgcgggcaggtcgaacagcaccccgttcagcttttcgttgcgggacagcagggtggccaggacgttgccctgtccgcccgcgatgtcggccagccgctcggccccggtcaggtcgagccggtcggcgatggccagggcggagagcttgctggactgggtcatggccttgtcgaagacctcggccgactccggccactgctcgtgcaggtactcgaagaagtccttgccgtgcaagtcgtcgaagacgttcttgccggtgcggacggcctggtcgagatggggccacagctcccaggtccacggctcggtgccccacagcaggatgtgcttgaggctgcgtggcgcgtcctcgcgcagcagccgggaggcgtcggtgtgcacgaacttgtccggctccggttcggcgaacacctcgaaacaggtcagcgaccgcatcaggcggcgcagggcgcccgggtccgcgtgcacggccttggccagctcgtccaccgtcgcgggctggtcgtcgagaacgtcggcgaggccgagtttggcggcagcgcggaccgaggcggcgcacgccgcgctgagcgccagctcgcggactcgcatcaccgcggcgggaagaacggtacctgtcatgactgcttacctttctgctgggtgggggctgtggaggtctgtgaaggggcccttcacggacttcgtggcggtcggagctaggcgccgtgttcggcgcgcagcacgaacggagcgggctccgcggtcgcgtgcttcatggcctcgccgaccggctcgcggaaacccttgcccgcggtcgcccgctggtggtcctcggcgctgtcccattcggcgatctcggtgaacaccgcttcatcccgggccgactggtagagccggtgcgaccggaagccgggctgcgcggtcatgtgcgcggtgatctggccgagcagccgccggaactcggtggcgtcgccggtgacggtgaaccggttgatgaaggtgaacacggtcttgctcctgttctgtcgggcgatttccgatgcgccatcggcatttctcgcgtggatctgttcgcgcagcgcacggcgttgcagcttcccgttgggggagcggggaatccggtcggtcagttcgacgtggcgcaggtgcgcgtagtacggcagccgttcggcaacgaactcggcgagctgcgccggattgagcccggcgcccttgggcaccaccagcccgtacgcgaccgatccgctgaaatcgtcggggtagtccagtaccacgcagtcggccacctcggggtgactcagcacgacccgctcgatctccgtcggcgacacgagccagttgtcgcacttgaagacgtccttgatccggtcgaccacgaacagcacgccctcggcgtcgacgtagccgacgtccccggtgctgaaccagccgtcgggatcgacgtcctggcccggctcgcggccgaggtagccgagcatcagctgcgggccacggacctggatctcgcccttttctcccaccggcagtaccgcgcgggagtccacgtcgacgatcctgcagccggtgcccgccaccggccgtcccgacgagcccgttttggggcaggacaaggaatcgaagtgcgtcgacggcgacgtctcctgaagtccgtacccttggacgaccggcaccccgaaacagccggtcagcgcgcgggtcgacggcagcggcagcgccgagccgccgcacaggatgacctgcagtgcgtccgccgccagcgtggacaggcgcgggtgcaccgccagtcgggacagtcgcatgggcaggctgtagaagtgcgtggccttctcattgtcggcggcgtccacggcctgagccacgtcgtcgccgacgtgcagcaccagcgtggcggcgaaggtgaccgccatcgtcaggtgcatcaggtggaaggtcggcagatagttgaacagcaccgacgacggggtgatcccgtgcgcgtgtccggactgggccgcgttgacggtgatgttgcggtggctgagccggaccgccttggctgctcccgtggtgccgctcgtgaactgcaggcaggccacggaattctcgtcgcaagtggtggcgggcagcacgccgtccggtgcggacgcgaggacctcgtccagggtcggcggaccggacggttcggtctggtcgtcgcgatgggtcagcacgaggtgccgcagctcgggcagccggtccagcaccggcaccagcctgcgatgcacctccggcggcacgatcgccgtgaccgcgcccgacgtgcccagcacgtgcaccagtccgtccgccagcagcagcgggttgatcagcgcgggcaccgcccccgacctggcgataccgaagaacgacacggcgaacgccgggtcgagtacccccgccagcgccaccacttgccccggttcggccagctgcgcgtggacggcccgcgccagtctgccggctcgctcatcgagcgccgcgtagctcagttcgcaggctgccgcacgaatcgccacgtgctcgggcgcgcgttcggcggccgaacgcagcaggtcgtcgaccctggggccgccgatgaaccccgcgatcccggtcaacgggtcgccggacaccgtgctgtcctgggcgacggtcgtctcgggcgtagccatgtatccctcctccgctggttgggtttcagttcctcggcaggtcgtcgcggtacacctggcgctcgcgcaccaggagcgcgccgccctcgcgcaccagcacgtccacgcagtcacagctgaggtcgaccgcagccttgccacccagcggcgtaccgatgatcaccgcgtagctcttggccacgatgctgccgtcgggctgcggatcgacctgcaccatgcccagccagtgccgccgctggatgccctgttccgccaggcgggtggccgcttcgagcgcgccggcctcgatggccgtccggccgcgggcgggttcggggtgcgcgttggcggcgaacatcccgtcctcggtgaaggtcttggcccactcctgcacgcggccctcgtccaggtagcgcatctggcgcccgtagaactgctggacctccacgtacagcgcgatgtcctcggtgctgccgactgcctgtaccgcctgtgacatgactgctccctcgcttgctcgtggtcggcaaacgatggcaccggggcatcggacgccactcaactgaggacgaccggcaccggctcgaaccgggcgctgatcggacttgagccgccgggtacagcctggtcccgttcacaacggacgggctggagggaaggctgatgaggagcaccgaggagccgcgggtcgccttggtcacgggagcgaccagcgggatcggtctcgccgtcaccaaggcgctggccgccaggggactgcgggtgttcatctgcgcgcgcaaccgggagaacgtcgtttccacggtgaaggagttgcgggcgcagggtttggacgtggacggccaggccggtgacgtgcggtcggtggccgccgttcgcgaggtggtcgagtccgcggtcaaccggttcggcaccatcagcgtgctggtcaacaacgccggccgcagcgggggcgggatcaccgcgaagatcaccgacgagctgtggcaggacgtgatcgacaccaatctcaacagcgtcttcacggtcacccgcgaggtgctgaccaccggcgggctcgacggggccgacggcggccggatcatcaacatcgcctcgaccgggggcaagcagggcgtcccgctcggcgcgccctactcggcggccaagagcggggtcatcggcttcaccaaggcgctgggcaaggaactggccaagaccggggtcacggtgaacgccgtctgccccggctacgtcgaaacgccgatggccgtccgcgtccggcaggcgtatgccagcacttgggacaccaccgacgaaaacgtgctggcgcggttcaacgacaagatcccgctcggccgctactgcactccggaagaggtggcgggcatggtcgactacctggtcgccgacacggccgcttcggtgacggcgcaggcgatcaacgtctgcggcggcctcggcaactactgaccccgcacgctcacggaaaggcacagccatgcccgccgcagcacagcagcacaccgaacaccggatcgacatcgacgctcccgccggcctggtgtaccgcatcatcgcggacgcgaccgagtggccgcggcacttcaccccgaccgtccacgttgatcagtccgaactggacggacacaccgaacgcctgcacatctgggccaacgccaacgggcagctgaagagctggacctcgctccgggaactcgacgagcgggccggccggatccggttccggcaggaggtgtccgcgcccccggtcgcctcgatgagcggcgagtggatcgtgtccgagcgggtcgcggagcgcaccacgctggtgctgacgcacgacttcgccgcggtggacgacgatcccgccggagtggaatggatcaccaaggccaccaacgggaacagtgacaccgaactggcgaacatcaaggcactggccgagcgctgggagcggatggaccggctggccttcgacttcgaggactccgtgctggtacgggcgcccaaggaacgggcgtaccacttcctggaccgcgtggacctgtggccggaccggctgccccacgtcgccaggctggagctgcgcgaggacgtgcccggagttcagcacatgtccatggacaccaaggcgaaggacggatcgacgcacaccacggtgtcggtgcgggtgtgcttccccgaagcccggatcgtctacaagcaactcgtgccgcccgcgttgctgaccacccacaccggagtgtggacgttcgaggacacagcggacggtgttttggtgacttccgcgcacacggtcgtgctcaacgaggccaacatcggcacggtgcccggcccggccgcgactgtcgagtcgacgcgcgacttcgtccgcaacgcgatcagcggcaacagccaggccacgctgcgtcatgccaaggcgttcgccgaggcgaccgatgcctgaggactccggcgaagagcccgaagtcctcgtggccggggcggggccggtcgggctgaccgccgcgcacgagctggcccgccgaggggtgcgggtacggctggtggaccgtagcgccggcccggccacgacgagtcgtgcgctcgcgacgcacgcccgcacgttggagatctggcaccagatggggctgctcggcgagctgctgccgcggggccggcgggtcgagcacttcacactgcatctgaagggtaaaaccctgatgtgcttcgacaccaactatgacaccatgccgacccggttcccgttcagcctcatggtggaccaggtcgtgaccgaggaagtgctgcgccggcaggtgcgggcactcggggtcacggtcgagtggggcgtcgagctgacctggttcgaccaggagccggacggggtcctggcggagctgcggcatgcggacgggacagtcgagcaggtcaccgcggcctggctggtcggcgcggacggtgcccgcagcaccgtccgcaagcggttggacctgcgcctgcagggggactccacacagacctggctcaacgccgacgtggtgctcgacacggatctggccggcgacagcaaccacctgctgcacaccggacgcggcaccttgctgctggttccctttccagaaccgggaaagtggcgggtggtcgacaccgaggacaccgaccacgccgacgatgcccggatcgtccgggccaggctggccgacaagctcaccagggcgctcggccgccccatcgaggtgcccgagccgagctggatctcggttttcaccgtgcagcagcggatgatcgaccggatgcgggccggccgttgtttcgtcgccggtgacgccgcccatgtgcacagcccggcgtccgggcagggcatgaacaccgggatccaggacgcctacaacctggcgtggaagctggcggacgtggtgcgcggccacgccaaggagagcctgctcgacagctatggggccgagcgggtgccgatcggcgagacgttgttgcgtaccaccaggaccgccactgcgcttgtgagcctgcgcaacaccgtcgcgccgttggtcatgccggccggaacccggctgctgggggcgctcaagccactcaaacggcggatcgagcgcaccatgatccggggcttctgcgggctgaccctgaactacacccacagcccgctgagcctggcctgcgcaaccccggacggcatccagccgggacatcgggtgggctgctcggtggatcgtgcccggacctcaccgggatggcaagggctggtcacggaactgaccgatccccggtggactttgctcgccttcgccgattcccaggaacagcggcagatcgccgcgcaagtcgagcggcgctacggcaaggccgtctcggtgcgggtggtggccgaggccgccacgagcgagcgggttcttgccgacccgggcgatgacctagcacgagatttcgccatgcgggcagggtatttcgtgctgatccggccggacggccacctggcggcgaaagggcggctgagcgatgacctggatggcgcattcggcgctctgggactcgtgccggccgatgccgggggagacccggcccaccacctcgattccgaaggatcacgcatgcgcatcatcgacctgtccgcgaccatggacgctgccgatcgctgggaggccaacccggtcacccacgaggtgctcacggccgccgaaggcgcgcagcacatggcggcggagatgaaagagcacttcgggatcgacttcgacccctcggtgctgcccggcggggagttgctcacgctggacacgttgacgctgaccacgcacaccggcacgcacgtcgacgccccgtcgcactacgggactccccgcgacggcgtggccaggcacatcgaccagatgccgctggagtggttcctgcggccgggggtggtcctcgacctcacgggcgagccggtcggcgcggccggtgcggaccggctgcgcgaggagttcgagcgaatcggctataccccgaagccgctggacatcgtgctgctcaacaccggcgccgacgcgctggccggaagcccgaagtacttcaccgacttcaccgggctcgacgggaaggccaccgagctgctgctcgatttgggggtacgggtcatcggtaccgacgcgttcagcctggacgcgccgttcggtcacatgatcgccgaataccggcgtacgggggatcgctccgtgctgtggcccgcgcatttcgcgggccgggaccgggagtactgtcagatcgaggggttgaccaacctggccgcgctgccgtcgccgaccggtttttcggtatcctgcctgccggtcaagatcgccggcgcgggcgccggctggacccgggccgttgccctgctggactaggaaggaaggtccgtgaaggggcccttcacggactcagagtccgtgaagggccccttcacagccgcggtgaccgatgtatcgggagatacatgccggcgtcgggcgatgcccttccttggatgtactcctcttgagcggccctcggctgggggagtaccaataccgtatgaaattcaacctgcttggtccgatggaagttctgtgcgccgatggcacggtgacgcccagcgccgcgaaaatgcgctggattctggcattgctcctgttgcacggaaatcgagtggtcgatcaggcttcgatgatcgacgaattgtggggagatcacccgccgcgcagtgcggtgaccaccacgcagacctacgtttaccaattgcgcaagaagtacgactactacgcacaacgagaggggagaaagagtttcatcgtcacccgggcgcccggttatctgctgcagctggacgacgatcagctcgatgtccgaagattccagagactcagcgccgagggcagcgccctgttctcggcgggccacgccgaacgggcggacgaggtgctgcggcaggcactgcggctgtggcggggccccgcgctggccggcatcgcacccggccgcatgctgcaggcgcacgtcgcctacctggaagaggcccggctgcgcaccgtgcaggtgcgcatcctcgccgacgcggcactcgggcggcaccgggatctgattccggagctgcggtcactggtgatcgagcatccgctcgacgaatggtttcaccagcagctgatcacggcactggccgaggcagggcgcaggggagacgccctgcacgcctgccgggtcctgcaccgcaccctcgccgacgaactcggcgtggcgccctccgagccgctgcgcaagctccagcaggacctgctgaccggccacgtccggcgggcacccgcgcatgtgtgacctgcggtggagccggagacggcggccgccgccgtctccggcctcacgcggggaccagcggcgccgcggtctgcgccttgttcaccgcgtccagccgggaaaccgcgttcagcttcacgaagatgttgcgcaggtggcgtttcaccgtcccctgggtgatggccaggcaattcccgatctgtgcgttggtcatcccgcgggcgaccagcgacatgatctcgtgctcgcgcctggacagcggagtacgcaggttcgaggacagcgaccggacgtcgacactcggcaccgagaggaagacgcagcgctcgtcgcggctcacctcgcgcaccaccgagagcaggtacatgtcggagacattcttgggcaggtaaccccggacgccgatgcccaggaattcctgaatgaaaccgaatctgttctcctggctcaggatgacgacgggcttccccggagccgccgccgacaattcgagcaggccggattcgacttccggaccccagccgtcgactccatagagaatgacgtccacaattcccctggagacactgtcgagtgcttcgccgatggtcgccaccgcgtcaatggcggccagatcgtcggcgccttgaatcagcgaaagcagcccgacgcgaaaaacggaactctcttcgacgatcagaattcttacacggtcagttatcatggtgtgataacactcccccggtatggtcaaccgtgccagcccgcgcggctggcggttccttcgtatagtcccccgactgcgtggcataaaaattggcgga

SEQ ID NO: 27 - protein, 20 aa, Amycolatopsis sulphurea
GPVGLVSTGCTSGVDVIGHA

SEQ ID NO: 28 - protein, 24 aa, Amycolatopsis sulphurea
VPVSSIKSMVGHSLGAIGSLEVAA

SEQ ID NO: 29 - protein, 9 aa, Amycolatopsis sulphurea
VSEQAGGLD

SEQ ID NO: 30 - protein, 6 aa, Amycolatopsis sulphurea
LGYDSL

SEQ ID NO: 31 - protein, 17 aa, Artificial Sequence
  Conserved sequence to be found in ChdOI and ChdOII
  SITE (2)..(2): Xaa can be any naturally occurring amino acid
  SITE (4)..(5): Xaa can be any naturally occurring amino acid
  SITE (7)..(9): Xaa can be any naturally occurring amino acid
  SITE (11)..(16): Xaa can be any naturally occurring amino acid
GXGXXGXXXAXXXXXXG

SEQ ID NO: 32 - protein, 11 aa, Artificial Sequence
  Conserved motif to be found in ChdY
  SITE (2)..(2): Xaa can be any naturally occurring amino acid
  SITE (6)..(6): Xaa can be any naturally occurring amino acid
  SITE (8)..(8): Xaa can be any naturally occurring amino acid
  SITE (10)..(10): Xaa can be any naturally occurring amino acid
HXGTHXDXPXH

SEQ ID NO: 33 - DNA, 22 bp, Artificial Sequence
  Primer
tcctcactgcaggtcgagtacc

SEQ ID NO: 34 - DNA, 18 bp, Artificial Sequence
  Primer
cgggaagtcgcggtatgc

SEQ ID NO: 35 - DNA, 20 bp, Artificial Sequence
  Primer
cgcgctggtcaaagtctacg

SEQ ID NO: 36 - DNA, 18 bp, Artificial Sequence
  Primer
ctggacgcctcgccgtac

SEQ ID NO: 37 - DNA, 30 bp, Artificial Sequence
  Primer
tatatacatatgaaggacaatctcgcgaga

SEQ ID NO: 38 - DNA, 29 bp, Artificial Sequence
  Primer
tatatatctagaggacctccgcatcaggc

SEQ ID NO: 39 - DNA, 40 bp, Artificial Sequence
  Primer
agtcgaattcgcaccatatgagaccaagcgcgtccgggtg

SEQ ID NO: 40 - DNA, 43 bp, Artificial Sequence
  Primer
cgactctagaggatcactagttaccagcccgacccgagcacgc

SEQ ID NO: 41 - DNA, 24 bp, Artificial Sequence
  Primer
tatatagcatgcgcatcatcgacc

SEQ ID NO: 42 - DNA, 19 bp, Artificial Sequence
  Primer
gcgtcggtgctgatgaccc

SEQ ID NO: 43 - DNA, 19 bp, Artificial Sequence
  Primer
ggtcatcagcaccgacgcg

SEQ ID NO: 44 - DNA, 33 bp, Artificial Sequence
  Primer
tatataactagtcgtccagctgcagcagataac

SEQ ID NO: 45 - DNA, 28 bp, Artificial Sequence
  Primer
atatacatatgcgcatcatcgacctgtc

SEQ ID NO: 46 - DNA, 32 bp, Artificial Sequence
  Primer
tatatatctagactagtccagcagggcaacgg

SEQ ID NO: 47 - DNA, 26 bp, Artificial Sequence
  Primer
atatacatatgcctgaggactccggc

SEQ ID NO: 48 - DNA, 31 bp, Artificial Sequence
  Primer
tatatacatatgcgcatcatcgatctgtcga

SEQ ID NO: 49 - DNA, 30 bp, Artificial Sequence
  Primer
atatatctagactactcctccaccaccgcc